(19) United States
(12) Patent Application Publication
(10) Pub. No.: US 2003/0147770 A1
(43) Pub. Date: Aug. 7, 2003

(54) Light treatment monitoring and data collection in a fluid treatment system using light for the treatment of fluid products

(75) Inventors: Eddie Lee Brown, San Diego, CA (US); Edward Domanico, San Diego, CA (US); William M. Fries, San Diego, CA (US); Jeffrey M. Holloway, San Diego, CA (US); Richard E. May, San Diego, CA (US); Kenton J. Salisbury, San Diego, CA (US); John S. Thompson, San Clemente, CA (US)

Correspondence Address: FITCH EVEN TABIN AND FLANNERY, 120 SOUTH LA SALLE STREET, SUITE 1600, CHICAGO, IL, 60603-3406, US

(73) Assignee: PurePulse Technologies, Inc., San Diego, CA (US)

(21) Appl. No.: 10/150,681

(22) Filed: May 17, 2002

Related U.S. Application Data
(60) Provisional application No. 60/291,850, filed on May 17, 2001.

(51) Int. Cl. B08B 17/00; A61L 2/00; G01D 11/26; B01J 19/08; A61L 9/00

(52) U.S. Cl. 422/24; 422/3; 422/6; 422/38; 422/119; 422/186; 422/305

(57) ABSTRACT

Methods and apparatus for precisely monitoring and collecting data relating to the light treatment of a product in a treatment system. In one implementation, a method for use with a treatment system using light comprises the steps of: illuminating a product with a light treatment comprising light having a spectrum of wavelengths within a range of 170 to 2600 nm, the light treatment for treating the product; and measuring a fluence of a portion of the light treatment for each of a plurality of wavelengths of the spectrum of wavelengths simultaneously. In preferred implementations, the light treatment is a pulsed light treatment and the product is a biological fluid product flowed through a treatment chamber. Furthermore, in preferred implementations, the light treatment is for the deactivation of microorganisms, such as viruses, bacteria, fungus, and other pathogenic and non-pathogenic microorganisms.

This application is a Non-Provisional application of and claims priority under 35 U.S.C. 119(e) to U.S. Provisional Application No. 60/291,850, of Fries et al., entitled SYSTEM FOR DECONTAMINATION OF FLUID PRODUCTS USING BROAD SPECTRUM LIGHT, filed May 17, 2001, which is incorporated herein in its entirety by reference.

This patent document relates to the following patent documents, which are incorporated herein in its entirety by reference: U.S. patent application Ser. No. 09/976,597, of Fries et al., entitled SYSTEM FOR THE DECONTAMINATION OF FLUID PRODUCTS USING LIGHT, filed Oct. 12, 2001, Docket No. 70683; and U.S. patent application Ser. No. 09/976,776, of Fries et al., entitled FLUID FLOW PATH FOR A FLUID TREATMENT SYSTEM USING LIGHT FOR THE DECONTAMINATION OF FLUID PRODUCTS, filed, Oct. 12, 2002, Docket No. 71715.

This patent document relates to the following patent document filed concurrently herewith, which is incorporated herein in its entirety by reference: U.S. patent application No. ______, of Brown et al., entitled LIGHT TREATMENT CONTROL IN A FLUID TREATMENT SYSTEM USING LIGHT FOR THE TREATMENT OF FLUID PRODUCTS, Docket No. 72722.

### BACKGROUND OF THE INVENTION

1. Field of the Invention

The present invention relates generally to treatment systems using a light treatment for treating products. Even more specifically, the present invention relates to the monitoring of the light treatment and collection of data related to the light treatment in such treatment systems.

2. Discussion of the Related Art

Treatment systems using a light treatment for the deactivation of pathogens such as viruses, bacteria, fungus, microorganisms or other harmful substances on or within a target product have been in use for several years. For example, many treatment systems exist that expose a product to continuous wave ultraviolet (UV) light radiation produced by Mercury lamps. Also, treatment systems exist that expose a product to pulsed light energy having a broad spectrum, such as produced by Xenon flashlamps.

In several treatment systems, it is desired to quantify or measure the light treatment illuminating a given product to be treated to determine its effectiveness or to assess whether the light treatment provides the proper illumination to the product. In some systems, photo-sensitive detectors have been employed to measure a fluence level or intensity of the light treatment. For example, as described in U.S. Pat. No. 5,925,885, to Clark, et al., entitled PARAMETRIC CONTROL IN PULSED LIGHT STERILIZATION OF PACKAGES AND THEIR CONTENTS, issued Jul. 20, 1999, which is incorporated herein by reference, a photodetector is positioned to receive light emitted from a pulsed light source and to measure the fluence-per-flash or energy within a given bandwidth of the light treatment. Such detectors output a single measurement representing the energy of the received light across the spectrum of wavelengths received at the detector. Often a spectral filter may be used to filter the light received at the photodetector. These measurements are typically used for the parametric control of the light treatment.

Recently, sensitive biological and pharmaceutical fluid products, such as blood products, are being treated in such treatment systems. It has been found that special care must be taken when treating such sensitive biological fluids to avoid damaging the properties of the fluid (e.g., reducing the protein activity of the blood product) while at same time deactivating pathogens or other contaminants to the desired level. It is often important not to damage these types of products since they may be unusable if damaged too much. Additionally, certain biological fluids can be very expensive and not easily replaceable.

Thus, a need arises for more precise methods of monitoring the light treatment and collecting data related to the light treatment such that the provided treatment process can be precisely controlled to ensure the proper treatment with minimal damage to the product.

### SUMMARY OF THE INVENTION

The present invention advantageously addresses the needs above as well as other needs by providing various methods and apparatus for precisely monitoring and collecting data relating to the light treatment of a product in a treatment system.

In one embodiment, the invention can be characterized as a method for use with a treatment system using light comprising the steps: illuminating a product with a light treatment comprising light having a spectrum of wavelengths within a range of 170 to 2600 nm, the light treatment for treating the product; and measuring a fluence of a portion of the light treatment for each of a plurality of wavelengths of the spectrum of wavelengths simultaneously.

In another embodiment, the invention can be characterized as a treatment system using light comprising: a light source for providing a light treatment, the light treatment having a spectrum of wavelengths within a range of 170 to 2600 nm; a treatment chamber containing a product to be treated with the light treatment, the light treatment for treating the product; and a spectrometer having an input collector positioned to receive a portion of the light treatment, the spectrometer for measuring a fluence of the portion of the light treatment for each of a plurality of wavelengths of the spectrum of wavelengths simultaneously.

In another embodiment, the invention may be characterized as a method for use with a system for the deactivation of microorganisms using light comprising the steps: illuminating a product with a light treatment having a spectrum of wavelengths, the product being transmissive to at least 1% of light having at least one wavelength within a range of 170 to 2600 nm, the light treatment intended to treat the product; measuring a fluence level for a portion of the light treatment illuminating the product for each of a plurality of wavelengths of the spectrum of wavelengths; measuring a fluence level for a portion of the light treatment transmitting through the product for each of the plurality of wavelengths of the spectrum of wavelengths; and generating an absorption profile across each of the plurality of wavelengths for the product based upon a comparison of the results of the measuring steps.

In another embodiment, the invention can be characterized as a monitoring system for use with a treatment system for treating products using light comprising: a light source for illuminating a product with a light treatment having a spectrum of wavelengths, the product being transmissive to at least 1% of light having at least one wavelength within a range of 170 to 2600 nm, the light treatment intended to treat the product; a first optical detector positioned to measure a fluence level for a portion of the light treatment illuminating the product for each of a plurality of wavelengths of the spectrum of wavelengths; a second optical detector positioned to measure a fluence level for a portion of the light treatment transmitting through the product for each of the plurality of wavelengths of the spectrum of wavelengths; and a controller coupled to the first optical detector and the second optical detector for generating an absorption profile across the plurality of wavelengths for the product based upon a comparison of the results of the measuring steps.

In another embodiment, the invention may be characterized as a method for use with a treatment system using light comprising the steps: illuminating a treatment chamber with a light treatment having a spectrum of wavelengths, the treatment chamber transmissive to at least 1% of light having at least one wavelength within a range of 170 to 2600 nm, the treatment chamber being empty but adapted to flow a product therethrough that is to be treated with the light treatment; measuring a fluence level for a portion of the light treatment illuminating the treatment chamber for each of a plurality of wavelengths of the spectrum of wavelengths; measuring a fluence level for a portion of the light treatment transmitting through the treatment chamber for each of the plurality of wavelengths of the spectrum of wavelengths; comparing the respective fluence levels measured for each of the plurality of wavelengths; and determining, based upon the comparing step, whether the treatment chamber is ready for the product to be flowed through the treatment chamber for operation.

In another embodiment, the invention can be characterized as a monitoring system for use with a treatment system using light comprising: a light source for illuminating a treatment chamber with a light treatment having a spectrum of wavelengths, the light treatment having a known fluence level at each of a plurality of wavelengths of the spectrum of wavelengths; the treatment chamber transmissive to at least 1% of light having at least one wavelength within a range of 170 to 2600 nm, the treatment chamber being empty but adapted to flow a product therethrough that is to be treated with the light treatment; a first optical detector for measuring a fluence level for a portion of the light treatment illuminating the treatment chamber for each of the plurality of wavelengths of the spectrum of wavelengths; a second optical detector for measuring a fluence level for a portion of the light treatment transmitting through the treatment chamber for each of the plurality of wavelengths of the spectrum of wavelengths; and a controller coupled to the first optical detector and the second optical detector, the controller adapted to perform the following steps: comparing the respective fluence levels measured for each of the plurality of wavelengths; and determining, based upon the comparing step, whether the treatment chamber is ready for the product to be flowed through the treatment chamber for operation.

In another embodiment, the invention may be characterized as a method for use with a treatment system using light comprising the steps: flowing a buffer fluid through a fluid flow path of the treatment system, the buffer fluid having known physical and optical absorption properties across a plurality of wavelengths of a spectrum of wavelengths; illuminating the buffer fluid with a light treatment having a known fluence level at each of the plurality of wavelengths of the spectrum of wavelengths, a portion of the fluid flow path and the product are transmissive to at least 1% of light having at least one wavelength within a range of 170 to 2600 nm; measuring a fluence level at one or more of the plurality of wavelengths for a portion of the light treatment transmitting through the buffer fluid; verifying, based on the measuring step, the optical absorption properties of the buffer fluid; determining, based upon the verifying step, whether the optical properties of the fluid flow path are within an acceptable range for operation.

In another embodiment, the invention can be characterized as a monitoring system for use with a treatment system using light comprising: a fluid flow path of the treatment system for flowing a buffer fluid therethrough, the buffer fluid having known physical and optical absorption properties across a plurality of wavelengths of a spectrum of wavelengths; a light source for illuminating the buffer fluid with a light treatment having a known fluence level at each of the plurality of wavelengths of the spectrum of wavelengths, wherein a portion of the fluid flow path and the product are transmissive to at least 1% of light having at least one wavelength within a range of 170 to 2600 nm; an optical detector positioned to measure a fluence level at one or more of the plurality of wavelengths for a portion of the light treatment transmitting through the buffer fluid; and a controller coupled to the optical detector, the controller adapted to perform the following steps: verifying, based on the measuring step, the optical absorption properties of the buffer fluid; and determining, based upon the verifying step, whether the optical properties of the fluid flow path are within an acceptable range for operation.

In another embodiment, the invention may be characterized as a method for use with a treatment system using light comprising the steps: flowing a buffer fluid through a fluid flow path of the treatment system, the buffer fluid having known physical and optical absorption properties, the flowing establishing an operational condition of the treatment system; determining whether the operational condition has been established; flowing a fluid product through the fluid flow path, the fluid product to be treated with a light treatment; and illuminating the fluid product with the light treatment.

In another embodiment, the invention can be characterized as a treatment system using light comprising: a fluid flow path of the treatment system for flowing a buffer fluid therethrough to establish an operational condition of the treatment system, the buffer fluid having known physical and optical absorption properties; means for determining whether the operational condition has been established; means for flowing a fluid product through the fluid flow path, the fluid product to be treated with the light treatment; and a light source for illuminating the fluid product with a light treatment.

In another embodiment, the invention may be characterized as a method for use with a fluid treatment system using light comprising the steps: illuminating a treatment chamber of a treatment system with a light treatment, the treatment chamber containing a product to be treated with the light treatment, a portion of the treatment chamber and the product transmissive to at least 1% of light having at least one wavelength within a range of 170 to 2600 nm; measuring a fluence level of a portion of the light treatment transmitting through the treatment chamber at a first location proximate to a first portion of the treatment chamber; and measuring a fluence level of a portion of the light treatment transmitting through the treatment chamber at a second location proximate to a second portion of the treatment chamber, the second location positionally offset from the first location, the first location and the second location within a portion of a profile of the treatment chamber.

In another embodiment, the invention can be characterized as a light treatment monitoring system comprising: a treatment chamber for containing a product to be treated with a light treatment, at least a portion of the treatment chamber and the product transmissive to at least 1% of light having at least one wavelength within a range of 170 to 2600 nm; a first optical detector positioned to measure a fluence level of light transmitting through a first portion of the treatment chamber; and a second optical detector positioned to measure a fluence level of light transmitting through a second portion of the treatment chamber, the second portion positionally offset from the first location.

In another embodiment, the invention may be characterized as a light treatment monitoring system comprising: a treatment chamber for containing a product to be treated with a light treatment, a portion of the treatment chamber and the product transmissive to at least 1% of light having at least one wavelength within a range of 170 to 2600 nm; an optical detector positioned to measure a fluence level of light transmitting through a first portion of the treatment chamber; and a position adjustment structure coupled to the optical detector, the position adjustment structure moveable in one or more directions to reposition the optical detector at different locations within a portion of a profile of treatment chamber.

In another embodiment, the invention can be characterized as a method of fluid decontamination comprising the steps: flowing a fluid product through a treatment chamber, the fluid product and the treatment chamber transmissive to at least 1% of light having at least one wavelength within a range of 170 to 2600 nm; illuminating the fluid product and the treatment chamber with at least one pulse of light; measuring an amount of the light illuminating the fluid product and the treatment chamber; and measuring an amount of the light transmitting through the fluid product and the treatment chamber.

In another embodiment, the invention may be characterized as a monitoring system for a fluid treatment system comprising: a light source for providing pulses of light; a treatment chamber positioned to receive the pulses of light, wherein a fluid product to be treated flows therethrough, wherein at least a portion of the treatment chamber and the fluid product are transmissive to at least 1% of light having at least one wavelength within a range of 170 to 2600 nm; a first process monitor for measuring a fluence level of the pulses of light provided by the light source that illuminate the treatment chamber and the fluid product; and a second process monitor for measuring a fluence level of portions of the pulses of light transmitting through the treatment chamber and through the fluid product.

In another embodiment, the invention may be characterized as a method of calibrating a spectroradiometer comprising the steps: calibrating a first spectrum of wavelengths of an operating spectrum of the spectroradiometer with a first calibration light source, the first calibration light source not providing an accurate calibration of the spectroradiometer in the first spectrum of wavelengths; calibrating a second spectrum of wavelengths of the operating spectrum of the spectroradiometer with a second calibration light source, the second calibration light source providing an accurate calibration of the spectroradiometer in the second spectrum of wavelengths, a portion of the first spectrum of wavelengths overlapping the second spectrum of wavelengths; and adjusting the calibration of the first spectrum of wavelengths based on a difference between the first calibration and the second calibration at the portion of first spectrum of wavelengths overlapping the second spectrum of wavelengths to generate an absolute irradiance calibration file that is sufficient to calibrate the spectroradiometer across the first spectrum of wavelength and the second spectrum of wavelengths.

In another embodiment, the invention can be characterized as a method for use with a spectrometer in a treatment system using light comprising the steps: generating a transmission file corresponding to a filter used to attenuate light input to the spectrometer, the filter non-uniformly transmitting light within a transmission spectrum through the filter, the transmission file generated on a per wavelength basis; and compensating the calibration of the spectrometer based on the transmission file, such that readings of the spectrometer account for non-uniform transmission of the filter on a per wavelength basis.

### BRIEF DESCRIPTION OF THE DRAWINGS

The above and other aspects, features and advantages of the present invention will be more apparent from the following more particular description thereof, presented in conjunction with the following drawings wherein:

FIGS. 1, 2 and 3 are a front perspective view, a rear perspective view and a front view, respectively, of a fluid treatment system using a light source emitting e.g., pulsed polychromatic light, such as broad spectrum pulsed light (BSPL), according to one embodiment of the invention;

FIG. 4 is an external view of the fluid treatment system of FIGS. 1-3;

FIG. 5 is a perspective view of a syringe mount assembly of the fluid treatment system of FIGS. 1-3 according to one embodiment of the invention;

FIG. 6 is a schematic view of the fluid flow path components of the fluid treatment system of FIGS. 1-3 according to another embodiment of the invention;

FIGS. 7A and 7B, are a perspective view and a side view, respectively, of one embodiment of a treatment chamber of the fluid flow path of FIG. 6;

FIG. 7C is a schematic view of a transition from a circular flow profile to a substantially flat profile at the input and output of the treatment chamber of FIG. 7A and 7B according to another embodiment of the invention;

FIG. 8 is an exploded view of one embodiment of a cartridge as shown in FIGS. 1-3 illustrating the treatment chamber of FIG. 7 positioned therein;

FIGS. 9A and 9B are cross sectional views of the cartridge of FIG. 8 containing the treatment chamber of FIGS. 7A-7B according to one embodiment of the invention;

FIG. 10 is a perspective view of the cartridge of FIG. 8 as positioned within a cartridge registration plate of the fluid treatment system of FIGS. 1-3 in accordance with one embodiment of the invention;

FIG. 11 is a perspective view of another embodiment of the fluid treatment system of FIGS. 1-3;

FIG. 12 is a perspective view of a flat, disposable treatment chamber that may be used in the fluid treatment system of FIGS. 1-3 in accordance with another embodiment of the invention;

FIG. 13 is a perspective view of a reusable, non-disposable treatment chamber according to another embodiment of the invention;

FIG. 14 is a perspective view of a treatment chamber that may be used in the fluid treatment system of FIGS. 1-3 in accordance with another embodiment of the invention;

FIGS. 15A and 15B are a simplified front view and side view, respectively, illustrating the relationship between the treatment chamber, the light source and the respective process monitors according to one embodiment of the invention;

FIG. 16A is a simplified side view of a variation of the process monitoring system of FIG. 15B according to another embodiment of the invention;

FIG. 16B is a diagram illustrating one embodiment of the spectroradiometer of FIG. 16A to allow for the simultaneous measurement of discrete fluences at multiple wavelengths of the spectrum of a light treatment;

FIG. 16C is an illustration of a treatment system using multiple spectrometer devices for measuring incident and transmitted light according to one embodiment of the invention;

FIG. 16D is a flowchart of the steps performed in accordance with one embodiment of the invention;

FIG. 17 illustrates an absorption profile of a fluid product in accordance with one embodiment of the invention;

FIGS. 18, 19, 20 and 21 are flowcharts illustrating the steps performed in various embodiments of the invention;

FIG. 22 is a flowchart illustrating the steps performed according to one embodiment of the invention in which fluence measurements are taken of light transmitting through a treatment chamber at multiple locations across the profile of the treatment chamber;

FIG. 23 is a simplified perspective view of a detector system that measures incident and transmitted light at different portions, e.g., entrance and exit portions, of a treatment zone of a treatment system according to one embodiment of the invention;

FIG. 24 is a simplified perspective view of a detector array that is used to obtain the spectral profile of the light treatment across the entire treatment chamber according to yet another embodiment of the invention;

FIG. 25 is a simplified perspective view of process monitors integrated on an adjustable x-y translation table used to obtain the spectral profile of the light treatment across different portions of the treatment chamber according to yet another embodiment of the invention;

FIG. 26A is a diagram illustrating a light source used for the calibration of a spectrometer in accordance with one embodiment of the invention;

FIG. 26B is a fluence vs. wavelength plot measured in the calibration of the spectroradiometer according to one embodiment of the invention;

FIG. 26C is a flowchart illustrating the steps performed in the calibration of a spectroradiometer, such as illustrated in FIG. 26A, in accordance with one embodiment of the invention;

FIG. 27A is a diagram illustrating one method of attenuating light received at a spectrometer for use in a treatment system using light for the treatment of products according to one embodiment of the invention;

FIG. 27B is a flowchart illustrating the steps performed to calibrate a filter to be used the attenuation of light input to a spectrometer while maintaining the spectrometer calibration;

FIG. 28 is a simplified side view of a treatment chamber including a spectral filter positioned between the treatment chamber and the light source according to another embodiment of the invention;

FIG. 29 is a simplified side view of a treatment chamber including a device to cool the treatment chamber due to the heat energy of the light illuminating the treatment chamber according to another embodiment of the invention;

FIG. 30 is a flowchart illustrating the steps performed according to another embodiment of the invention in which a dimensional boundary of a treatment zone of a treatment system using a light treatment is automatically adjusted;

FIG. 31 is a simplified view of an adjustable fluence light treatment system according to one embodiment of the invention;

FIG. 32 is a flowchart illustrating the steps performed according to another embodiment of the invention in which the fluence level of a light treatment for treating a product is adjustable by an automatic adjustment of the linear distance of the light source to a treatment chamber;

FIG. 33 is a flowchart illustrating the steps performed according to another embodiment of the invention in which the fluence level of a light treatment for treating a product is adjustable according to property changes in the product being treated;

FIG. 34 is a flowchart illustrating the steps performed according to another embodiment of the invention in which the concentration of a fluid product within a buffer fluid to be treated with a light treatment is automatically adjustable;

FIGS. 35A and 35C are simplified views of a system for the measurement and verification of fluence at a given location without using an optical detector at that location and for determining the positioning of a light source according to one embodiment of the invention;

FIG. 35B is a graph illustrating fluence vs distance curves generated for optical collectors at various reference points in FIGS. 35A and 35B;

FIG. 36 is a flowchart illustrating the steps performed according to another embodiment of the invention;

FIG. 37 is a flowchart illustrating the steps performed according to another embodiment of the invention for determining the starting position of a light source relative to a product to be treated;

FIG. 38 is a simple diagram illustrating various particle velocities across a thickness of a fluid flow path of a treatment chamber according to one embodiment of the invention;

FIG. 39A is a flowchart illustrating the steps performed in another embodiment of the invention which is used to set the flash rate of a pulsed light source treatment system;

FIG. 39B is a table of one embodiment of a finite element analysis of run conditions and resulting ratio of centerline to average velocities to be used with design of experiment software for setting the flash rate of a pulsed light treatment;

FIG. 40 is a simplified schematic drawing of a production fluid treatment system scaled to continuously treat fluids according to one embodiment of the invention;

FIG. 41 is a system level diagram of a fluid treatment system according to one embodiment of the invention;

FIG. 42 is a diagram illustrating hardware components of a computer-based control system for a treatment system using a light treatment for treating products in accordance with one embodiment of the invention;

FIG. 43 is a flowchart of the steps performed by the control software in accordance with one embodiment of the invention;

FIG. 44 is a functional block diagram of one embodiment of control software for a computer-based control system for a treatment system using a light treatment;

FIG. 45 is a graph plotting the percentage of protein activity remaining vs. the number of flashes used in EXAMPLE 1;

FIG. 46 is a graph plotting the log reduction of E. coli within a test fluid vs the number of flashes at a high and at a low fluence level according to EXAMPLE 2;

FIG. 47 is a graph plotting the log reduction of E. coli within a test fluid vs time in an extended run test according to EXAMPLE 3;

FIG. 48 is a graph plotting the radiant energy across a wavelength spectrum of light treatment transmitting through a treatment chamber according to EXAMPLE 7; and

FIGS. 49 and 50 are graphs plotting the percentage of protein recovery or protein activity vs. the total energy of BSPL for various fluence levels/flash for Beta-galactosidase in water and BSA, respectively.

Corresponding reference characters indicate corresponding components throughout the several views of the drawings.

### DETAILED DESCRIPTION OF THE INVENTION

The following description is not to be taken in a limiting sense, but is made merely for the purpose of describing the general principles of the invention. The scope of the invention should be determined with reference to the claims.

Described herein are various methods and apparatus involving the use of a light treatment to treat a product. As described variously throughout, such light treatment may be produced by a variety of light sources depending on the embodiment. Thus, as used throughout, the term “light treatment” refers to any type of light treatment, such as continuous wave light treatment or pulsed light treatment. Furthermore, the light treatment may include light having one or more wavelengths. Depending on the embodiment, the “product” to be treated may be solid or fluid (e.g., liquid or gas) and may further be opaque to the light treatment or transmissive to at least a portion of the light treatment. Fluid products may be flowed through a chamber or static within a chamber. For example, in some embodiments, the product is a biological fluid, such as a blood product. Thus, the term product as used herein is meant to include, for example, biological fluids and their derivatives, such as, blood, blood plasma, blood plasma derivatives, bioprocessing fluids and other fluid product, such as drugs and pharmaceuticals, especially bio-pharmaceuticals such as monoclonal antibodies, solutions such as a buffer, glucose and other sugar solutions, culture medias, as well as molecular biology and biochemistry reagents. Such products may be naturally occurring or synthetically produced.

Additionally, depending on the embodiment, the light treatment is used generally for the purpose of “treating” the product. For example, the light treatment is for the purpose of modifying or altering the product, or otherwise stimulating a change in the product. By way of further example, the light treatment is for the alteration, deactivation, or activation of portions of the product. For example, in several embodiments, the light treatment is intended to inhibit or deactivate microorganisms within the product or otherwise cause a photochemical reaction in the product. As used throughout this specification, the term “microorganism” is used generically and meant to include viruses, fungus, bacteria, contaminants and other living and non-living microorganisms that may be pathogenic or non-pathogenic. It is additionally noted that the term “a” appearing throughout is intended to mean “one or more” unless otherwise stated, i.e., the term “a” covers the singular and plural.

### Fluid Treatment System Using Light and Components

This section describes the structural components and uses of several different embodiments of treatment systems using a light treatment for the treatment of fluid products, for example, for treating a flowing fluid product with the light treatment.

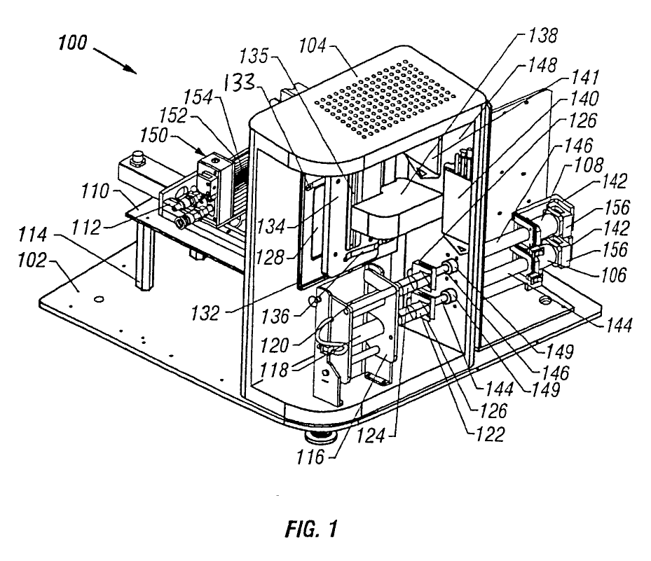

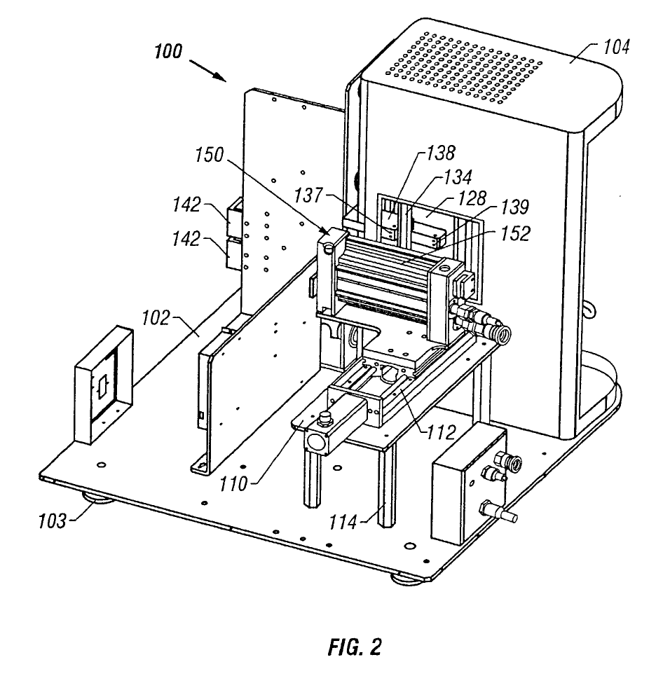

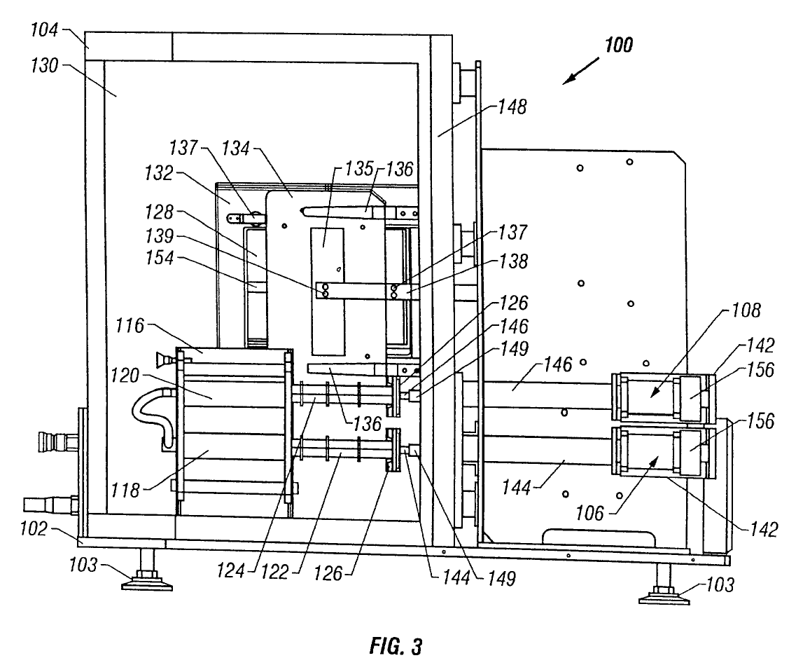

Referring first to FIGS. 1-3, several views are shown a fluid treatment system that uses a light source that emits a light treatment such as pulsed polychromatic light, for example, broad spectrum pulsed light (BSPL), according to one embodiment of the invention. FIG. 1 is a front perspective view, FIG. 2 is a rear perspective view, and FIG. 3 is front view of the fluid treatment system. Illustrated is the fluid treatment system 100 (generically referred to as a treatment system) including a base plate 102, support levelers 103, a treatment area enclosure 104, actuator assemblies 106 and 108 (also referred to generically as pumps), a lamp support plate 110, a linear slide servo drive 112 and support posts 114.

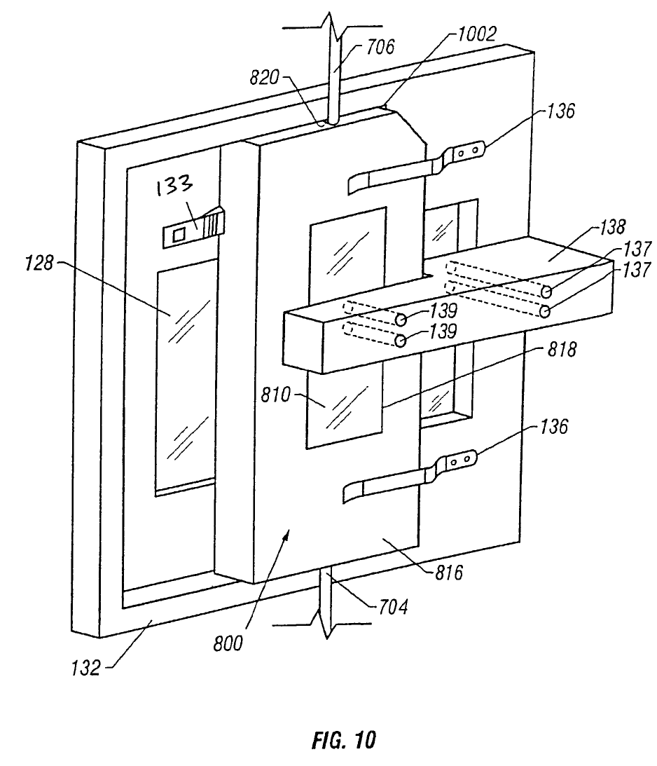

The actuator assemblies 106 and 108 are held in place by actuator assembly brackets 142 and each includes linear actuators 144 and 146 that extend through wall 148 at seals 149. At the end of the linear actuators 144 and 146 are respective brackets 126. The lamp support plate 110 holds a lamp assembly 150 including a reflector 152 and a light source 154 within profile of the reflector 152. It is noted that in preferred embodiments, the light source 154 is a pulsed light source, such as a flashlamp; however, in other embodiments, the light source 154 is a continuous wave light source (e.g., a UV lamp) or other pulsed light source operating at a single wavelength or operating within a range of wavelengths. It is noted that the light source 154 is partially viewable through window 128 in FIG. 3 and is also illustrated in FIG. 10. The treatment area enclosure 104 houses a treatment area from the rest of interior of the fluid treatment system 100. The treatment area enclosure 104 includes a syringe mount mechanism 116 that holds syringes 118 and 120 (also referred to generically as fluid containers 118 and 120) including syringe plungers 122 and 124. The syringe plungers 122 and 124 are adapted to be held by the brackets 126. A cartridge registration plate 132 is positioned within wall 130 of the treatment enclosure 104. A window 128 is formed within the cartridge registration plate 132. The cartridge registration plate 132 is adapted to positionally align and hold a cartridge 134 that in some embodiments, contains a treatment chamber. The cartridge 134 is held in place by cartridge lock clips 136 and a cartridge retaining clip 133. A process monitor housing 138 is positioned in front of a cartridge window 135 of the cartridge 134. The process monitor housing 138 includes process monitors 137 and 139 facing toward the cartridge 132. Note that the process monitors 137 and 139 are seen through the window 128 in FIG. 2 while the positioning of the process monitors 137 and 139 is seen through the process monitor housing 138 in FIG. 3. Also included are an effluent bag 140 and a sample bag 141 (each of which may be generically referred to as a fluid collectors or fluid containers).

The fluid treatment system 100 of this embodiment is designed to treat fluid products, including biological fluids, and their derivatives, e.g., blood, blood plasma, blood plasma derivatives, bioprocessing fluids and other fluid product, such as drugs and pharmaceuticals, especially bio-pharmaceuticals such as monoclonal antibodies, solutions such as a buffer, glucose and other sugar solutions, culture medias, as well as molecular biology and biochemistry reagents and other fluid product, etc., with light, for example, in this embodiment, with pulsed light. This light is generically referred to as a light treatment and is used, for example, to deactivate microorganisms including viruses, bacteria, fungus and other microorganisms that may be pathogenic or non-pathogenic.

In some embodiments, the light treatment is for the purpose of modifying or altering the product, or otherwise stimulating a change in the product or in a portion of the product. For way of example, the light treatment may be used for specific treatments including nucleic acid destruction, protein degradation, lipid degradation, carbon-carbon bond destruction of the product.

As used herein, the term fluid generally refers to liquids, gases, or solid materials that have the ability to flow; thus, the treatment system 100 may be used to treat a variety of flowable substances or products.

Generally, fluids are pumped from a fluid container (e.g., syringes 118 and 120), through a treatment chamber or treatment zone (such as formed within the cartridge 134) at a controlled rate while being illuminated with light from the light source 154, e.g., with pulses of light. The treated fluid product continues to flow to the effluent bag 140, while samples are collected in the sample bag 141 for testing, evaluation and use. Advantageously, since in one embodiment, the light treatment is pulsed light, the entire fluid treatment process is designed to be complete within several seconds, e.g., less than 10 seconds; however, this depends upon the flow rate, size of the fluid containers, etc. The fluid treatment system 100 is designed to be adjustable and scalable, for example, to a continuous flow system and, in some embodiments, includes a disposable treatment chamber or treatment zone.

In order to pump the fluid to be treated through the treatment chamber at a desired rate, a pump mechanism is provided. In this embodiment, fluids that are to be treated with light are contained with syringe 118, while syringe 120 contains another fluid which may be generically referred to as a buffer fluid, such as water for injection. Alternatively, the syringe 120 may contain more of the fluid product to be treated. These syringes 118 and 120 are loaded into the syringe mount mechanism 116 such that the body of syringes 118 and 120 are within the syringe mount mechanism 116 and the syringe plungers 122 and 124 extend out of the syringe mount mechanism 116 such that the head of the syringe plungers are captured by brackets 126. Actuator assemblies 106 and 108 are mounted such that they float freely in the axis of the syringe plungers 122 and 124, but in this embodiment, are retained within the actuator assembly brackets 142 and within wall 148 of the treatment area enclosure 104. Linear actuators 144 and 146 (of actuator assemblies 106 and 108) extend linearly through wall 148 at seals 149 and are rigidly mounted to the brackets 126 holding the syringe plungers 122 and 124, respectively. In this embodiment, the actuator assemblies 106 and 108 each have about a 5-inch stroke.

The actuator assemblies 106 and 108 are designed to operate independently from each other or together depending on the parameters set by the operator. Upon activation of either or both of the actuator assemblies 106 and 108, the respective linear actuators 144 and 146 begin to move (extend) toward the syringe plungers 122 and 124. However, in this embodiment, since the actuator assemblies 106 and 108 float freely within the actuator assembly brackets 142, the entire actuator assemblies 106 and 108 each move slightly away from the syringe plungers 122 and 124 until it contacts a load cell contained within a load cell block 156 coupled to the actuator assembly brackets 142. Once the load cell is contacted, it signals to the system controller that a fluid flow is being established. Further motion of the actuator assemblies 106 and 108 away from the syringe plungers 122 and 124 is now prevented since the load cell blocks 156 are held in place by the actuator assembly brackets 142; thus, the linear actuators 144 and 146 apply a force against the syringe plungers 122 and 124, respectively, being retained by the brackets 126. The linear actuators 144 and 146 move independently, together, or consecutively at a constant, variable, or other controlled rate set by the operator. The linear actuators 144 and 146 move the syringe plungers 122 and 124 into the syringes 118 and 120 forcing the fluid contained therein into tubing coupled to the cartridge 134. A flow rate is established by the linear velocity of the linear actuators 144 and 146. This rate is monitored by a linear encoder, e.g., a stepper drive, integrated into each of the linear actuators 144 and 146.

It is noted in this embodiment, that the “pump mechanism” includes the syringe mount assembly 116, brackets 126, actuator assemblies 106 and 108, actuator assembly brackets 142, load cell blocks 156, seals 149 and the linear actuators 144 and 146. However, one skilled in the art will recognize that a number of different pumping mechanisms may be used to produce a flow of fluid through the cartridge 134 at a specified rate. For example, pumps such as gear pumps, lobe pumps, pneumatic pumps, diaphragms, perostaltic pumps and gravity feeds may be used to effect a flow of fluid.

The fluids are forced to go through the tubing, which passes through a treatment chamber (also referred to as a treatment zone) contained within the cartridge 134. For example, in this embodiment, the fluid is forced through the cartridge 134 from bottom to top. In other embodiments, the fluid flow may be from top to bottom or side-to-side or other arrangement depending upon the configuration of the system. In one embodiment, as the fluid passes through the cartridge 134, the lamp assembly 150 including the light source 154 emits light, e.g., short duration pulses of light, to decontaminate the fluid. The light deactivates microorganisms contained within the fluid product.

It is noted that in some embodiments, the light source 154 is a flashlamp; however, in other embodiments, the light source 154 may comprise a light source other than the flashlamp 154, such as a continuous wave light source or a pulsed laser light source. Thus, the lamp assembly 150 may emit pulsed light or continuous light energy depending on the specific system design. Additionally, the fluence level of the emitted light is carefully selected to minimize protein damage, in the event the fluid is a sensitive biological fluid, e.g., a blood plasma derivative or other bioprocessing media. The treated fluid continues to flow out of the cartridge 134 and is collected in the effluent bag 140. During the course of a fluid treatment run, a sample of the treated fluid is collected in the sample bag 141. In this embodiment, the fluid in the sample bag 141 is retained for its intended use, such as, testing evaluation, or use in application. Thus, the contents of the effluent bag are typically discarded.

In operation, the fluid within syringe 120, for example, water for injection (WFI) or other buffer fluid or solution, such as saline, phosphate, etc., may be flowed prior to or at the same time as the fluid to be treated within syringe 118. Thus, the WFI may dilute the concentration of the fluid. Additionally, according to some embodiments, the WFI may be pumped through the cartridge 134 prior to pumping the fluid within syringe 118. As such, the WFI can be used to initialize the fluid treatment system and fill the fluid path to create backpressure and eliminate air bubbles, prior to flowing the actual fluids to be treated through. In some embodiments, as will be described in further detail below, the cartridge 134 contains a flexible treatment chamber such that the buffer solution initially flowed fills the fluid flow path which expands within the cartridge to a preset geometry defined by the interior structure of the cartridge 134, i.e., in this embodiment, the buffer solution is used to establish a treatment geometry of the treatment chamber or treatment zone, as well as establish the desired flow rate. Once established, the fluid to be treated (e.g., in syringe 118) is flowed through the flow path. Furthermore, the WFI is used to verify the operating parameters (such as fluid flow pressure, treatment geometry, desired light treatment fluence, for example) of the emitted light as set by the operator. Specific examples of using the buffer fluid to verify operating parameters are described more fully throughout this specification. Once the light treatment is verified and the fluid treatment system is operating correctly, then actuator assembly 106 is operated and the fluid to be treated (e.g., in syringe 118) is flowed through the flow path. After the initialization and a steady flow of the fluid to be treated is flowing through the system, the sample is collected in the sample bag 141. Advantageously, the use of WFI or a suitable buffer fluid is used to ensure uniform treatment of the fluid to be treated.

According to one embodiment, the lamp assembly 150 includes a light source 154 that provides pulsed polychromatic light, for example, broad spectrum pulsed light (BSPL), which illuminates and treats the fluid passing through the treatment chamber. BSPL is commonly produced by Xenon gas flashlamps, as known in the art. BSPL is pulsed light in the form of high-intensity, short duration pulses of incoherent polychromatic light in a broad spectrum, also referred to as broad-spectrum pulsed light (i.e. BSPL) or broadband pulsed light. For example, each portion of the fluid is illuminated by at least one, preferably at least two and most preferably at least three (e.g., 3, 5, 10, 15, 20, 30, 40 or more) consecutive short duration (e.g., less than about 100 ms, preferably about 150 μs or 300 μs) pulses of high-intensity (e.g., 0.001 J/cm² to 50 J/cm², e.g., 0.01 J/cm² to 1.0 J/cm², depending on the type of fluid being treated) incoherent polychromatic light in a broad spectrum (e.g., 170 nm to 2600 nm; i.e., 1.8×10¹⁵ Hz to 1.2×10¹⁴ Hz). However, such polychromatic light may comprise wavelengths within any subset of the range of 170 nm to 2600 nm (by filtering the emitted light, for example), e.g., the energy density or fluence of the pulsed light may be concentrated within wavelengths between 170 nm and 1800 nm, between 170 nm and 1000 nm, between 200 nm and 500 nm, or between 200 nm and 300 nm, for example. Furthermore, it has been found that certain biological fluids are most effectively treated with many short duration pulses of polychromatic light at relatively lower fluence levels. For example, in such cases, the fluid product is illuminated with about 20, 30 or 40 or more short duration pulses having intensities between 0.001 and 0.1 J/cm².

Broad-spectrum pulsed light (BSPL) described through this specification as a light treatment may also be referred to generically as “pulsed polychromatic light” or even more generically as pulsed light. Pulsed polychromatic light represents pulsed light radiation over multiple wavelengths. For example, the polychromatic light, whether pulsed or continuous wave, may comprise light having wavelengths between 170 nm and 2600 nm inclusive, such as between 180 nm and 1500 nm, between 180 nm and 1100 nm, between 180 nm and 300 nm, between 200 and 300 nm, between 240 and 280 nm, or between any specific wavelength range within the range of 170-2600 nm, inclusive. The choice of materials and/or spectral filters may be used produce a desired spectral range of the illumination. As is generally known, Xenon gas flashlamps produce pulsed polychromatic light having wavelengths at least from the far ultraviolet (200-300 nm), through the near ultraviolet (300-380 nm) and visible (380 nm-780 nm), to the infrared (780-1100 nm). In one example, the pulsed polychromatic light produced by these Xenon gas flashlamps is such that approximately 25% of the energy distribution is ultraviolet (UV), approximately 45% of the energy distribution is visible, and approximately 30% of the energy distribution is infrared (IR) and beyond. It is noted that the fluence or energy density at wavelengths below 200 nm is negligible, e.g., less than 1% of the total energy density. Furthermore, these percentages of energy distribution may further be adjusted. In other words, the spectral range may be shifted (e.g., by altering the voltage across the flashlamp) so that more or less energy distribution is within a certain spectral range, such as UV, visible and IR. In some embodiments it may be preferable to have a higher energy distribution in the UV range. It is further noted that pulsed polychromatic light may be produced by light sources other than Xenon gas flashlamps.

It is noted that although many embodiments of the invention utilize a light source 154 that provides a light treatment including pulsed polychromatic light (one example of which being BSPL), other embodiments of the invention use a light source 154 that provides pulses of monochromatic light, such as a pulsed laser emitting light at a specified wavelength. Thus, when referring to a fluid treatment system that uses “pulsed light”, it is meant that this pulsed light may be polychromatic or monochromatic pulsed light. It is also noted that although preferred embodiments of the invention utilize pulsed light, some embodiments utilize a light source 154 that provides continuous wave light, such as a continuous wave UV light, such as provided by Mercury gas lamps.

Thus, in general terms, the light source 154 of the fluid treatment system comprises a light source emitting light having at least one wavelength of light within a range between 170 nm and 2600 nm. For example, a pulsed polychromatic flashlamp (broad spectrum or narrow spectrum), a pulsed UV lamp, a pulsed laser, a continuous wave lamp, a continuous wave UV lamp, etc., could all serve as a light source 154 that may be used according to different embodiments of the invention.

Furthermore, in preferred embodiments, at least 0.5% (preferably at least 1% or at least 5%) of the energy density or fluence level of the pulsed polychromatic (or monochromatic) light emitted from the flashlamp 154 is concentrated at wavelengths within a range of 200 nm to 320 nm. The duration of the pulses of the pulsed light should be approximately from about 0.01 ms to about 100 ms, for example, about 10 μs to 300 μs.

In some embodiments, the fluence or intensity of the pulsed light should from 0.001 J/cm² to 50 J/cm², e.g., 1.0 J/cm² to 2.0 J/cm², depending on the fluid being treated. In embodiments where the fluid product to be treated is a blood plasma derivative or other bioprocessing fluid, the fluence of the pulsed light should be carefully selected to avoid extensive protein damage while at the same time deactivate microorganisms to a specified log reduction. For example, when treating biological fluids and their derivatives, such as blood, blood plasma, and blood plasma derivatives, the fluid product is illuminated with pulses of light having a fluence level preferably between 0.1 and 0.6 J/cm².

As a result of such illumination, microorganisms, such as viruses, fungus, bacteria, pathogens and other contaminants contained within the fluid are effectively deactivated up to a level of 6 to 7 logs reduction or more (i.e., a microbial reduction level that is commonly accepted as sterilization). Advantageously, it has been found by the inventors herein that the use of short duration, pulsed light, such as pulsed polychromatic light and broad-spectrum pulsed light (i.e., BSPL), effectively reduces the treatment time or exposure time of the treatment of fluids significantly (e.g., about 2 to 20 seconds compared to several minutes or more), increases the deactivation rate of microorganisms on or within target objects to a level commonly accepted as sterilization (about greater than 6 logs reduction of compared to 2-4 logs reduction), in comparison to known continuous wave UV fluid treatment systems.

In many applications, biological fluids are treated primarily to deactivate microorganisms without causing excessive protein damage. Thus, in these embodiments, the pulsed light treatment is configured to provide greater than 2 logs reduction, more preferably greater than 4 logs reduction and most preferably greater than 6 logs reduction is achieved with minimum protein damage. Although some of these deactivation levels fall short of what is accepted as sterilization, the pulsed light provides a significant advantage over a continuous wave UV treatment system in that microorganisms and other contaminants are effectively deactivated at desired log reduction rates with minimum protein damage in a short period of time. Furthermore, the use of BSPL using Xenon flashlamps completely eliminates the problem of Mercury contamination due to broken Mercury lamps that may be encountered in such a continuous wave UV fluid treatment device, since Xenon is an inert gas which is harmless if exposed due to leakage or breaking of the Xenon flashlamp. Variants of Xenon flashlamps, such as those described in U.S. Pat. No. 6,087,783 of Eastland, et al., entitled METHOD AND APPARATUS UTLILIZING MICROWAVES TO ENHANCE ELECTRODE ARC LAMP EMISSION SPECTRA, issued Jul. 11, 2000, which is incorporated herein by reference, may also be used as an appropriate light source for the fluid treatment system 100.

Several apparatus designed to provide high-intensity, short duration pulsed incoherent polychromatic light in a broad-spectrum are described, for example, in U.S. Pat. Nos. 4,871,559 of Dunn, et al., entitled METHODS FOR PRESERVATION OF FOODSTUFFS, issued Oct. 3, 1989; 4,910,942 of Dunn, et al., entitled METHODS FOR ASEPTIC PACKAGING OF MEDICAL DEVICES, issued Mar. 27, 1990; 5,034,235 of Dunn, et al., entitled METHODS FOR PRESERVATION OF FOODSTUFFS, issued Jul. 23, 1991; 5,489,442 of Dunn, et al., entitled PROLONGATION OF SHELF LIFE IN PERISHABLE FOOD PRODUCTS, issued Feb. 6, 1996; 5,768,853 of Bushnell, et al., entitled DEACTIVATION OF MICROORGANISMS, issued Jun. 23, 1998; 5,786,598 of Clark, et al., entitled STERILIZATION OF PACKAGES AND THEIR CONTENTS USING HIGH-DENSITY, SHORT-DURATION PULSES OF INCOHERENT POLYCHROMATIC LIGHT IN A BROAD SPECTRUM, issued Jul. 28, 1998; and 5,900,211 of Dunn, et al., entitled DEACTIVATION OF ORGANISMS USING HIGH-INTENSITY PULSED POLYCHROMATIC LIGHT, issued May 4, 1999, all of which are assigned to PurePulse Technologies of San Diego, Calif. and all of which are incorporated herein by reference.

As partially shown in FIG. 3 and as more clearly illustrated in FIG. 10, the light source 154 is oriented transverse to the direction of the fluid flow. However, the light source 154 could be arranged in a different orientation, depending on the specific system configuration. Furthermore, although only one light source 154 is illustrated, more than one light source 154 could be used (e.g., one or more lamps or other light sources), depending on the length flow path, the flow rate and other requirements of the system.

In order to ensure that the light treatment, e.g., pulsed light, emitted from the lamp assembly 150 provides the proper treatment levels, such as the proper fluence and the proper spectrum, process monitors 137 and 139 are located within the process monitor housing 138. These process monitors 137 and 139 may comprise one or more of several types of optical detectors or optical monitoring devices, such as photodetectors, photodiodes, fiber optic probes, calorimeters, joulemeters, photomultiplier tubes (PMTs), cameras, charged coupled device (CCD) arrays, and inputs to a spectrometer, such as a spectroradiometer. These process monitors 137 and 139 may also be thermodetectors, such as thermocouples, thermopiles, calorimeters, and joulemeters. In one embodiment, one or more of process monitors 137 and 139 are photodetector devices that receive light emitted directly from the light source 154, as well as receive light received or transmitted through the product to be treated and the cartridge 134. Furthermore, in some embodiments, one or more of the process monitors 137 and 139 detect the ultraviolet (UV) portion of the light, while others of process monitors 137 and 139 detect full spectrum light emitted from the light source. For example, in one embodiment, the process monitors comprise fiber optic probes that are coupled to a two-channel spectrometer, such that one channel is used to measure the UV content of the light treatment and the other channel is used to measure the visible spectrum of the light treatment.

As will be described below, the cartridge 134 includes light transmissive plates or windows (which may be generically be referred to as “light transmissive support structures”) on both sides such that the light treatment transmits through the cartridge 134 to the treatment chamber inside. At least a portion of the light treatment also transmits through the treatment chamber and the fluid product through the window 135 in the cartridge 134, such that the process monitors 139 of the process monitor housing 138 can detect the light penetrating the fluid, which is also helpful to determine the absorption of light by the fluid. Additionally, process monitors 137 detect the light emitted directly from the light source 154. For example, see FIGS. 10 and 15A, and 16A for further details. It is noted that generally when referring light transmissive components, such as treatment chambers, support structures, products to be treated, it is meant that such items are transmissive to at least 1% of light having at least one wavelength within a range of between 170 nm and 2600 nm.

The fluence level is generally adjustable by adjusting the voltage across the light source 154, e.g., flashlamp; however, it has been found that these adjustments affect the fluence or intensity profile of the emitted light over the given spectrum, i.e., a change in the voltage across the light source 154 non-uniformly changes the fluence across the given spectrum. Furthermore, the fluence received at the cartridge 134 is also adjustable by linearly adjusting the distance of the lamp assembly 150 (and thus, the light source 154) from the cartridge 134. This provides for a uniform adjustment of the fluence without affecting its spectral intensity across the emitted spectrum. Thus, the entire lamp assembly 150 moves linearly on the lamp support plate 110 as driven by the linear slide servo drive 112. In effect, the distance from the light source 154 to the treatment chamber or treatment zone is adjustable. In many embodiments, the fluence is adjustable during the treatment process. In one embodiment, the lamp assembly may be adjusted as much as 13 inches from the window 128 of the treatment area enclosure 104. Thus, as measured by the process monitors 137 and 139, the fluence of the emitted light is adjustable between 0.1 and 0.5 J/cm², in one embodiment, depending on the position of the lamp assembly 150 on the linear servo drive 112. Additionally, this range could be larger or smaller depending on the design and shape of the reflector 152, or modification of the size or energy of the light source 154 such as would be obvious to those skilled in the art.

It is also noted that in some embodiments, the adjustment of one or more system parameters, such as fluence, fluence profile over a desired spectrum, distance of the light source 154 to the treatment chamber, voltage across the light source 154, etc., may be automatically made in response to measurements provided by the process monitors 137 and 139. In such embodiments, a controller utilizes the measurements of the process monitors 137 and 139 and other system and light treatment parameters to automatically determine and cause the appropriate adjustments to be made in order to result in the desired system parameters as input by the user. Such control features are described more fully throughout this specification.

As will be described below, in some embodiments, the cartridge 134 contains the treatment chamber. All of the components of the fluid path, including the treatment chamber are designed to be easily removable and disposable. For example, the syringes 118 and 120, the treatment chamber, the effluent bag 140, the sample bag, and all of the tubing connecting these components are disposable. This eliminates the requirement of “cleaning” each of these components when switching between different runs of fluids. In some embodiments, the entire fluid flow path can be installed and removed as a sealed fluid flow path.

The fluid treatment system 100 is designed for adjustability of the light treatment. Such adjustability may be automatic or manual. For example, the fluence of the light treatment, the flow rate of the fluid, and the thickness of the fluid as its being treated are all adjustable. According to one example, the fluid treatment system can provide light treatment of up to 6 J/cm², and up to 10 flashes at a flow rate of 1 liter/minute. However, all of these parameters are designed to be adjustable depending on the requirements of the system and operator. Thus, in another example, with adjustments to the treatment chamber, the flow rate is scalable to 11 liters/minute or higher with similar treatment parameters. For example, the treatment can also be scaled to treatment at greater than 10 pulses (i.e., 20, 30, 40 or 50 pulses, etc.) by reflector/lamp modifications (as noted above) and/or by increasing the pulse generator power. However, it is noted that various adjustments in the pump rate, the flash rate and the relative size of various components in the fluid flow path, may be made depending on the implementation. The operator can vary the flow rate and the flash rate to any of a number of different settings. Furthermore, with minor modifications, additional, alternate pumping devices pump fluids from larger fluid sources or containers that are coupled through the cartridge 134, rather than from syringes 118 and 120, for a continuous flow and fluid treatment system.

Furthermore, the fluid treatment system 100 is adapted to be coupled to a computer/controller, which provides the electronic control and processing as well as the user interface for the fluid treatment system 100. For example, the user enters operating parameters, such as flow rate, flash rate, fluence, etc., while a computer-based control system receives measurements taken in operation and automatically makes adjustments to ensure that these parameters are met. In embodiments using a pulsed light treatment, such as BSPL, an energy storage and pulse generating device is also coupled to the fluid treatment system and coupled to the flashlamp. This is more fully described with reference to FIG. 41.

Additionally, in embodiments using pulsed light sources, such as Xenon gas flashlamps, it is known that Xenon gas flashlamps generate a significant amount of heat during extended use. However, generally, the length of time for most fluid runs using this embodiment will be very short in duration, thus, cooling means are not required. However, in a scaled up version of the fluid treatment system that is designed to run continuously and pumps fluid from a continuous source or container, cooling means are important.

In some embodiments, it is noted that the treatment system 100 is used as an experimental tool that is adjustable in a plurality of ways and used to determine an optimal set of operating parameters for a given product to be treated with light. For example, a given product may be tested using the treatment system, each test varying one or more of the system or light treatment parameters. The treatment system stores measurements for each test for comparison. After many tests using the product, all of the results are analyzed to determine what is the optimal set of system and light treatment parameters for the given product. For example, after a battery of tests, it is determined that the given product is most effectively treated (i.e., the highest deactivation rate with an acceptable level of damage to the product) at a certain flow rate, exposure (e.g., flash rate, number of flashes, fluence level, spectrum), fluid concentration, geometry of treatment chamber, etc. Once these optimal parameters are known, then a simplified, production scale treatment system specifically tailored to the product to be treated can be designed and produced. Further details are described throughout this specification.

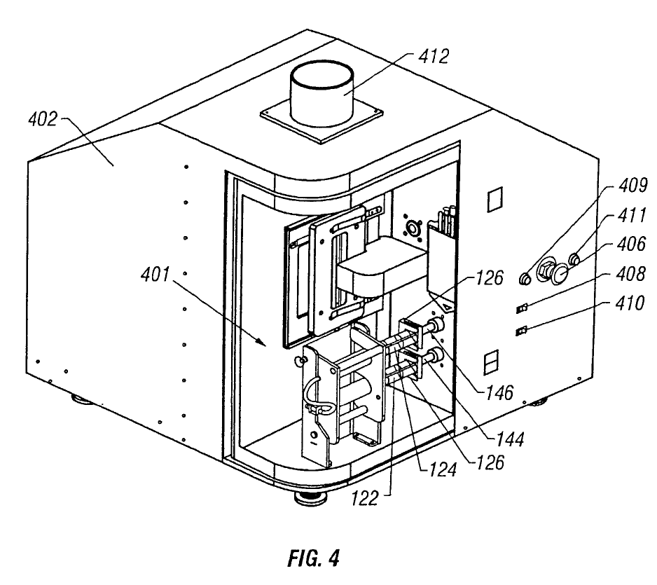

Referring next to FIG. 4, an external view is shown of the fluid treatment system of FIGS. 1-3. An enclosure 402 surrounds the fluid treatment chamber 100 such that the lamp assembly, actuator assemblies, and other electronics and controls are not accessible to the user. The enclosure 402 includes a treatment area opening 404, which allows access to the treatment area 401 including the syringes 118 and 120, the cartridge 134, the sample bag 141 and the effluent bag 140. A treatment area door (not shown) is also provided to seal off the treatment area 401 during use. Also, the treatment area 401 is sealed from the rest of the interior of the fluid treatment system 100 by the treatment area enclosure 104. Thus, any fluid spills or other accidents are confined to the treatment area 401, and will not contaminate the rest of the interior of the fluid treatment system 100. Additionally, the treatment area door is opaque to prevent the light treatment from escaping the fluid treatment system during use. The enclosure 402 also includes user controls, such as an emergency power off switch 406 and indicator lights 409 and 411. Additionally, also provided are toggle buttons 408 and 410, which are used to adjust the linear position of the linear actuator 144 and 146 either left or right in order that they can properly retain the plunger heads of the syringe plungers 122 and 124. This is because, the heads of the syringe plungers 122 and 124 extend a variable distance from the body of syringes 118 and 120. Since the plunger heads are to be held by the brackets 126 at the end of the linear actuators 144 and 146, the toggle buttons 408 and 410 move the bracket to the left or right. Thus, the plunger heads will align within the brackets 126. Furthermore, a fan cover 412 is also shown. The fan cover 412 heat and/or ozone to be pulled from the interior of the fluid treatment system to the exterior by a fan underneath the fan cover 412.

Referring next to FIG. 5, a perspective view is shown of the syringe mount assembly 116 of FIGS. 1-3 according to one embodiment of the invention. In order to load syringes, e.g., syringes 118 and 120 of FIG. 1, a syringe pump mount plate 502 (also referred to generically as a fluid container holder) rotates outward relative to a syringe pump mount bracket 504 about bar 506. The syringe pump mount plate 502 includes slots 508 and 510 for receiving syringes 118 and 120, respectively. Once positioned in the slots 508 and 510, the syringe pump mount plate 502 is rotated back flush with the syringe pump mount bracket 504. Pushpin 512 is inserted through hole 514 of the syringe pump mount bracket 504 and hole 516 of the syringe pump mount plate 502 to lock the syringe mount assembly 116 in position.

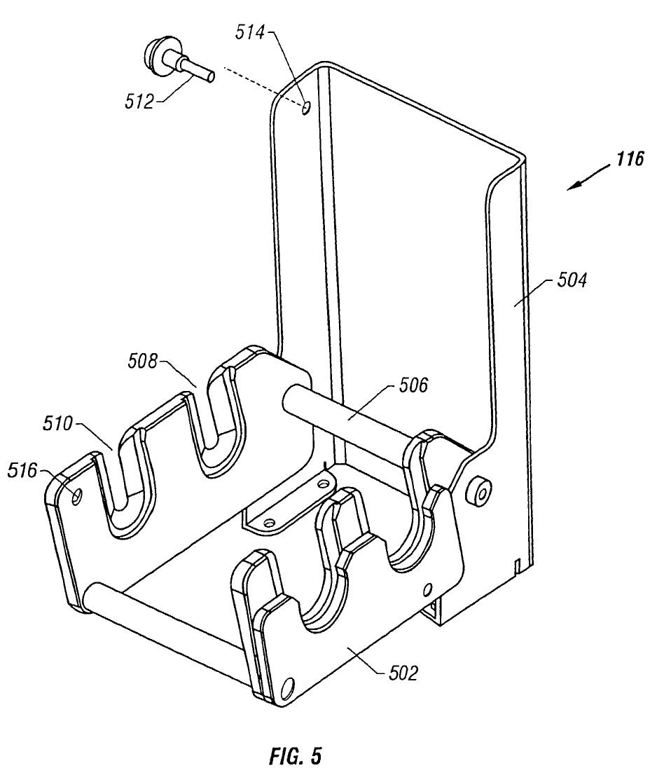

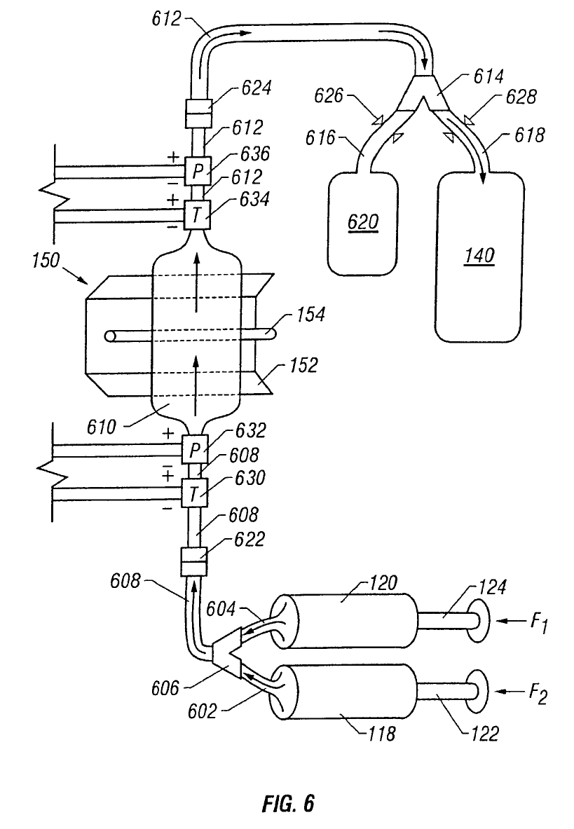

Referring next to FIG. 6, a schematic view is shown of one embodiment of the fluid flow path components of the fluid treatment system of FIGS. 1-3. Shown are the syringes 118 and 120 (each of which may be generically referred to as “fluid container portions” of a fluid flow path for use in a generic fluid treatment system) including tubes 602 and 604, respectively. Tubes 602 and 604 are connected at Y-fitting 606. Alternatively, Y-fitting 606 is a T-fitting, as is illustrated in FIGS. 1 and 3. A T-fitting is preferable since the T-fitting can be directly coupled to one of the syringes (e.g., syringe 118 of FIG. 1) such that tube 602 can be eliminated or its length shortened. Tube 608 (also referred to as the supply conduit or input conduit) couples the Y-fitting 606 (or alternatively, T-fitting or other fitting) to an input of a treatment chamber 610. The treatment chamber 610 may also be referred to generically as a “treatment chamber portion” or “treatment zone” of a fluid flow path. An output of the treatment chamber 610 is coupled to tube 612 (also referred to as the output conduit), which splits at Y-fitting 614 into tubes 616 and 618, which are connected to a sample bag 141 and the effluent bag 140, respectively. The sample bag 141 and the effluent bag 140 can be generically referred to as fluid container portions or fluid collector portions of the fluid flow path. In order to easily connect the treatment chamber 610 in-line, quick disconnect 622 is optionally provided in tube 608 and quick disconnect 624 is provided in tube 612. These quick disconnects 622 and 624 may be any quick disconnects as known in the art, such as CDC quick disconnects produced by Colder Products Company of St. Paul, Minn., USA or other luer quick disconnects available from Value Plastics, Inc. of Fort Collins, Colo., USA, as known in the art. Furthermore, solenoid valves 626 and 628 (e.g., pinch valves) control the flow of fluids into the sample bag 141 and the effluent bag 140, respectively.

Additionally, in order to monitor the pressure and temperature of the fluid flow, pressure transducer 632 and thermocouple 630 are coupled the input of the treatment chamber 610, e.g., coupled to tube 608.

Additionally, pressure transducer 636 and thermocouple 634 are coupled at the output of the treatment chamber 610, e.g., coupled to tube 612. These pressure transducers and thermocouple provide an electrical signal to be transmitted to a process controller of the system. Thus, the system is able to measure the pressure of the fluid flow at the input and the output of the treatment chamber, as well as monitor any changes in the temperature of the fluid flow due to the light treatment. It is noted that Xenon gas flashlamps and other pulsed light sources may generate significant heat, which may increase the temperature of the fluid. Thus, depending on the sensitivity to heat of the fluid being tested, the fluence of the light source 154 may be adjusted (e.g., by adjusting the distance between the light source 154 and the treatment chamber 610) in response to the measurements taken by the pressure transducers and thermocouples. It is noted that the pressure transducers 632 and 636 and thermocouples 630 and 634 may also be referred to generically as process monitors, since they are used to monitor the fluid flow. In further embodiments, flow rate sensors or monitors may be placed at the input and the output of the treatment chamber to monitor the flow rate of the fluid in addition to or in replacement of one or more of the pressure transducers 632, 636 and thermocouples 630, 634. It is noted that in some embodiments, it is important to maintain a constant flow rate through the treatment chamber. Thus, such flow rate monitors are used to ensure that the measured flow rate is substantially equal to the desired or set flow rate. It is further noted that cooling mechanisms may be used to cool the light source 154 and/or cool the product being treated. For example, the light source 154 may be cooled by flowing a cooling medium through a tube or sheath surrounding the light source 154. The product may be cooled by circulating a cooling medium against the treatment chamber 610, such as a fan or other refrigeration device positioned against or near the treatment chamber 610.

In operation, syringe 118 contains the fluid or fluid product to be treated with the light treatment, e.g., contains inoculated or contaminated fluid, while syringe 120 contains either uninoculated fluid or WFI (water for injection), or other buffer fluids or solutions as described above. Actuator devices or pumps (e.g., actuator assembly 106 including linear actuator 144, or other pumping devices) operate independently or at the same time to apply forces, e.g., F1 and F2, to the plungers 122 and 124 of the syringes 118 and 120. This causes the fluids within one or more of the syringes 118 and 120 to be forced into the tubes. For example, the fluid in syringe 118 is forced into tube 602, through Y-fitting 606, through tube 608 and through the treatment chamber 610 or treatment chamber at a desired flow rate. The flow rate is dictated by the syringe barrel diameter and the linear actuator velocity, which is set by the operator and coordinated with the flash rate of the flashlamp 154. These actuator assemblies, and thus the flow rate, are under the control of electronics within the fluid treatment system.

As the fluid passes through the treatment chamber 610, the fluid is exposed to the light treatment, e.g., the fluid is exposed to one or more flashes of pulsed light, emitted from light source 154. Also included is the reflector 152 positioned behind the light source 154 and is shaped to project a fluence pattern toward the treatment chamber 610. In one embodiment, the light source 154 is a Xenon gas flashlamp which emits BSPL, as described above. The fluence of the light received at the treatment chamber 610 is adjustable by adjusting the power to the light source 154 and/or by adjusting the linear distance between the light source 154 and the treatment chamber 610. It is noted that a linear distance adjustment is preferred since it provides for a uniform adjustment of the fluence across the full spectrum of the emitted light. It is noted that although only one light source is shown, the system may include more than one light source or lamp.

The fluid continues to flow out of the treatment chamber 610, through tube 612, Y-fitting 614, and into one or both of the sample bag 141 and the effluent bag 140, via tubes 616 and 618, respectively. The fluid flow into the sample bag 141 and effluent bag 140 is controlled by the solenoid valves 626 and 628. During most of the fluid run, solenoid valve 628 is open and solenoid valve 626 is closed such that the fluid is directed to the effluent bag 140. Thus, the effluent bag 140 contains a mixture of treated, e.g., decontaminated fluid product and fluids from syringe 120, e.g., water for injection or other solutions. Alternatively, the effluent bag 140 may contain only the fluid to be treated in the event both syringes 118 and 120 contain the same fluid. In order to collect a clean, usable sample, solenoid valve 626 is opened while solenoid 628 is closed to collect a predetermined amount (set by the operator) of fluid within the sample bag 141 for testing and evaluation or use.

Generally, the treatment chamber 610 may be a flexible or rigid structure having a given geometry. According to several embodiments, the treatment chamber 610 is generally a substantially flat sheet-like treatment chamber. The treatment chamber may be disposable or reusable. The treatment chamber 610 may also be a flexible bag-like material or a rigidly shaped material. In some embodiments, the treatment chamber is a substantially tubular structure that may be flexible or rigid. In embodiments where the treatment chamber is a flexible material, the buffer fluid is useful to establish a geometry of the treatment zone. For example, as the buffer fluid is flowed through the treatment chamber, the buffer fluid causes the treatment chamber to expand under the pressure of the fluid until it is “inflated”. At this point, the geometry of the fluid flow path and treatment zone is “established”. In some embodiments, the treatment chamber expands into plates or other structure that define one or more dimensional boundaries of the fluid flow path or treatment zone. Once the geometry, pressure and flow rate are established, the fluid product to be treated is flowed therethrough. This helps to ensure uniform treatment of the fluids to be treated.

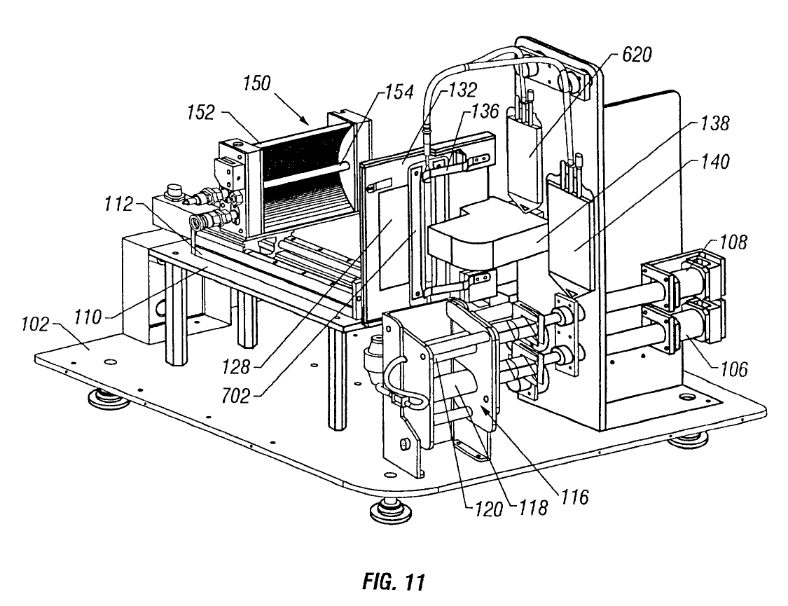

In some embodiments, the treatment chamber 610 is generally held within a cartridge, such as shown in FIGS. 1-3; however, in alternate embodiments, the cartridge is not required, such as shown in FIG. 11. Thus, in the alternate embodiments, the treatment chamber is simply positioned in front of the lamp assembly 150 or in front of the light source of a treatment system generically for treatment. In embodiments using a cartridge, the cartridge restrains the treatment chamber 610 between two light transmissive support structures or plates separated by a specified distance. Thus, in some embodiments, the flow of fluid within the treatment chamber 610 is a substantially flat laminar flow having an adjustable thickness and an adjustable width. However, it is noted that the flow may be characterized as flat, laminar, uniform, tubular, turbulent or any other flow as understood in the art. In some embodiments, the thickness of the treatment chamber 610 is adjustable by using an adjustment mechanism that varies the specified thickness (this is further described later in the description). The width is adjustable in the selection of the appropriate treatment chamber. For example, the operator may have a choice between many differently sized treatment chambers having different widths depending on the manufacturing specifications.

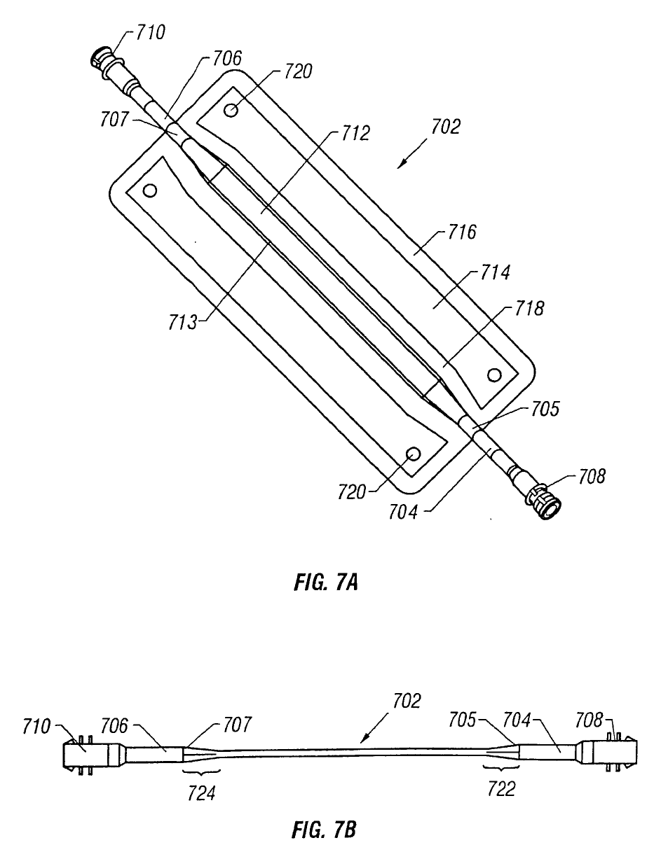

Generally, the treatment chamber 610 is light transmissive. In some embodiments, at least a portion of the treatment chamber is transmissive to at least 1% of light having at least one wavelength between 170 and 2600 nm. For example, the treatment chamber 610 is made of materials transmissive at least portions of the light emitted by the light source 154, e.g., FEP (flourinated ethylene-propylene perfluoro (ethylene-propylene)), EVA (ethylene vinyl acetate), PTFE (polytetrafluoroethylene), PFA (perfluoro(alkoxy alkane)), ethyl vinyl alcohol, polyvinylidene fluoride (PVDF), polyvinyllidine chloride (PVDC): Saran, and polyamides, such as nylon and polychlorotrifluoroethylene (PCTFE): Aclar. Thus, in some embodiments, the treatment chamber 610 is made of materials such as polymers, polyolefins, fluorinated polymers, halogenated polymers, polyamides, nylons, plastics, or combinations thereof. Various embodiments of the treatment chamber 610 and the cartridge are described further below, for example, with reference to FIGS. 7A, 7B, 12, 13, and 14, although it is appreciated that the treatment chamber may take many forms other than those specifically described in FIGS. 7A, 7B, 12, 13 and 14.

In one embodiment, the entire fluid flow path is sealed and removable from the fluid treatment system. In this embodiment, the fluid flow path may be defined as having a first fluid container portion, e.g., one or both of the syringes 118 and 120, a treatment chamber portion, e.g., the treatment chamber 610, and a second fluid container portion, e.g., one or both of the sample bag 141 and the effluent bag 140. The first fluid container portion contains the fluid to be treated with the light treatment. The fluid in the first fluid container portion is flowed through the treatment chamber portion and illuminating with light. The treated fluid is collected in the second fluid container portion. Advantageously in this embodiment, the first fluid container portion is sealingly coupled to an input of the treatment chamber portion (e.g., using flexible tubing and connectors) and the second fluid container portion is sealingly coupled to an output of the treatment chamber portion (e.g., using flexible tubing and connectors). In this embodiment, the entire fluid flow path may be pre-sterilized and contain the fluid to be treated. The entire fluid flow path may be inserted into the fluid treatment system (e.g., the fluid treatment system 100) and removed once the light treatment is completed. Once the treated fluid or treated sample is removed, the entire fluid flow path may then be discarded and replaced with another fluid flow path; thus, eliminating the need to sterilize the fluid flow path after each use.

Furthermore, in some embodiments, many components of the fluid flow path are designed of inexpensive materials, such as plastics, nylons, polymers, or combinations thereof. Many of these components may also be made of generally flexible materials. It is noted that although the entire fluid flow path may be made sealed and removable from the fluid treatment system in some embodiments, the fluid flow path is not required to be installed as a sealed fluid flow path. For example, one or more components may be inserted separately into the fluid treatment system and then coupled and sealed together. In another example, the entire fluid flow path may be coupled and sealed together and then inserted into the fluid treatment system.

Furthermore, a sealed fluid flow path may be embodied in any number of geometries and includes for example, a first container portion that contains a fluid to be treated, a treatment chamber portion coupled to the first container portion that is adapted to have the fluid flowed therethrough and a second container portion coupled to the treatment chamber portion that is adapted to receive the fluid that is flowed through the treatment chamber portion. The fluid may be flowed through the treatment chamber portion using a pump or other device or by any means to cause the fluid to flow from one portion to another, for example, even through the use of gravity. While the fluid is being flowed through the treatment chamber portion, the fluid is treated with light from the light source. The different portions may be coupled to each other via tubing or connectors as illustrated, or in other embodiments, the first container portion, the second container portion and the treatment chamber portion are one integral structure. Furthermore, in some embodiments, the sealed fluid flow path may be made of any of the materials listed above and may be flexible or rigid. It is also noted that in some embodiments, the fluid to be treated may initially not be present in the first container portion, but is injected or inserted into the first container portion prior to being flowed through the treatment chamber portion. It is also noted that the flow of the fluid through the treatment chamber 610 may take a variety of forms. For example, depending on the geometry of the treatment chamber, the fluid may flow therethrough in a laminar flow, a flat flow, a tubular flow, a uniform flow, a non-uniform flow, and a turbulent flow to allow mixing, etc.

In many embodiments, the buffer fluid, e.g., WFI, within syringe 120 is used to initialize the fluid treatment system and fill the fluid path to create back pressure and eliminate air bubbles, prior to flowing the actual fluid product to be treated with the light treatment. Additionally, the buffer fluid may dilute the concentration of the fluid. Furthermore, in some embodiments, the buffer fluid is used to verify the light treatment parameters, such as to verify the fluence level of the light source or to verify the cleanliness of light transmissive system components. Once the light treatment and system cleanliness is verified and the fluid treatment system is operating correctly, then actuator assembly 106 is operated and the fluid to be treated (e.g., in syringe 118) is flowed through the flow path.

Referring next to FIG. 7A, a perspective view is shown of one embodiment of the treatment chamber of FIG. 6. Illustrated is the treatment chamber 702 including an input tube 704 (or supply conduit) coupled to an input port 705, an output tube 706 (or output conduit) coupled to an output port 707, each having a respective quick disconnect 708 and 710. The input tube 704 and the output tube 706 are round tubes coupled to the input and output ports 705 and 707. The input and output ports 705 and 707 taper into a flow chamber 712 of the treatment chamber 702. It is noted that in preferred embodiments, the taper from the input and output ports 705 and 707 to the flow chamber 712 should be designed to uniformly translate the generally circular cross sectional flow of the fluid through the tubes to the substantially laminar flow profile through the flow chamber 712. This is further illustrated with reference to FIG. 7C. However, it is noted that the taper from the input and output ports 705 and 707 may be made to designed to minimize dead spots or stagnation and to generally maintain a substantially uniform flow. In other embodiments, the flow through the flow chamber 712 may be designed to be a turbulent flow such that the fluid is mixed as it is flowed through the flow chamber.

The flow chamber 712 extends from the input port 705 to the output port 707. It is noted that the flow chamber 712 may be generically referred to as a treatment zone or portion of the fluid flow path. The body portion 714 of the treatment chamber 702 is generally formed using multiple sheets of a light transmissive material, such as a polymer, polyolefin, fluorinated polymer, halogenated polymer, polyamide, nylon, plastic, or combinations thereof. Thus, by way of example, FEP, EVA, PTFE, PFA, PVDF and PCTFE may be used for the body portion 714. These two sheets are placed on top of each other and sealed together at the exterior edges 716 and at the boundary 718 to the flow chamber 712. For example, the sheets of material are welded (e.g., radio frequency (RF) welded), or other wise bonded to each other to form the treatment chamber 702. Thus, the treatment chamber 702 is generally flat and flexible, having a flow chamber 712 formed therethrough.

In some embodiments, prior to bonding or attaching the sheets together, a slight preform 713 is formed in each sheet of material proximate to the boundary of the flow chamber 712. The preform 713 may be a slight bend or other deforming feature. This preform allows the flexible sheets to form the flow chamber more naturally without causing creasing along the edge of the flow chamber as the fluid fills up and passes through the flow chamber 712. However, even with the preform, the flow chamber is substantially flat without the presence of a fluid flowing therethrough.

In operation, the fluid is forced through the input port 705 into the flow chamber 712 and out through the output port 707 at a controlled rate. As the fluid product flows through the flow chamber 712, the volume of the flow chamber expands, i.e., the flow chamber fills up or inflates to form a generally flattened elliptical tubular structure. As such, the fluid flowing through the flow chamber 712 establishes a flow geometry of the flow chamber 712. However, the thickness of the flow chamber 712 is generally not uniform across the width of the flow chamber 712. For example, the flow chamber 712 is slightly wider at the center in comparison to the edges across the width of the flow chamber 712. Additionally, the thickness of the material of the body portion 714 that forms the flow chamber 712 is designed to be able to withstand the pressure of the fluid as it is pumped or other wise forced through the flow chamber 712.

In preferred embodiments, the treatment chamber 702 is positioned against a structure that is at least partially light transmissive, e.g., positioned within the cartridge as described above. In order to align the treatment chamber 702 within the cartridge, holes 720 are punched in the body portion 714 through which alignment pins of the cartridge or other retaining assembly pass. It is noted that these holes 720 may be referred to generically as “alignment features” and the alignment pins may be referred to generically as “corresponding alignment features”. Other types of alignment features and corresponding alignment features may include tapers, wedges, ridges, key in slots, etc.

As described further below, several embodiments include one or more light transmissive support structures, e.g., plates or windows, positioned against the treatment chamber 702. The one or more support structures effectively define one or more dimensional boundaries of the flow chamber 712; thus, the one or more light transmissive support structures define one or more dimensional boundaries of the treatment zone or treatment volume. For example, if the treatment chamber 702 is held against a single plate or window, the single plate or window defines one dimensional boundary of the flow chamber 712. In the case of two plates or windows, the treatment chamber 702 is sandwiched between the two plates, i.e., the two plates define two dimensional boundaries of the flow chamber 712. These plates or windows effectively flatten out the flow chamber 712 once the flow chamber is filled with fluid to provide a laminar fluid flow through the flow chamber 712 for substantially uniform light treatment. Thus, according to several embodiments, the fluid flow through the flow chamber 712 establishes a geometry of the flow chamber 712 by expanding the flow chamber into the dimensional boundaries of the surrounding support structure, e.g., plates. In preferred embodiments, a suitable buffer fluid is initially flowed through the flow chamber 712 to establish the flow geometry, flow rate, etc. before flowing the fluid to be treated therethrough. Depending on the shape of the one or more plates or windows, the thickness therebetween may or may not be uniform; thus, the fluid flow may or may not have a uniform thickness throughout the length of the flow chamber 712. The distance between the two plates or windows can be controlled, such that the flow chamber 712 has an adjustable fluid thickness. In some embodiments, the fluid flow is substantially uniform across its width and along the length of the flow chamber 712. It is noted that the one or more support structures may comprise flat or curved plates, and at least portions of which may be transmissive to at least a portion of the light treatment. In embodiments where the plates are curved, the curvature of the two plates may be the same or different depending upon the embodiment. It is noted that the one or more plates may be referred to generically as a “treatment chamber support structure” or “treatment zone support structure” that defines one or more dimensional boundaries of the flow chamber 712 or treatment zone or treatment volume. It is also noted that in alternate embodiments, the treatment chamber 702 itself may be positioned in front of one or more light sources without necessarily being positioned within or against one or more light transmissive support structures, e.g., plates or windows. In some embodiments described below, the treatment chamber is held within a specially designed cartridge. In some embodiments, the treatment chamber 702 resembles a liner-like structure to the support structure (e.g., the one or more plates or windows or the cartridge).

Advantageously, the treatment chamber 702 is designed to be light transmissive to at least a portion of the light emitted from the light source 154. Furthermore, the treatment chamber 702 is easily manufactured such that it is disposable after use. The treatment chamber 702 is simply removed at the quick disconnects 708 and 710 and replaced for the next fluid treatment. This eliminates the requirement of having to clean out or flush the treatment chamber 610 when switching between different types or runs of fluids. In some embodiments, the entire fluid flow path is disposable. For example, the treatment chamber 702 along with the syringes, the tubing, and the sample bag and the effluent bag are all removed and replaced after each use. Advantageously, there is not need to clean out these components since they are replaced by pre-sterilized components for the next run.

This treatment chamber is a departure from known light treatment devices. In known light treatment fluid devices, a volume is defined within the device that is a treatment volume. The fluids, typically water, are passed through the treatment volume at a low flow rate and treated with light, such as continuous wave ultraviolet light. The treatment volume is defined by a rigid container that allows the fluid to flow therethrough. This conventional treatment chamber is a rigid structure that is designed for multiple uses and must be cleaned out prior to treating different fluids. Such treatment chambers are commonly made of a rigid quartz, or similar light transmissive, material. Manufacturing a quartz container can be expensive and time consuming. Thus, replacing such a quartz material treatment chamber after each use would be prohibitively expensive. Furthermore, such treatment chambers are rigid in order to adequately contain the fluid product.

In contrast, the treatment chamber of this and other embodiments of the invention is disposable and flexible. The dimensional boundaries are not rigidly set and may be affected by positioning the treatment chamber against the appropriate support structure. Applicants are not aware of other flexible treatment chambers. A sealed flexible bag containing a fluid may be treated within a treatment device; however, the fluid is static within such as bag and is not flowed from one portion to another portion. The flexible treatment chamber of several embodiments of the invention does not initially contain the fluid. The fluid is pumped through the treatment chamber 702 from the input tube 704 (supply conduit) to the output tube 706 (output conduit). As the fluid is flowed through the treatment chamber, the fluid expands the flexible treatment chamber and establishes a flow treatment volume. As the fluid is flowed through the treatment chamber, the fluid is treated with light. Using the proper flexible and light transmissive materials, the treatment chamber 702 is inexpensive to manufacture and is easily replaceable. For example, if such a treatment chamber were made of a rigid quartz material, such a treatment chamber would be more expensive to manufacture and would have to be cleaned after each use. Furthermore, it has been found that adhesives used to manufacture such a quartz treatment chamber react negatively with certain types of biological fluids and blood plasma derivatives. Advantageously, because the treatment chamber 702 is disposable, the treatment chamber 702 does not have to be cleaned, it is simply replaced after usage.

It is noted that depending on the desired flow rate and the type of fluid product to be pumped through the treatment chamber 702, the dimensions of the treatment chamber 702 may be altered. For example, the treatment chamber could be made longer or wider. The flow chamber 712 could be made wider or narrow, as well.

Referring next to FIG. 7B, a side view is shown of the treatment chamber 702 of FIG. 7A. As can be seen, the treatment chamber 702, the body portion 714, including the exterior edges 716, the flow chamber 712 and the boundary 718 are substantially flat, even with the presence of the preforms (see FIG. 7A) formed in the flow chamber 712. As shown at taper sections 722 and 724, the flow chamber 712 tapers outward to form the input port 705 and the output port 707, respectively. Also illustrated are input and output tubes 704 and 706 which couple to quick disconnects 708 and 710. As shown, fluid is not flowing through the flow chamber 712. Advantageously, the treatment chamber 712 provides a thin fluid flow path the width of the flow chamber 712. Furthermore, in this embodiment, the treatment chamber is designed to be a flexible flat treatment chamber.

Referring next to FIG. 7C, a schematic view of a transition from a circular flow profile to a substantially flat profile at the input and output of the treatment chamber of FIGS. 7A and 7B according to another embodiment of the invention. At the input port and the output port 705 and 707 of the treatment chamber of FIGS. 7A and 7B, the fluid flow has a generally circular cross sectional profile 726 (defined by the diameter d of the input and output tubes). However, when the treatment chamber is positioned between two plates, for example, light transmissive plates, the flow chamber 712 has a relatively flat cross sectional profile 728 with an adjustable thickness (depending upon the spacing of the two plates). Thus, according to this embodiment, the circular flow profile is to be transitioned or redistributed to a substantially flat flow profile. This is accomplished in the taper at taper section 722 (and 724). In preferred embodiments, it is desired that the transition take place such that the laminar fluid flow through the flow chamber 712 has substantially the same velocity across the width of the fluid flow. Thus, by carefully designing the taper section 722, the fluid flow being illuminated (e.g., within the treatment zone 730 portion of the flow chamber 712) has a substantially uniform, stream-lined velocity across its width.

Thus, the taper section 722 (and 724) is carefully configured to provide a smooth transition from the circular to the substantially flat profile. According to one embodiment, the length of the taper section 722 is approximately equal to 10 times the diameter of the circular fluid profile entering the taper section 722. Once the fluid flow exits the taper section 722, according to one embodiment, a distance of approximately 2 times the diameter of the circular fluid profile, is required to streamline the relative velocities of portions of the fluid flow in-line, such that when the fluid flow enters the treatment zone 730, the fluid flow will effectively be translated to a substantially laminar flow having substantially the same velocity across the width of the flow chamber 712, i.e., the fluid flow is a substantially uniform, streamlined velocity. A similar taper is formed at the taper section 724 at the output port of the treatment chamber to redistribute the laminar flow back to a circular flow, preferably having the same distance from the treatment zone 730 to the beginning of the taper section 724 and from the beginning of the taper section 724 to the output port 707.

Advantageously, by appropriately sizing the taper sections 722 and 724, dead spaces, stagnation and eddies are prevented from forming in the treatment zone 730 of the flow chamber 712, i.e., a substantially uniform fluid flow results. Thus, a smooth transition from the tube to the flow chamber 712 occurs at the input port 705. Also, the transition back to the substantially circular flow at the output port 707 is smooth in order to not disrupt the flow within the treatment zone 730. It is also noted that in some embodiments, the flow through the treatment chamber or treatment zone may be designed so as to not be uniform and even turbulent. It is further understood that a treatment chamber in accordance with several embodiments of the invention is not required to have a taper section as described above.

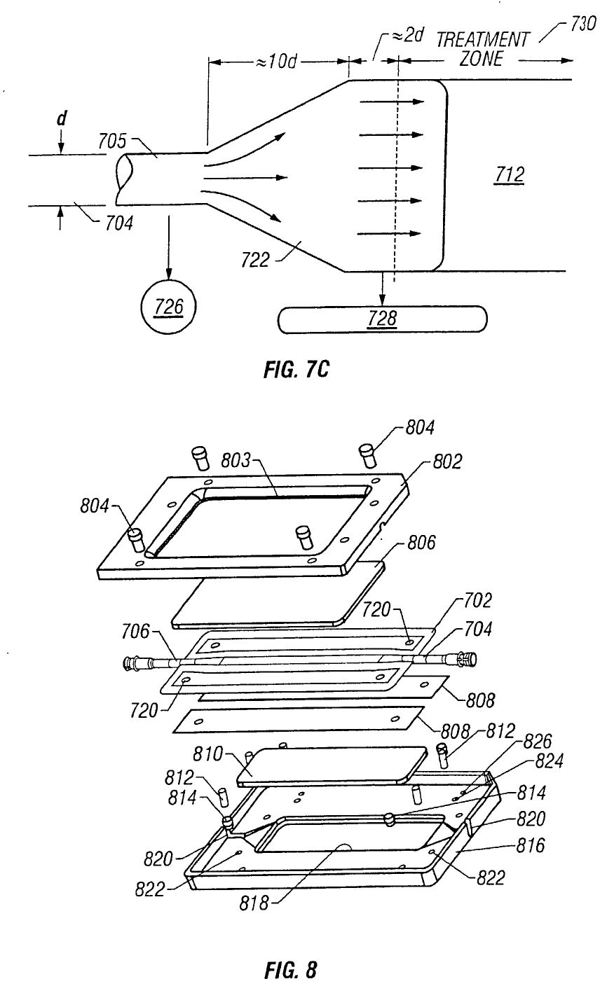

Referring next to FIG. 8, an exploded view is shown of one embodiment of the cartridge as shown in FIGS. 1-3 illustrating the treatment chamber of FIG. 7 positioned therein. Illustrated is a cartridge 800 including a cartridge top 802, cartridge top opening 803, screws 804, a first window 806 (also referred to as a first light transmissive window or plate or generically, a light transmissive support structure), the treatment chamber 702, opaque pieces 808, a second window 810 (also referred to as a second light transmissive window or generically as a plate portion or support structure portion), alignment pins 812 (referred to generically as alignment features), spacers 814, a cartridge bottom 816, alignment pin holes 822 (referred to generically as corresponding alignment features), spacer holes 824, threaded holes 826, a cartridge bottom opening 818 and slots 820. It is noted that the cartridge top 802 and the cartridge bottom 816 may be referred to generically as “parts” of a cartridge body.

The first window 806 is attached or adhered within the opening 803 of the cartridge top 802. The first window 806 is designed to be transmissive to at least a portion of the light treatment. The second window 810 is attached or adhered in position within the cartridge bottom opening 818 and is also transmissive to at least a portion of the light treatment. For example, the first window 806 and the second window 810 are transmissive to at least 1% of light having at least one wavelength within the range of 170 to 2600 nm. The first window 806 and the second window 810 are preferably made of quartz or similar material. The spacers 814 and the alignment pins 812 are attached to the cartridge bottom 816 within spacer holes 824 and alignment pin holes 822, respectively. Optionally, opaque pieces 808 are positioned on top of the cartridge bottom 816 such that they fit over the alignment pins 812 and block light from the sides so that the light entering through the second window 810 (to process monitors, such as a fiber probe or photodetector) is the light transmitted through the flow chamber. Next, the treatment chamber 702 is positioned over the opaque pieces 808 within the cartridge bottom 816. The alignment pins 812 extend through the holes 720 of the treatment chamber 702 to ensure alignment. Next, the cartridge top is positioned over the treatment chamber 702 and the screws 804 are threaded into the threaded holes 8826 of the cartridge bottom 816 to the desired tightness. Using the spacers 814 (e.g., 1-5 mm thick), a variable thickness between the first window 806 and the second window 810 can be achieved. Note that the input tube 704 and the output tube 706 fit within the respective slots 820 of the cartridge 800. It is noted that the second window 810 is not required to light transmissive. In embodiments where the second window 810 or plate portion is not light transmissive, the second window could be integrated into the cartridge bottom 816. It is preferably light transmissive to enable measurement of the light treatment that transmits through the fluid and to avoid reflections back into the treatment chamber. It is noted that in some embodiments, cooling plates or cooling components may be positioned against or within the cartridge structure in order to cool the treatment chamber within the cartridge in applications in which the treatment chamber is exposed to prolonged exposure or the temperature of the treatment chamber is otherwise is required to be controlled.

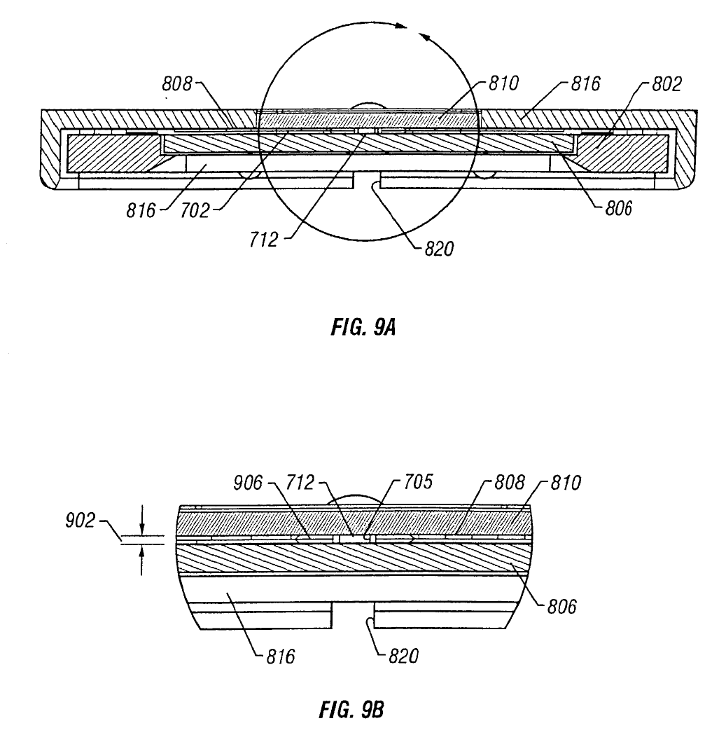

Referring next to FIGS. 9A and 9B, cross sectional views are shown of the cartridge of FIG. 8 containing the treatment chamber of FIGS. 7A-7B according to one embodiment of the invention. The view of FIG. 9A is a full cross sectional view across the width of the cartridge 800, while the view of FIG. 9B is an enlarged view of the portion of the view of FIG. 9A illustrating the flow chamber. As illustrated, the treatment chamber 702 is held between the first window 806 (or plate) and the second window 810 (or plate). As fluid flows through the flow chamber 712, the flow chamber expands or fills up to establish a flow geometry of the treatment zone, to create backpressure and to remove air bubbles. However, in this embodiment, since the flow chamber 712 is positioned between rigid plates, i.e., the first and second windows 806 and 808, the flow chamber 712 is forced to have a substantially uniform thickness 902 across the width of the flow chamber 712 and through the length of the flow chamber 712. As such, advantageously, the fluid flows through the flow chamber 712 substantially uniformly such that the light treatment penetrates all portions of the fluid to the same extent. In some embodiments, it is important to ensure that all portions of the fluid are treated equally, rather than some portions of the flow chamber being thicker than other portions, in the event such a flow chamber 712 were tubular. Also illustrated in the cross sectional view of FIG. 9B is the input port 705 (or alternatively, the output port 707). Line 906 represents the tapering from the input port 705 to the full width of the flow chamber 712.

It is noted that in alternate embodiments, the two support structures or plates, e.g., the first window 806 and the second window 810 may be curved or flat (as illustrated) and each may have a separate physical shape.

In some embodiments, the cartridge is not used, instead the treatment chamber 702 is mounted or positioned in front of a light source or lamp assembly containing a light source. In such alternative embodiments, the thickness of the flow chamber 712 may vary across the width of the flow chamber 712. Advantageously, by using the cartridge, the flow chamber 712 is sandwiched between two plates. Thus, this embodiment of a treatment chamber support structure restrains the flow chamber 712 such that it defines at least one dimensional boundary of the flow chamber 712, i.e., the top and bottom surfaces. At least one of these structures must be light transmissive, while the second plate may or may not be light transmissive. Thus, the first window 806 is light transmissive while the second window 810 is not required to be light transmissive. However, in preferred embodiments, the second window 810 is light transmissive to allow for optical detectors to view and measure the light penetrating through the treatment chamber and the fluid product and also to prevent reflected light from entering back into the treatment chamber.

It is noted that in some embodiments, the treatment chamber 702 does not have to be positioned within a cartridge for the flow chamber 712 to be substantially flattened. For example, the treatment chamber 702 (including the flow chamber 712) may be held or positioned against one or more support structures, e.g., positioned against one plate or sandwiched between two plates in order to sandwich the flow chamber 712 therebetween such that the flow chamber (or generically, the treatment zone) is restrained by the support structures (in this case, plates or windows). Thus, in these embodiments, the treatment chamber support structure defines one or more dimensional boundaries of the flow chamber 712. In other words, the pressure of the fluid flowing through the flow chamber 712 establishes a flow geometry of the flow chamber 712 within the confines of the treatment chamber support structure. At least one of these plates is light transmissive, preferably both plates. For example, one of the plates may be window 128. It is also noted that in alternate embodiments the treatment chamber support structure may be such that the thickness of the flow chamber is variable along its length, i.e., not necessarily a flat or plate-like structure.

Referring next to FIG. 10, a perspective view is shown of the cartridge of FIG. 8 as positioned within the cartridge registration plate of the fluid treatment system of FIGS. 1-3 according to one embodiment of the invention. As seen, the cartridge 800 containing the treatment chamber, is positioned within the cartridge registration plate 132 of the treatment area enclosure 104. As such, the cartridge 800 is registered within the cartridge registration plate 132. The cartridge 800 slides underneath the process monitor housing 138 until it is flush with edge 1002 of the cartridge registration plate. The cartridge lock clips 136 and the cartridge retaining clip 133 hold the cartridge 800 in place. Thus, the cartridge inserts into the cartridge registration plate 132. Furthermore, the cartridge is thick enough such that the input tube 704 and the output tube 706 extend from the slots 820 without bending.

Also illustrated are the process monitors 137 and 139 that measure the light, e.g., pulsed light. As can be seen process monitors 139 view light from the light source that transmits or passes through the cartridge 800 and the treatment chamber, while process monitors 137 view the light emitted directly from the light source having passed through the window 128 (i.e., light illuminating the treatment chamber or incident light). It is noted that these process monitors 137 and 139 are shown from the back. The process monitors 137 and 139 face toward the light source located on the opposite side of the window 128. In some embodiments, one or more of the process monitors 137 and 139 may be optical detectors, such as photodiodes or other photodetectors as known in the art, while in other embodiments, the one or more of the process monitors 139 and 139 may be fiber probes coupled to fiber optic cabling that extends from the process monitor housing to the electronics and control portion of an optical monitoring system. For example, in some embodiments, these fiber optic probes are inputs to a spectroradiometer as known in the art that measures a fluence at multiple wavelengths of the light treatment simultaneously. In one embodiment, the two process monitors 137 are fiber optic probes or collectors each coupled to a 2-channel spectroradiometer that measures the incident light upon the treatment chamber, while the two process monitors 139 are fiber optic probes or collectors each coupled to another 2-channel spectroradiometer that measures the light transmitting through the treatment chamber and the product being treated. Such embodiments and other variations are described further below. In other embodiments, one or more of the process monitors 137 and 139 may be pressure transducers or thermopiles, as are known in the art.

Referring next to FIG. 11, another embodiment of the fluid treatment system of FIGS. 1-3 is shown. Several of the components of the fluid treatment system of FIGS. 1-3 are the same as previously described. In this embodiment, the cartridge 134 is not used to contain the treatment chamber 702. The treatment chamber 702 (i.e., one embodiment of the treatment chamber 610 of FIG. 6) is simply positioned within the cartridge registration plate 132 (which may be generically referred to as a treatment chamber mounting device or treatment chamber support structure) and held in place with clips. Thus, as described above, the cartridge is not used in all embodiments; however, the cartridge is preferred since it restrains the flow chamber of the flexible, light transmissive treatment chamber 702 in order to define at least one dimensional boundary of the treatment chamber. In preferred embodiments, the cartridge provides for a substantially uniform thickness of the fluid flow along the length of the treatment chamber. Furthermore, it is noted that a support structure or plate (preferably light transmissive) may be positioned to restrain or sandwich the flow chamber of the treatment chamber 702 against the window 128 (e.g., using clips or adjustable screws with spacers) to provide a substantially flat laminar flow (or curved or turbulent flow, as desired) through the treatment chamber 702 without requiring that the treatment chamber be within a cartridge. In some embodiments, the clips press the flow chamber against the window 128; thus, the window 128 becomes the support structure that defines one dimensional boundary of the flow chamber of the treatment chamber 702. In embodiments where the treatment chamber is held between two plates or windows, the two plates or windows become a support structure that defines two dimensional boundaries of the flow chamber. Again, advantageously, the entire treatment chamber, as well as all of the components in the fluid flow path, are disposable upon completion of the fluid run.

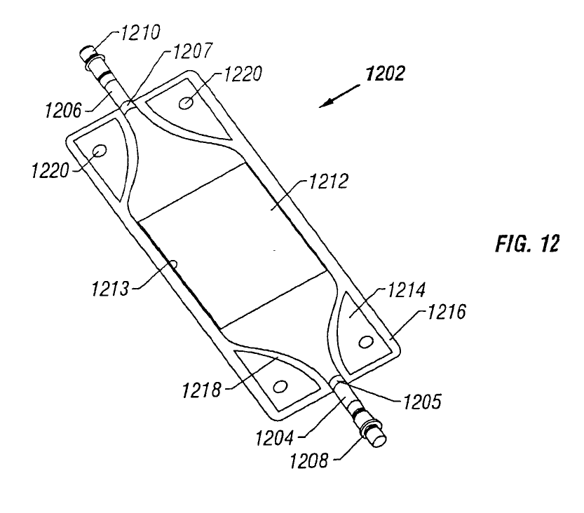

Referring next to FIG. 12, a perspective view is shown of a flat, disposable treatment chamber that may be used in the fluid treatment system of FIGS. 1-3 in accordance with another embodiment of the invention. Shown is the treatment chamber 1202 including an input tube 1204 coupled to an input port 1205, an output tube 1206 coupled to an output port 1207, each having a respective quick disconnect 1208 and 1210. The input tube 1204 and the output tube 1206 are round tubes coupled to the input and output ports 1205 and 1207. The input and output ports 1205 and 1207 taper into a flow chamber 1212 of the treatment chamber 1202. Similar to that shown in FIG. 7C, the taper section may be designed to smoothly transition the circular fluid flow to minimize dead spots or stagnation or to achieve a substantially flat laminar fluid flow. The flow chamber 1212 extends from the input port 1205 to the output port 1207. It is noted that the flow chamber 1212 may be generically referred to as a treatment zone or portion of a fluid flow path. The body portion 1214 of the treatment chamber 1202 is generally formed using multiple sheets of light transmissive material, such as described with reference to FIGS. 6 and 7A. These sheets are placed on top of each other and sealed together at the exterior edges 1216 and at the boundary 1218 to the flow chamber 1212. For example, the sheets of material are welded (e.g., radio frequency (RF) welded), or other wise bonded to each other to form the treatment chamber 1202. In some embodiments, a preform 1213 is formed in the sheets of material prior to being bonded or attached together. This preform helps that flow chamber to form as a chamber and to expand when fluid is flowed therethrough without creasing or bending along the bonded or attached portion. Thus, the treatment chamber 1202 is a generally flat and flexible structure.

The treatment chamber 1202 of FIG. 12 is similar to the treatment chamber 702 of FIGS. 7A and 7B; however, the width of the flow chamber 1212 is increased in comparison to the flow chamber 712 of FIGS. 7A and 7B. Advantageously, this allows for a greater flow rate to be obtained than with the treatment chamber 712. In one embodiment, a flow rate of 11 liters/min is obtained using the treatment chamber 1202 (in comparison to 1 liter/min with treatment chamber 702). Thus, the treatment chamber 1202 is another embodiment of a flexible, flat treatment chamber that is disposable. Additionally, holes 1220 (generically referred to as alignment features) are punched into the body portion 1214 to allow for alignment within a cartridge, such as the cartridge described above. When used with a cartridge, the light transmissive plates (windows) of the cartridge restrain the flow chamber 1212 to have a substantially flat profile across the width of the flow chamber 1212 and throughout the length of the flow chamber 1212. This provides for the uniform treatment of the fluid product through all portions of the flow chamber 1212.

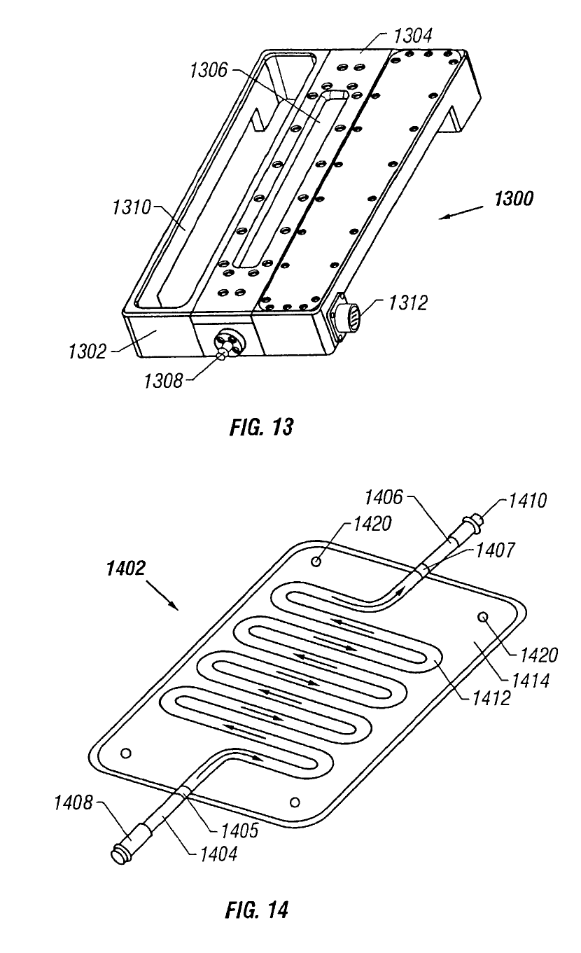

Referring next to FIG. 13, a perspective view is shown of a reusable, non-disposable treatment chamber according to another embodiment of the invention. The treatment chamber 1300 has a rigid body 1302 including a central back plate 1304 that contains a first window plate 1306. Opposite the central back plate 1304 and the first window plate 1306, is a central front plate (not shown) including a second window plate (not shown). A flow chamber (between the first and second window plates) is formed within the body portion 1302. The flow chamber may have a tubular cross section or a substantially flat cross section through the body 1302. An input port 1308 and an output port (not shown in this view) allow connection to the various flow tubes of the fluid flow path. Similar to the flexible, disposable treatment chambers of FIGS. 6-7B and 12, the reusable treatment chamber 1300 forms a flow chamber between the input port 1308 and the output port. Since the body portion 1302 is rigid, the thickness of the flow chamber can be controlled, i.e., the distance between the first and second window plates can be precisely controlled based upon the manufacturing specifications. In operation, fluid is flowed in through the input port 1308, through the flow chamber and out through the output port. As the fluid passes between the first and second window plates, the fluid is subjected to the light treatment, e.g., pulsed light treatment, to deactivate microorganisms within the fluid.

Also formed within the body portion 1302 is a handle portion 1310 to allow the operator to hold the treatment chamber 1300. It is noted that since portions of the treatment chamber 1300 are opaque, there may be a potential for slight shading to occur within portions of the flow chamber.

Additionally, in some embodiments, an electrical output 1312 is provided. Incorporated into the body portion are optional thermocouples and pressure transducers that will measure the temperature of the flow chamber and the pressure being exerted by the fluid therein, respectively. The electrical signals generated by these thermocouples and pressure transducers are output through the electrical output 1312. Thus, an electrical component adapted to mate with the electrical output 1312 transmits these signals to the system controller.

Since this treatment chamber is reusable, the treatment chamber should be cleaned and sterilized in between fluid runs. Disadvantageously, this may require disassembling the treatment chamber and cleaning it, for example, using an autoclave or other chemical flush.

Referring next to FIG. 14, a perspective view is shown of a flat, disposable treatment chamber that may be used in the fluid treatment system of FIGS. 1-3 in accordance with another embodiment of the invention. Shown is the treatment chamber 1402 including an input tube 1404 coupled to an input port 1405, an output tube 1406 coupled to an output port 1407, each having a respective quick disconnect 1408 and 1410. A radiator flow chamber 1412 extends from the input port 1405 to the output port 1407. The flow chamber 1412 may be generically referred to as a treatment zone or portion of the fluid flow path. The radiator treatment chamber 1402 is made from light transmissive materials, such as described with reference to FIGS. 6 and 7A. The radiator patterned flow chamber 1412 is welded into the body portion 1414.

The treatment chamber 1402 of FIG. 14 is similar to the treatment chambers of FIGS. 7A, 7B, and 12; however, the flow chamber 1412 is radiator shaped such that the fluid flow path winds back and forth across the width of the treatment chamber 1402 is it progresses along the length of the treatment chamber 1402 (as illustrated by the arrows in the flow path). Advantageously, such a flow path provides for more exposure of the fluid to the pulsed light, if a similar flash rate is used. This treatment chamber 1412 is another embodiment of a flexible, flat treatment chamber that is disposable. Additionally, holes 1420 (i.e., alignment features) are punched into the body portion 1414 to allow for alignment within a cartridge, such as the cartridge described above. When used with a cartridge, the plates of the cartridge press conform the flow chamber 1412 to have a substantially flat profile across the width of the flow chamber 1412 and throughout the length of the flow chamber 1412. This provides for the substantially uniform treatment of the fluid product through all portions of the flow chamber 1412. It is noted that this is just one variation of the potential for different flow paths within the treatment chamber 1402. Depending on the duration of exposure to the light, many other flow paths could be welded into a given treatment chamber. In another embodiment, the radiator design may simply comprise a radiator shaped tubing that is rigid and is held in position in front of the lamp assembly, e.g., positioned against window 128 of FIG. 1.

It is also noted that the treatment chamber 1402 may be positioned against one or more support structures or plates that define one or more dimensional boundaries of the flow chamber 1412. Also, in embodiments constructed of sheets of flexible material bonded together, preforms may be formed in the sheets along the edges of the flow chamber to allow the flow chamber to fill with fluids without creasing or bending along the bonded locations.

### Light Treatment Monitoring and Data Collection

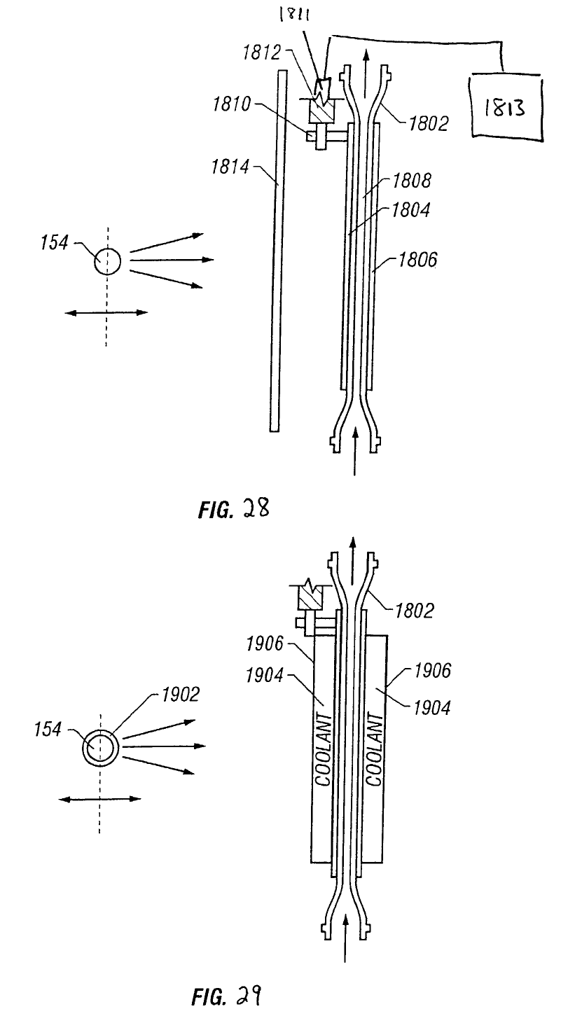

This section describes several methods and apparatus relating to the monitoring of and measurement of the light treatment, as well as the collection of data related to the light treatment and other system parameters in the use of treatment systems using light treatment for the treatment of products, e.g., the deactivation of microorganisms. Many of the measurements from the various monitoring methods described herein are also used by a controller or control system for analysis and feedback. Thus, the results of the various data monitoring techniques are input to an appropriate controller. Such controller methods and appratus are described further with reference to FIGS. 28-44.

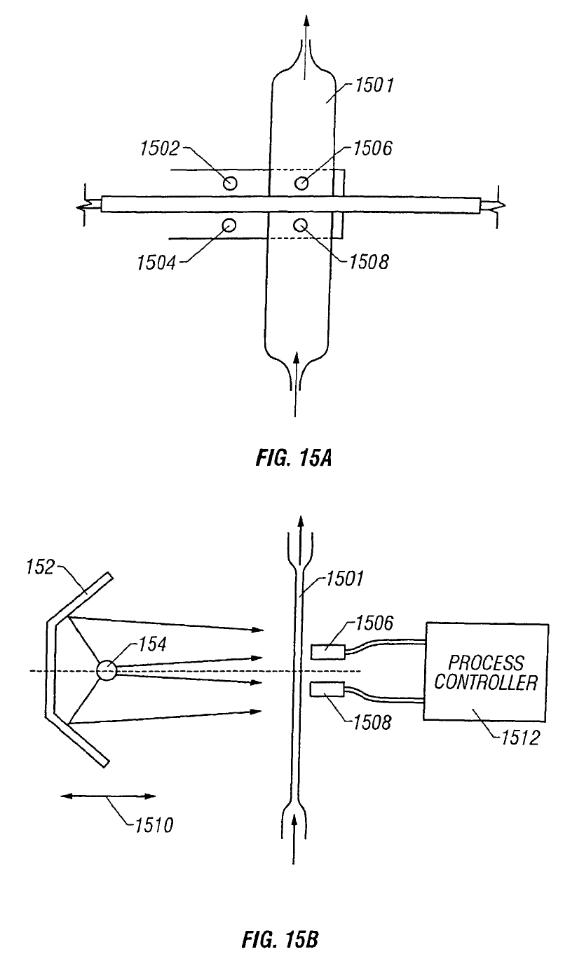

Referring next to FIG. 15A, a simplified front view is shown illustrating the relationship between the treatment chamber, the light source and the respective process monitors according to one embodiment of the invention. Concurrently referring to FIG. 15B, a simplified side view is shown of the treatment chamber, the light source and the respective process monitors. In FIG. 15A, the light source 154, e.g., flashlamp, is oriented to illuminate at least a portion of the treatment chamber 1501 (e.g., treatment chambers 610, 702, 1202, 1402). In other words, the light source 154 is positioned to illuminate a treatment zone of the fluid flow path of the system. Photodetectors 1502 and 1504 (i.e., one embodiment of the process monitors 137) are positioned to view the light emitted directly from the light source 154 that reaches the treatment chamber 1501. For example, in the fluid treatment system of FIGS. 1-3, photodetectors 1502 and 1504 (i.e., one embodiment of the process monitors 139) view the light transmitting through the window 128 of the cartridge registration plate 132. Photodetectors 1506 and 1508 are positioned to view the light emitted from the light source 154 and penetrating through the treatment chamber 1501 and its fluid contents. For example, in the fluid treatment system of FIGS. 1-3, photodetectors 1506 and 1508 view the light transmitting through the window 135 of the cartridge 134 and the registration plate window 128. This allows for measurements of the fluence or intensity and the spectral content of the light reaching the treatment chamber 1501 as well as the light penetrating through the fluid product.

Additionally, since the light emitted from the light source 154 includes wavelengths from about 180 nm to 2600 nm, the photodetectors 1502 and 1506 of this embodiment are ultraviolet photodetectors or photodiodes, e.g., they measure light having wavelengths between about 230 and 400 nm. Thus, photodetectors 1502 and 1506 provide an accurate characterization of the fluence and spectral content of the UV portion of the emitted light. For example, photodetectors 1502 and 1506 incorporate spectral filters that pass UV light. Furthermore, photodetectors 1504 and 1508 of this embodiment are full spectrum photodetectors are photodiodes that measure light having wavelengths between about 400 and 950 nm. For example, photodetectors 1504 and 1508 incorporate spectral filters that pass light between about 400 nm and 950 nm. Advantageously, the photodetector pairs behind the treatment chamber and to the side of the treatment chamber each include one UV photodetector and one full spectrum photodetector. It is noted that other photodetectors may be used depending on the wavelength range of the emitted light and system configuration. Thus, the photodetectors may be configured to measure light in any given range of wavelengths or of a desired single wavelength.

The photodetectors 1502 and 1504 are used, in one example, to verify the fluence selected by the operator prior to operation and the fluence of each flash during operation, as well as the spectral content of the light. For example, if the operator sets the fluence level to 0.3 J/cm², before the fluid run is initiated, the power to the light source 154 is set and the light source 154 is moved in the direction of arrow 1510 such that the distance between the light source 154 and the treatment chamber 1501 is set (e.g., using the linear slide servo drive 110). The light source 154, e.g., a flashlamp, is then flashed and the fluence is measured using photodetectors 1502 and 1504. If the fluence is not at the expected level, the distance between the light source 154 and the treatment chamber 1501 is incrementally adjusted based on the pre-learned adjustments and flashed again until the photodetectors verify the selected fluence. At this point, the product run is initiated. This is an important feature when the fluid product to be treated is a blood plasma derivative or other bioprocessing media, due to the sensitive nature of the fluid product. For example, exposure to light having a high fluence level may deactivate microorganisms, but may further result in an unacceptable amount of protein damage. In some instances, such bioprocessing fluid media may be extremely expensive and/or not replaceable, such that it is important that the fluence levels are accurately set by the fluid treatment system.

It is noted that each of the process monitors may measure one or both of the fluence level of the measured light and the spectral content of the measured light. It is also noted that in some embodiments, one or more of the process monitors 1502, 1504, 1506 and 1508 may comprise an optical detector such as a photodetector, a photodiode, a fiber optic probe coupled to an optical detector, a calorimeter, a joulemeter, a photomultiplier tube, a camera, and a CCD array. Furthermore, in some embodiments, the process monitors are fiber optic probes coupled to a spectroradiometer that is capable of measuring the fluence or intensity level of multiple wavelengths of the light treatment at one time. In other embodiments, the one or more of the process monitors 1502, 1504, 1506 and 1508 may comprise a thermodetector such as a thermocouple, a thermopile, a calorimeter, and a joulemeter.

A side view is illustrated in FIG. 15B. In this view the reflector 152 directs the light toward the treatment chamber 1501. Also seen are the UV photodetector 1506 and the full spectrum photodetector 1508. Furthermore, FIG. 15B illustrates a process controller 1512 that inputs the signals from the various process monitors and processes them to model the spectral content and/or the fluence level or intensity of the light treatment. This monitoring is used to adjust and verify the operating parameters of the fluid treatment system.

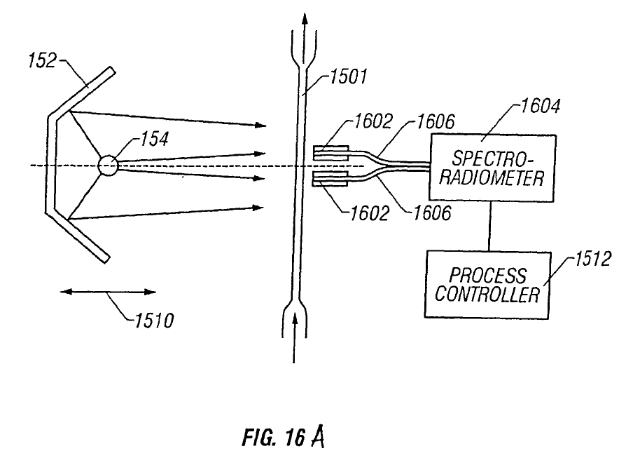

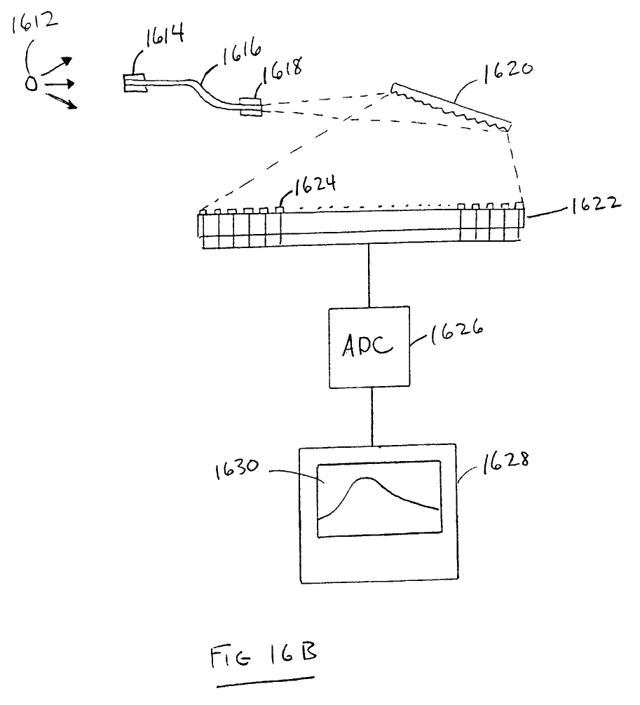

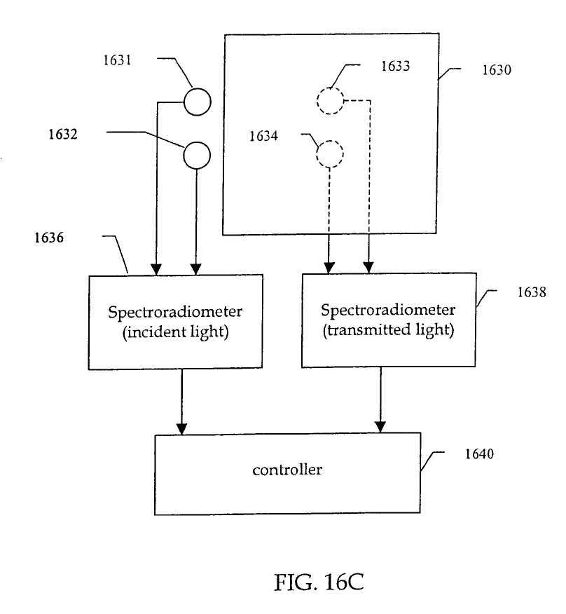

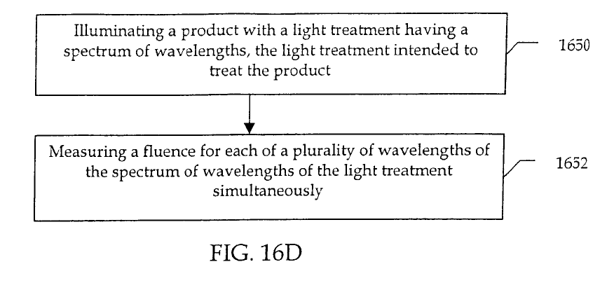

It is noted that in other embodiments, the photodetectors 1502, 1504, 1506 and 1508 may be replaced by fiber optic probes that are coupled to a spectroradiometer via fiber optic cables that measure separate fluence levels for multiple wavelengths of both the UV and full spectrum simultaneously for light transmitting through the treatment chamber and light emitted directly from the light source 154, as is described with reference to FIG. 16A. Alternatively, such fiber optic probes may be couple to a simple spectrometer that measures fluence in a binary sense that there is either fluence or not fluence at multiple wavelengths simultaneously; thus, the spectra of the measured light may be obtained without precise fluence or intensity level measurements of the light at each wavelength. As such, as used herein, a spectrometer is used to measure fluence at multiple discrete wavelengths of the spectrum of collected light, such fluence measurements may be quantified, for example, using a spectroradiometer at each of those discrete wavelengths. Thus, as used herein, the term “spectrometer”.

It should be noted that in preferred embodiments of the invention, reflective surfaces are not employed on the through side of the treatment chamber 1501. For example, referring briefly to FIG. 8, the window 810 is light transmissive. The window 810 could be made into a reflective surface that reflects light reaching through the treatment chamber back toward the treatment chamber. However, it has been found that this additional reflected light has an effect on the fluence levels as measured by a photodetector viewing light within the chamber, i.e., the fluence level appears slightly higher than is that truly emitted from the flashlamp 154. Due to the sensitive nature of some fluid products to be treated, it is more important to obtain a consistent and accurate measurement of the fluence of the emitted light, rather than maximize the fluence within the treatment chamber. Thus, in preferred embodiments, reflective surfaces are not employed on the through side of the treatment chamber 1501.

Referring next to FIG. 16A, a simplified side view is shown of a variation of the process monitoring system of FIGS. 15A and 15B according to another embodiment of the invention. According to this embodiment, rather than using discrete photodiode type photodetectors as the process monitors, fiber optic probes 1602 are provided in place of the photodetectors 1502, 1504, 1506 and 1508. Thus, the fiber optic probes 1602 are one embodiment of the process monitors 137 and 139. The light treatment, e.g., the output of each flash of a pulsed light treatment, is sampled directly illuminating the treatment chamber and transmitting through the treatment chamber 1501 via fiber optic probes 1602, which are coupled via fiber optic cables 1606 to a spectroradiometer 1604 (which may be generically referred to as a “spectrometer”) as is known in the art. The spectroradiometer 1604 takes the light collected at a single collection point and takes separate fluence measurements at multiple wavelengths across the spectrum of the light treatment simultaneously. The output of the spectroradiometer 1604 is analyzed in real time by the process controller 1512 to assure that each flash contains the proper distribution of wavelengths at the proper fluence levels or intensities, which is optimized depending on the specific microorganism or fluid product to be treated. It is noted that in embodiments using continuous wave light, the spectroradiometer is configured to process the light continuously.

The spectroradiometer 1604 is a multi-channel device including an analog to digital converter. In one embodiment, the fiber optic probes 1602 are cosine corrected irradiance probes (e.g., each probe has a teflon covering which acts as a diffuser), which are coupled to the analog to digital converter of the spectroradiometer 1604 via fiber optic cables 1606, e.g., 200, 300, or 400 μm or other diameter fiber optic cables. The spectroradiometer 1604 is integrated with software that measures the spectral intensity (fluence) of each flash from the light source 154. In one embodiment, similar to that described in FIGS. 15A and 15B, two probes measure UV light (225-400 nm) and the other two measure wavelengths from 400-950 nm, one of each type of probe measuring the light directly emitted from the light source 154 and one measuring the light transmitted through the treatment chamber 1501. Preferably, each probe 1602 is coupled to a separate spectroradiometer which is configured to measure the desired portion of the light. For example, a first probe is coupled via fiber optic cabling to a first spectroradiometer that is configured to separate and measure UV light, e.g., 225-400 nm. Similarly, a second probe is coupled via fiber optic cabling to a second spectroradiometer that is configured to separate and measure light, e.g., 400-950 nm. Alternatively, each probe is coupled to a spectroradiometer that is configured to measure the full spectrum including UV and IR. In another embodiment, two probes are coupled to a single two-channel spectroradiometer, one probe for measuring light between 225-400 nm and the other probe for measuring light between 400-950 nm, such that the two-channel spectroradiometer measures discrete fluences between 225-950 nm.

In operation, whether using photodetectors 1502, 1504, 1506, 1508 or fiber optic probes 1602, prior to flowing the fluid through the treatment chamber 1501, the light source intensity is checked by flashing the light source 154. The detection system including the process controller 1512 verifies the correct spectral content and fluence or intensity. In these embodiments, the fluence can be verified at particular wavelengths within the spectrum of the collected light, rather than a single measurement taken by a conventional photodetector that represents the fluence over the collected spectrum. In the event there are multiple spectroradiometers, each may be coupled to the same process controller 1512 for analysis. In other words, the process controller 1512 verifies that the fluence across the various wavelengths of the spectrum of light treatment, also referred to as the spectral signature of the light treatment. If the spectral signature is not correct, the process controller 1512 will adjust the distance of the light source 154 to the treatment chamber 1501 in order to vary the intensity or fluence over the spectral distribution prior to initiating the fluid run. Additionally, as is known, adjusting the charge voltage across the light source 154, e.g., a flashlamp, will change the spectral distribution. For example, higher charge voltages will drive the flashlamp plasma to higher temperatures and increase the UV to visible IR ratio to be delivered to the treatment chamber 1501. Thus, the use of the spectroradiometer 1604 and process controller 1512 will allow for the control and optimization of these process parameters.

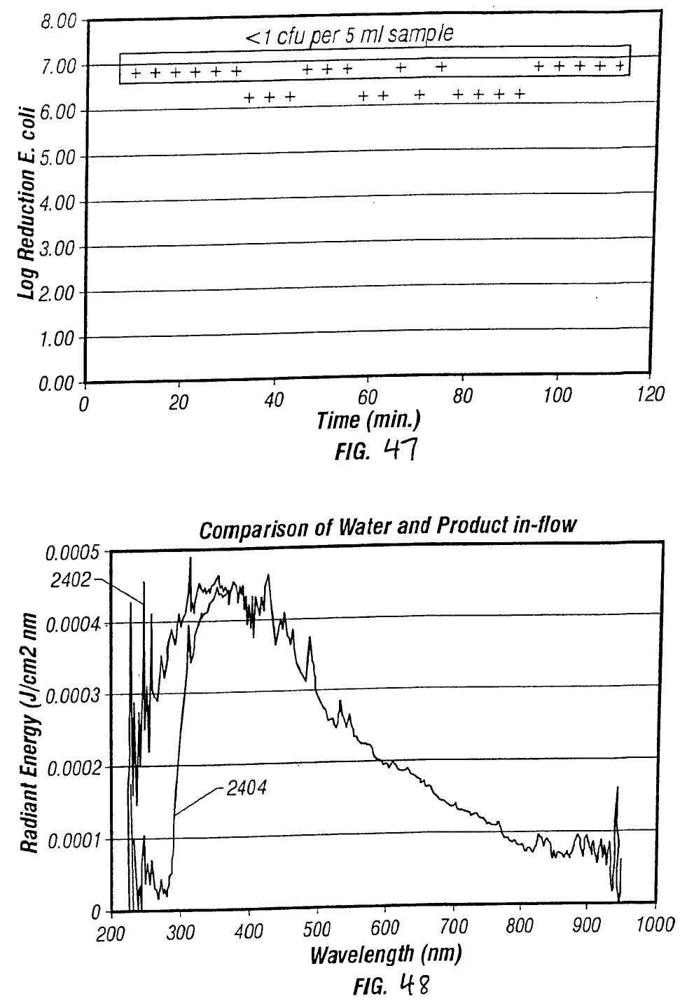

Furthermore, as the fluid product is flowed or pumped through the treatment chamber, light energy absorption is calculated and monitored at various wavelengths via the fiber optic probes 1602 that view the light penetrating through the treatment chamber 1501. For example, separate curves are generated for the spectral distribution of the light emitted directly from the light source (e.g., generated by one spectroradiometer) and for the light transmitting through the treatment chamber 1501 (e.g., generated by another spectroradiometer), an example of these curves is illustrated in FIG. 48. By integrating the two generated curves, two areas are obtained. By taking the difference between the two areas, the absorbed light energy is calculated at the various monitored wavelengths to generate an absorption curve, an example of which is illustrated in FIG. 17. This is an important metric to obtain since certain biological fluids, such as blood, blood plasma and blood plasma derivatives may incur excessive protein damage if the fluence level of the light is too high. As such, if too much energy is absorbed, there may be excessive protein damage. On the other hand, if too little energy is absorbed, microorganisms may not be deactivated to the desired levels.

Thus, due to the sensitivity of certain types of products being treated, such as bioprocessing fluids, blood plasma derivatives, etc., careful monitoring of the light treatment is needed. The use of the fiber optic probes 1602, fiber optic cable 1606 and the spectroradiometer 1604 enable accurate processing and modeling of the intensity (fluence) across the spectrum of the light treatment, while the control system of the fluid treatment system provides for adjustment of the spectral content and intensity of the light treatment in response to processing the light treatment.

Referring next to FIG. 16B, a diagram is shown illustrating one embodiment of the spectroradiometer (generically referred to as a spectrometer) of FIG. 16A to allow for the simultaneous measurement of fluence of a light treatment across multiple wavelengths of the light treatment spectrum. For example, the device of FIG. 16B may be used as the spectroradiometer of FIG. 16A or a light collection device in another light treatment system in which it is desired to measure the fluence of light across multiple wavelengths at the same time.

A light source 1612, such as a continuous wave or pulsed light source, produces light (i.e., a light treatment) having a spectrum of wavelengths, i.e., the light has multiple wavelengths present. For example, the light produced may be any range of light, such as 170-2600 nm, 200-1100 nm, 225-400 nm, 200-300 nm, 240-280 nm, etc. The light treatment is for the treatment of a product, e.g., in one embodiment, for the deactivation of microorganisms. At least a portion of the light is collected at collector 1614, e.g., a fiber optic probe, and is transported via fiber optic cable 1616 to an output 1618. It is noted that depending on the positioning of the collector 1614, a product to be treated with the light may be positioned in between the collector 1614 and the light source 1612, e.g., collector 1614 may be used as process monitors 1602 of FIG. 16A. Alternatively, the collector 1614 is positioned such that no product to be treated is located between the collector 1614 and the light source 1612, i.e., the collector 1614 collects light directly emitted from the light source or light that illuminates a product to be treated. It is noted that in one variation, the collector 1614 acts as a diffuser, i.e., it is a cosine corrected irradiance probe that allows for light incident at a variety of angles to enter the collector. Such probes are commercially available from Ocean Optics, Inc., of Dunedin, Fla., USA, Part No. CC3.

The collected light projects onto a grating 1620 which separates or splits the light into individual wavelength components which are projected onto an array 1622 of diodes 1624 or other optical detectors. For simplicity, not all of the individual diodes 1624 are illustrated. Furthermore, in this embodiment, it is noted that the light striking the right side of the array 1622 comprises light having shorter wavelengths while the light striking the left side of the array 1622 comprises light having longer wavelengths. An electrical signal is generated at each diode 1624, which is then coupled to an analog to digital converter 1626 (hereinafter referred to as ADC 1626). The digital output of the ADC 1626 is coupled to controller 1628 (such as the process controller 1512 of FIG. 16A or which may be implemented in the computer operating system of FIG. 41), which analyzes the data and in some embodiments, generates a plot 1630 of the fluence at each wavelength over the spectrum of wavelengths of the collected light, an example of which is illustrated in FIG. 48. Furthermore, in some embodiments, the curves for light illuminating the product and light transmitting through the product are compared to generate an absorption profile of the product to the light treatment (see FIG. 17, for example), which may be used to determine the total energy absorbed into a product for given exposure time (or pulse).

It is noted that the collector 1614, fiber optic cable 1616, output 1618, grating 1620, array 1622 of diodes 1624 and ADC 1626 are all common components of a spectroradiometer as is known in the art. Thus, one of skill in the art understands its operation. A spectroradiometer as known in the art is commercially available from Ocean Optics, Inc., of Dunedin, Fla., USA, Model No. S2000.

In preferred embodiments within a treatment system using a light treatment, a collector 1614 is positioned in front of the light source 1612 to measure the fluence of the emitted light and another collector 1614 is positioned on a throughside of a product (e.g., a fluid product) being treated with the light treatment such that the light transmitted through the product is measured. In embodiments measuring light transmitting through the product, the product to be treated may be a solid, liquid or gas that is at least partially transmissive to the light treatment. In preferred embodiments, the product is a fluid product (liquid or gas) that may be flowed through a treatment zone of a fluid flow path (or a treatment chamber) positioned to receive light energy from the light source 1612. Advantageously, the fluence of multiple wavelengths of both the direct light and the light transmitting through the product is measured simultaneously.

The use of a complex spectroradiometer to measure multiple wavelengths of light simultaneously in a light treatment device is a departure from the known art. Conventional light treatment systems, such as continuous wave UV light treatment systems, use a simple photodiode to measure the fluence of light at a single wavelength or a single fluence value for light having a range of wavelengths. Thus, these systems are monochromatic monitoring systems. Such systems typically measure the fluence at a given UV wavelength (or within a given range of UV wavelengths), since such wavelength is used for the light treatment. In contrast, a system employing a spectroradiometer is a polychromatic monitoring system that measures the fluence at many individual wavelengths within the spectrum of the light treatment. Furthermore, the light measured by the spectroradiometer is collected at a single collection point.

Referring briefly to FIG. 16C, an illustration of a treatment system is shown using multiple spectroradiometers for measuring incident and transmitted light according to one embodiment of the invention. A product 1630 to be treated is illuminated with a light treatment. Collectors 1631 and 1632 (e.g., fiber optic probes) collect the incident light illuminating the product 1630, while collectors 1633 and 1634 (e.g., fiber optic probes) collect light transmitting through the product 1630. It is noted that the product 1630 is transmissive to at least a portion of the light treatment and the product 1630 may be contained within a treatment chamber, for example, the product is a fluid product flowed through a treatment chamber. In one embodiment, collectors 1631 and 1633 collect UV light (e.g., 200-400 nm), while collectors 1632 and 1634 collect light beyond UV (e.g., 400-1000 nm). Collectors 1631 and 1632 are coupled (e.g., via fiber optic cables) to spectroradiometer 1636 while collectors 1633 and 1634 are coupled (e.g., via fiber optic cables) to spectroradiometer 1638. Each spectroradiometer 1636 and 1638 is a two-channel spectroradiometer such that one channel is for the light from the UV collector and the other channel is for the light from the other collector (400-1000 nm). As such, spectroradiometer 1636 generates discrete fluence measurements of light incident on the product for multiple wavelengths within the range of 200 nm to 1000 nm, while spectroradiometer 1638 generates discrete fluence measurements of light transmitting through the product 1630 for multiple wavelengths within the range of 200 nm to 1000 nm. The system controller 1640 uses these measurements for a variety of purposes, for example, in order to generate an absorption profile and absorption energy, for example, as described herein.

It is noted that there may be only one collector for the incident light and one collector for the transmitted light depending on the embodiment. For example, only collector 1631 is used to collect incident light within the desired spectrum, e.g., 200-1000 nm, 200-400 nm, etc., while only collector 1633 is used to collect transmitted light within the desired spectrum. As such, the spectroradiometers 1636 and 1638 may be single channel or multiple channel devices. Furthermore, the spectroradiometers 1636 and 1638 may be simple spectrometers that measure the existence of light at multiple wavelengths within the desired spectrum. Thus, these spectrometers measure fluence at multiple wavelengths in that there is either fluence or not fluence at the given wavelengths. Such embodiments may be employed where it is desired to know the spectrum of the incident and transmitted light, without requiring the fluence levels. Although there may be different configurations, it is noted that a separate spectrometer device is used for the incident light and the transmitted light.

Referring next to FIG. 16D, a flowchart is shown of the steps performed in accordance with one embodiment of the invention. In one embodiment, the steps of FIG. 16D may be performed by the apparatus of FIGS. 16A-16C in use within a treatment system using light for the deactivation of microorganisms, such as fluid treatment system 100 of FIG. 1; however, these steps may be performed using other structure. Initially, a product is illuminated with a light treatment, the light treatment having a spectrum of wavelengths, the light treatment intended to treat the product (Step 1650). It is noted that the product may be treated as described herein. In some embodiments, the product is contained within a treatment chamber or treatment zone of the treatment system, the light source being external to the treatment chamber. The product may be a fluid product or may be any type of product to be treated, for example, a gas, a liquid, or a solid. Additionally, the light treatment may be a pulsed light treatment including at least one pulse of light. Alternatively, the light treatment may be a continuous wave light treatment. It is noted that when the light treatment is for deactivating microorganisms, the nature of the deactivation depends on the product. For example, the deactivation of microorganisms may be within a gas or liquid product, but may be on the surface of a solid product or within a solid product if the solid product is transmissive to the light treatment.

Next, a fluence is measured for the light treatment for each of a plurality of wavelengths of the spectrum of wavelengths simultaneously (Step 1652). In several embodiments, a spectrometer is used to measure the fluence. For example, a spectroradiometer is used to measure a fluence level for each of the plurality of wavelengths. In another example, a simple spectrometer is used which measures fluence (without measuring the level of fluence) for each of the plurality of wavelengths. That is, the spectrometer simply measures the existence of fluence at each of the plurality of wavelengths or measures the spectral content of the light treatment at the plurality of wavelengths. In one embodiment, the apparatus of FIG. 16B is used such that at least a portion of the light treatment is collected at a collector and input to a spectroradiometer. The spectroradiometer then separates the spectrum of wavelengths into individual wavelengths which are directly to individual optical detectors.

This method is believed to be a departure from the known art in light treatment monitoring since the fluence at more than one wavelength of the light treatment is measured for different wavelengths at the same time from a single collection point. This is particularly important in pulsed light applications since the duration of an individual pulse of light may be extremely short. Thus, advantageously, in pulsed light treatment systems, the fluence is measured for multiple wavelengths of the spectrum of the pulsed light treatment for each flash of the light treatment.

In some embodiments, the fluence is measured for a portion of the light treatment illuminating the product and/or the fluence is measured for a portion of the light treatment transmitting through the product. In some embodiments, there is a separate collection point for the light illuminating the product and the light transmitting through the product. Thus, each collection point leads to one or more spectrometers, and each collection point yields discrete measurements of fluence at multiple wavelengths.

Referring next to FIG. 17, an absorption profile is illustrated in accordance with one embodiment of the invention. In systems using light having a spectrum of wavelengths, it is helpful to determine what is the fluence level across the spectrum of wavelengths of the light treatment. For example, by positioning one or more collectors (optical detectors) to measure light directly emitted by the light source and another one or more collectors to measure light transmitting through a product being treated (for example, see FIG. 16C), the difference between the two generated curves (e.g., plot 1630) can be used to determine the amount of light absorbed by the product across the wavelength range of the spectrum. This allows for a full spectrum absorption curve to be generated in real time. A controller or processor may be configured to generate a suitable absorption profile by those skilled in the art.

FIG. 17 is an example of such absorption profiles 1720, 1722 (also referred to as an absorption curve or optical signature of the product) at 5 flashes and 10 flashes, respectively, of a pulsed light source. It is noted that the more exposure to the light energy, the greater than absorption. The total absorbed energy per flash may be determined or calculated as an integration of the fluence at each wavelength over the spectrum of the light treatment. This total absorbed energy per flash may be stored and used as a metric to adjust and control the light treatment, e.g., to signal that treatment may be complete or that overtreatment will occur with further exposure. If the light treatment is a continuous treatment, this treatment may be segmented into intervals of time that are analyzed, i.e., a separate absorption profile may be generated at given intervals of time and analyzed.

Furthermore, although absorption profiles 1720, 1722 are illustrated generally within the UV range, e.g., 250 to 400 nm, the absorption profiles may be analyzed over any range of wavelengths of interest within the spectrum of the light treatment.

The absorption profile of a given product tells a great deal about the material being treated, such as, what types of molecules are present, types of atomic bonds are present in the material, the colors of the material, as well as the way different wavelengths of light energy interact with the material. In some embodiments, this absorption profile is monitored over time. For example, with some products, the absorption profile changes as the product is exposed to more light energy. In one embodiment using a pulsed light source and certain types of fluid products, as a given portion of a fluid product is exposed to further flashes of light, the wavelengths absorbed shift. For example, after 4 flashes, more light is absorbed at one wavelength and less at another wavelength in comparison to after 2 flashes. With some products that are treated, such shifts in the absorption profile indicate when a product is overtreated. Particularly with sensitive biological fluid products, too much light treatment damages the product itself. For example, in blood products, such as BSA, the absorption of wavelengths around 320 nm increases as proteins within the product are damaged. With careful monitoring of the absorption profile by being able to simultaneously measure the fluence at each wavelength across the spectrum of light and generate real time absorption profiles, overtreatment of a product may be avoided.

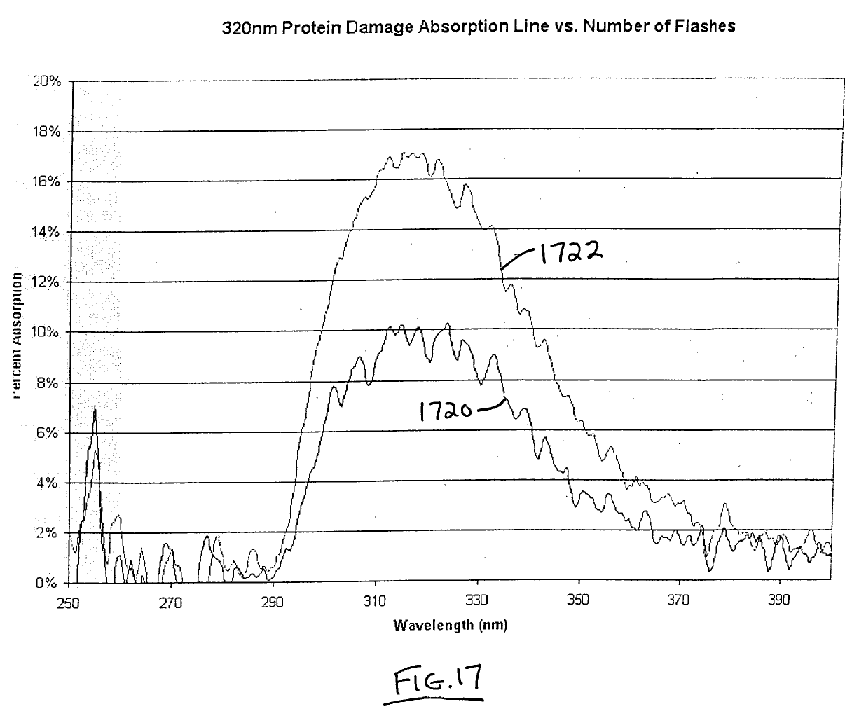

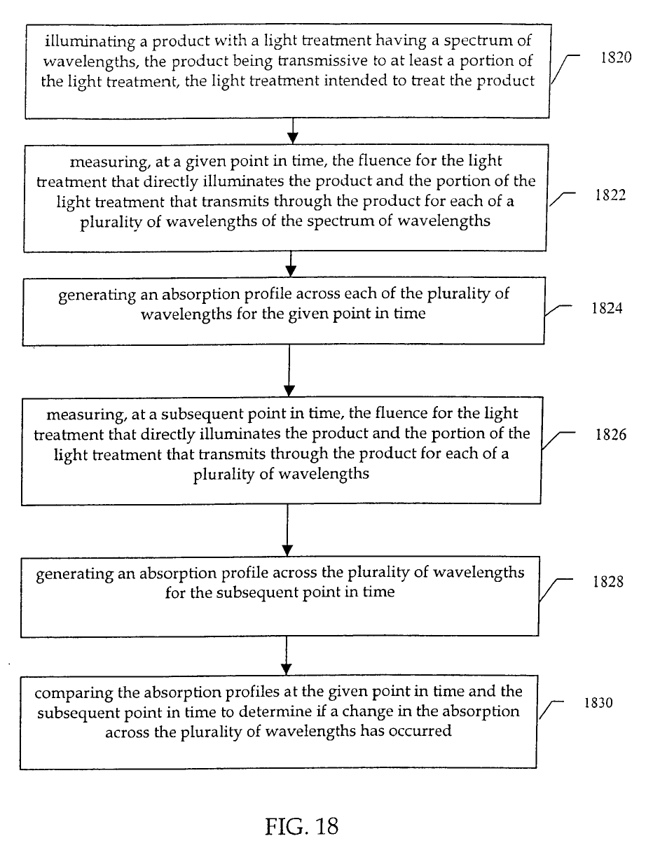

Referring next to FIG. 18, a flowchart is shown of the steps performed in another embodiment of the invention. In one embodiment, the steps of FIG. 18 may be performed by the apparatus of FIGS. 16A-16C in use within a treatment system using light for the deactivation of microorganisms, such as fluid treatment system 100 of FIG. 1. However, it should be appreciated that these steps may be performed by other light treatment systems having a variety of configurations for a variety of purposes. Initially, a product is illuminated with a light treatment having a spectrum of wavelengths, the product being transmissive to at least a portion of the light treatment, the light treatment intended to treat the product (Step 1820). The product may be a fluid product or may be any type of product to be treated, for example, a gas, a liquid, or a solid; however, should be transmissive to at least a portion of the light treatment, e.g., transmissive to at least 1% of light having at least one wavelength within a range of between about 170 and 2600 nm. The product may also be contained within a treatment chamber. It is noted that preferably, the product is flowed through a transmissive treatment chamber that is positioned to receive the light treatment. Additionally, the light treatment may be a pulsed light treatment including at least one pulse of light as described herein. Alternatively, the light treatment may be a continuous wave light treatment.

Next, at a given point in time, the fluence is measured for the light treatment that directly illuminates the product (is incident on the product) and the portion of the light treatment that transmits through the product for each of a plurality of wavelengths of the spectrum of wavelengths (Step 1822). In one embodiment, the apparatus of FIG. 16B is used such that at least a portion of the light treatment is collected at a collector positioned to receive light emitted directly from the light source and collected from a collector positioned to receive light transmitted through the product. The collectors input the light to one or more spectrometers, e.g., one or more spectroradiometers.

Next, an absorption profile across each of the plurality of wavelengths is generated for the given point in time (Step 1824). In one embodiment, the absorption profile is the difference between the fluence directly illuminating the product and the fluence transmitting through the product for each of the plurality of wavelengths of the spectrum. It is noted that in one embodiment, the controller 1628 (or controller 1640) performs Step 1824; however, such determination may be incorporated into the spectrometer or other processor or analysis system, e.g., within the computer operating system/user interface of FIG. 41.

In pulsed light treatments, absorption profiles may be generated on a per pulse basis and stored. Likewise, in continuous wave light treatments, the absorption profiles may be generated at preselected intervals and stored.

It is believed that the generation of such an absorption profile is a departure from the known art in treatment devices using light since the absorption is determined at multiple wavelengths within the spectrum of wavelengths, as opposed to a single wavelength or absorption single fluence level covering a range of wavelengths. Additionally, the absorption profile may be generated at a later point in time or, in preferred embodiments, the absorption profile is generated in real time as measured. Real-time determinations provide the treatment system with a tool to analyze and control the light treatment process as treatment occurs.

In some embodiments, the total energy absorbed into the product is determined, for example, by integrating the fluence measurements for each wavelength across the spectrum of the light treatment. The total energy absorbed may be made on a per pulse basis, or per time interval basis, and used by an appropriate controller to indicate the progress of treatment.

Next, at a subsequent point in time, the fluence is measured for the light treatment that directly illuminates the product and the portion of the light treatment that transmits through the product for each of a plurality of wavelengths of the spectrum of wavelengths (Step 1826) and an absorption profile across the plurality of wavelengths is generated for the subsequent point in time (Step 1828).

Depending on the embodiment, the given and subsequent points in time are variously defined. For example, in a pulsed light treatment system, the given and subsequent points in time coincide with a first pulse of light and a subsequent pulse of light. In a continuous wave light system, the given and subsequent points in time are simply at given intervals of time, e.g., every 0.5 seconds or other suitable interval.

The absorption profiles at the given point in time and the subsequent point in time are both compared to determine if a change in the absorption across the plurality of wavelengths has occurred (Step 1830). This comparison may be performed by the controller 1628 or 1640 or other processor or analysis system. As discussed above, changes or shifts in the absorption profile of a product can provide information about the proper treatment of a given product and at what point in time the product may be overtreated. Thus, according to several embodiments, the absorption profile is analyzed as a trigger that light treatment is at the appropriate level or that an operating condition has been met. For example, in one embodiment, operating conditions are a pass condition and a fail condition. In one embodiment, a pass condition indicates that the product is safe for further treatment, while a fail condition indicates that the product has been or will be overtreated.

In accordance with many embodiments, after the absorption profile is generated in Step 1824, the absorption profile is compared to a known valid absorption profile that has been previously recorded for the given product at the same system and treatment settings. This comparison may be performed by the controller 1628 or other processor or analysis system. In this comparison, it can be determined whether the measured absorption profile correlates to the known valid profile. Thus, it can be determined if there is a deviation from the known valid absorption profile and at what wavelengths there is a deviation. This information may be helpful in determining a fault in the system or light treatment parameters and may trigger the system controller to make the appropriate adjustments.

Furthermore, in accordance with many embodiments, after the absorption profile is generated in Step 1824, the absorption profile is also analyzed to identify an absorption peak at one of the wavelengths of the spectrum of the light treatment. This analysis may be performed by the controller 1628 or 1640 or other processor or analysis system. It may also be determined that there is more than one absorption peak. For example, in the absorption profile 1722 of FIG. 17, there is an absorption peak at about 315 nm and a smaller peak at about 255 nm. These absorption peaks can also tell a great deal about the product being treated. The identification of specific absorption peaks for a given product may have many uses. For example, absorption peaks may be stored for later analysis and comparison. Another use is to track changes in the absorption peak with additional exposure. For example, in some products, the absorption peak shifts after a certain level of exposure and may be used again to determine if the product is being overtreated and/or set pass/fail conditions of the treatment.

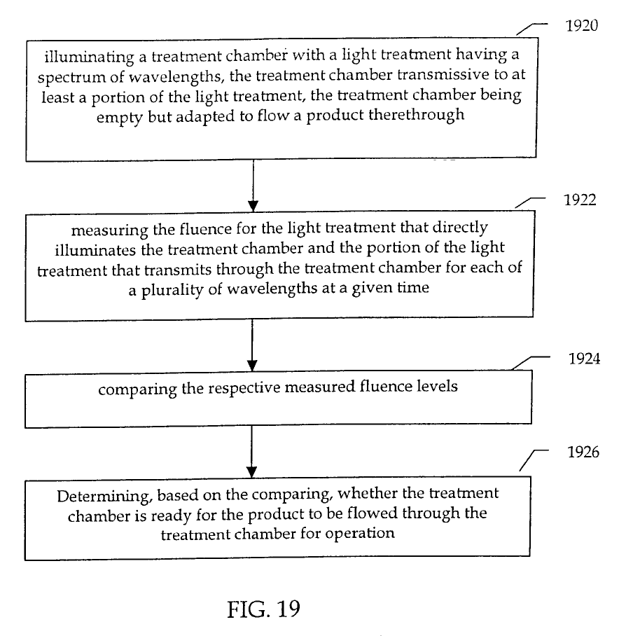

Referring next to FIG. 19, a flowchart is shown that illustrates the steps performed according to another embodiment of the invention. In one embodiment, the steps of FIG. 19 may be performed by the apparatus of FIGS. 16A-16C in use within a treatment system using light for treating products as described herein, such as fluid treatment system 100 of FIG. 1. However, it should be understood that these steps may be performed by other light treatment systems having a variety of configurations for treating a variety of products for a variety of uses.

Initially, a treatment chamber is illuminated with a light treatment having a spectrum of wavelengths, the treatment chamber transmissive to at least a portion of the light treatment, the treatment chamber being empty but adapted to flow a product therethrough that is to be treated with the light treatment (Step 1920). The treatment chamber may be empty since a given treatment has not yet started, has finished, or has been temporarily stopped. The product to be treated may be a fluid product or other product, for example, a gas, a liquid, or a solid. Additionally, the light treatment may be a pulsed light treatment including at least one pulse of light or a continuous wave light treatment as described herein.

Next, as described above, the fluence is measured for the light treatment that directly illuminates the treatment chamber and the portion of the light treatment that transmits through the treatment chamber for each of the plurality of wavelengths at a given time (Step 1922). For example, in one embodiment, a spectroradiometer is used to measure the measure level at each of the plurality of wavelengths. Next, respective measured fluence levels are compared for each of the plurality of wavelengths (Step 1924), and in some embodiments, an absorption profile for the plurality of wavelengths is generated.

Then, it is determined, based upon the comparing step, whether the treatment chamber is ready for the product to be flowed through the treatment chamber for operation (Step 1926). This may be determined when optical absorption profile of the treatment chamber is within an acceptable operating range defined by the user and/or system. This determination may be made by the system controller or other controller, e.g., process controller 1512, 1628, 1640. This process is used to determine, for example, the cleanliness of the treatment chamber and system components. For example, in use, the treatment chamber may accumulate deposits of materials that may affect the transmission of light therethrough. Thus, this method provides a technique to optically determine using optical measurements whether the system components, such as the treatment chamber or other transmissive structures are clean enough for the operation of the system, or have not been excessively fouled during treatment. For example, in the treatment system 100 of FIGS. 1-4, the light source is activated and measured prior to flowing the buffer fluid within syringe 120 or the fluid product within syringe 118.

These results may also indicate whether the transmissive components, such as the treatment chamber are properly aligned or installed. For example, if the treatment chamber is not aligned properly, reflections may reduce the light treatment transmission. Alternatively, if the window or other light transmissive portion of the treatment chamber is not in the proper location, portions of the light treatment may be blocked from illuminating and transmitting through the product.

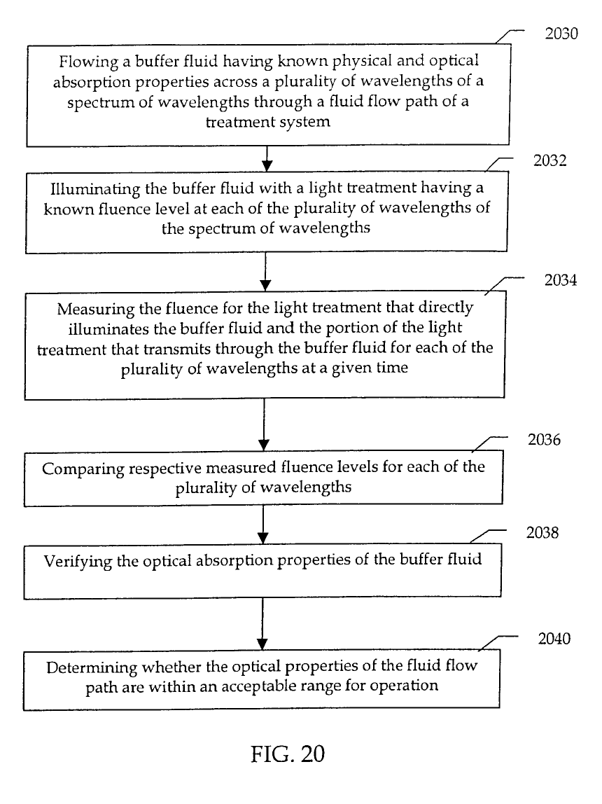

Referring next to FIG. 20, a flowchart is shown that illustrates the steps performed according to another embodiment of the invention. In one embodiment, the steps of FIG. 20 may be performed by the apparatus of FIGS. 16A-16C, or other apparatus in use within a treatment system using light for treating as described herein, such as fluid treatment system 100 of FIG. 1. However, it should be understood that these steps may be performed by other light treatment systems having a variety of configurations for treating a variety of products.

Initially, a buffer fluid is flowed through a fluid flow path of a treatment system (e.g., a treatment chamber), the buffer fluid having known physical and optical absorption properties across a plurality of wavelengths of a spectrum of wavelengths (Step 2030). The buffer fluid may be any fluid, such as those described herein. It is noted that the buffer fluid may also be referred to in this context as a “test fluid” that is used to test the treatment system prior to treating a fluid product. As such, the buffer fluid or test fluid flowed may be the same fluid as or a different fluid than a buffer fluid that is flowed to transition the fluid product being treated or mixed in with the fluid product at a given concentration or may be a separate fluid.

Next, the buffer fluid is illuminated with a light treatment having a known fluence level at each of the plurality of wavelengths of a spectrum of wavelengths (Step 2032). The light treatment is for the treatment of fluid products as described herein, for example, for the deactivation of microorganisms.

Next, as described herein, the fluence is measured for the light treatment that directly illuminates the buffer fluid and the portion of the light treatment that transmits through the buffer fluid for each of the plurality of wavelengths at a given time (Step 2034). Next, respective measured fluence levels are compared for each of the plurality of wavelengths (Step 2036), and in some embodiments, an absorption profile across the plurality of wavelengths is generated.

Next, the optical absorption properties of the buffer fluid are verified (Step 2038), e.g., by comparing the measured fluence levels at the plurality of wavelengths to the known optical absorption properties of the buffer fluid at the plurality of wavelengths, which have been previously stored.

Then, it is determined, based upon the comparing step, whether the optical properties of the fluid flow path at the plurality of wavelengths are within an acceptable range for operation (Step 2040). This determination may be made by the system controller or other controller, e.g., process controller 1512, 1628, 1640. This process is used to determine, for example, the cleanliness of the fluid flow path or treatment chamber and system components while in use with a buffer fluid rather than with the product to be treated or with a dry system, such as described with reference to FIG. 19. This method is particularly useful to determine the interaction of all of the system components, such as those governing flash rate, flow rate, fluence, treatment chamber geometry, treatment chamber cleanliness, while the system is in operation. This method represents a departure from the known art in light treatment systems.

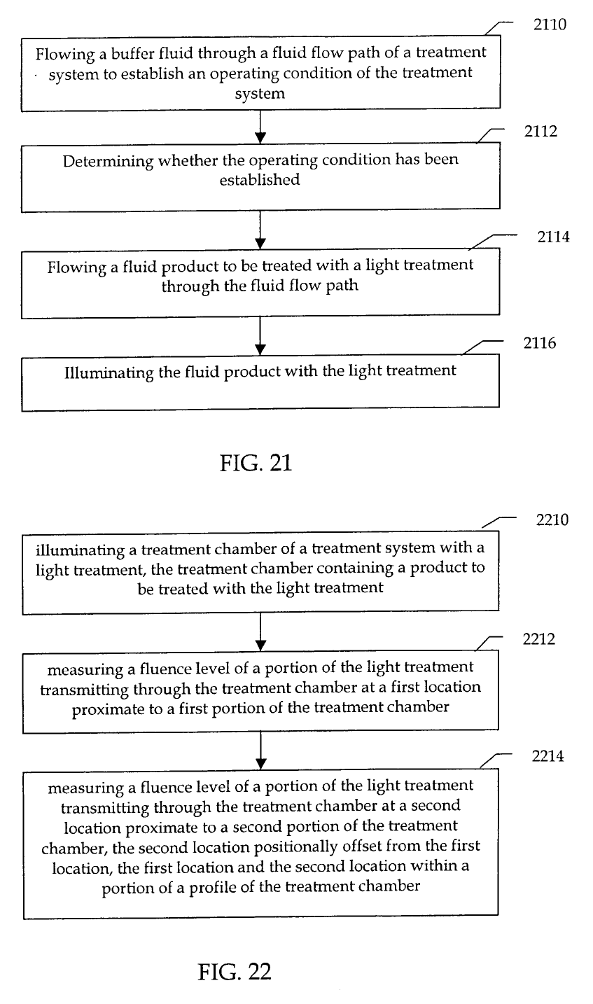

Referring next to FIG. 21, a flowchart is shown that illustrates the steps performed according to another embodiment of the invention. In one embodiment, the steps of FIG. 21 may be performed by the apparatus of FIGS. 16A-16C or other apparatus in use within a treatment system using light for treating products as described herein, such as fluid treatment system 100 of FIG. 1. However, it should be understood that these steps may be performed by other light treatment systems having a variety of configurations for treating a variety of products. Initially, a buffer fluid is flowed through a fluid flow path of a treatment system to establish an operating condition of the treatment system (Step 2110). The buffer fluid may be any fluid, such as those described herein. It is again noted that the buffer fluid may also be referred to in this context as a “test fluid” that is used to test the treatment system prior to treating a fluid product. As such, the buffer fluid or test fluid flowed may be the same fluid as or a different fluid than a buffer fluid that is flowed to transition to the fluid product being treated or mixed in with the fluid product at a given concentration. The operating condition is a condition that should be established in order for the light treatment process of the treatment system to work properly.

For example, in one embodiment, the operating condition is a treatment geometry that needs to be established within the fluid flow path, particularly, within the treatment chamber or treatment zone portion of the fluid flow path. As described earlier, in some embodiments having a flexible treatment chamber or flexible fluid flow path, the path must be pumped with a fluid that expands the flexible treatment chamber and forms and defines a treatment zone or treatment geometry for fluid products to flow therethrough and be illuminated with a light treatment. Thus, one operating condition is establishing a treatment geometry.

In another embodiment, the operating condition is that the fluid flow through the fluid flow path, or at least through the treatment chamber or treatment zone portion of the fluid flow path should be at the specified flow rate. This is particularly important in treatment systems using pulsed light sources since the flow rate is coordinated with the flash rate of the pulsed light source(s). This is also important in systems treating sensitive biological fluid products since care should be taken in order to ensure the desired minimum treatment, but to avoid overtreatment of the fluid product. Thus, in another embodiment, the flow rate is an operational condition that should be met.

Next, it is determined whether the operating condition has been established (Step 2112). For example, in the event the operating condition is that a treatment geometry is to be established, the system takes measurements of the pressure of the fluid flow, for example, at the entrance and exit of the treatment chamber or treatment zone portion of the fluid flow path. Once the pressure readings reach a preselected value or are within an acceptable range, such as determined by the appropriate controller, this indicates that the flexible treatment chamber has been appropriately inflated to establish the treatment geometry. It is noted that in some embodiments, the treatment chamber is forced outward into a support structure that defines one or more dimensional boundaries of the treatment geometry. By way of example, in the system of FIG. 6, pressure transducers 632 and 636 measure the pressure at the entrance and exit of the treatment chamber 610. The outputs of these pressure transducers are output to a control system that compares the measured values to stored values to determine if the condition has been met.

Alternatively, if the operating condition is that a specified flow rate is to be established, then flow rate sensors may be employed at the entrance and exit of the treatment chamber or treatment zone of the fluid flow path. Similarly, the outputs of the sensors are input to a control system that determines if the measured values are at preselected values or within a preselected range of values, indicating that the flow rate has been established.

Next, a fluid product to be treated with a light treatment is flowed through the fluid flow path (Step 2114). Then, the fluid product is illuminated with the light treatment (Step 2116). Advantageously, the operating condition has already been established through the use of the buffer fluid so that the treatment of the fluid product can be maximized, e.g., no fluid product is wasted, undertreated, overtreated, etc.

It is noted that the methods described herein, e.g., of FIGS. 16D, 18, 19, 20, and 21, may be performed by systems and structures described herein or in other treatment systems using light for the treatment of products, such as for the deactivation of microorganisms, such as viruses, bacteria, pathogens, etc.. For example, the light treatment may be any pulsed light treatment or continuous wave light treatment described herein or otherwise.

Referring next to FIG. 22, a flowchart is illustrated that lists the steps performed according to one embodiment of the invention in which fluence measurements are taken of light transmitting through a treatment chamber at multiple locations across the profile of the treatment chamber. The Steps listed in FIG. 22 provide the basis for the methods and apparatus provided below in FIGS. 23-25.

Initially, while referring to FIG. 22, concurrent reference will also be made to FIG. 23. FIG. 23 illustrates a simplified perspective view of a detector system that measures incident and transmitted light at the two different portions of a treatment chamber (e.g., a treatment zone) of a treatment system using light for the treatment of products according to one embodiment of the invention. Furthermore, as illustrated, the detector system measures incident and transmitted light at entrance and exit portions of a treatment chamber.

Illustrated in FIG. 23 are a treatment chamber 1501 (generically, a fluid flow path) having a treatment zone 1704 of a treatment system using light. A fluid product to be treated with the light treatment is flowed through the treatment chamber at a desired rate (in the direction of arrow 2328) while being illuminated with the light treatment. In this embodiment, optical detector 2320 is positioned to view light transmitting through an entrance portion of the treatment zone 1704, optical detector 2322 is positioned to view incident light illuminating the entrance portion of the treatment zone 1704, optical detector 2324 is positioned to view light transmitting through an exit portion of the treatment zone 1704, and optical detector 2326 is positioned to view incident light illuminating the exit portion of the treatment zone 1704. The optical detectors 2320, 2322, 2324, 2326 may be any optical detectors or process monitors described herein. For example in one embodiment, the optical detectors are photodiodes, while in another embodiment, they are fiber optic probes leading to a spectroradiometer. The light treatment provided by one or more light sources (not shown) may be any pulsed or continuous wave light treatment, such as those described herein.

The measurements of the optical detectors 2322, 2324, 2326, 2328 are input to a controller 2330 (or other processor or monitor) for analysis. It is noted that in some embodiments, a spectroradiometer (not shown) is coupled in between the various optical detectors and the controller 2330.

In this embodiment, the intensity or fluence measurements at the entrance portion are compared to the measurements taken at the exit portion. Thus, it can be determined if there is any difference between the absorbed light at the entrance of the treatment zone compared to the exit of the treatment zone. For example, such differences may be used to determine if there is a change or to confirm a change in the concentration of the fluid product being treated across the length of flow path of the treatment chamber (treatment zone 1704). These measurements may also be used to determine if there has been or to confirm a change in the treatment geometry across the length of the treatment zone, e.g., the thickness of the treatment zone is different from the entrance to the exit. In other embodiments, these measurements may also be used to determine changes in properties of the fluid product across the length of the treatment zone, e.g., in blood products to determine a change in the protein concentration from the entrance to the exit. In further embodiments, these measurements may also be used to determine the presence of a buildup of denatured protein or contaminants on the system components across the length of the treatment zone, which may be helpful in determining when replacement treatment chambers may be needed.

By taking the difference between the measurements between optical detectors 2320 and 2322, a level of absorption at the entrance portion is determined, similar to the absorption profiles discussed above. Similarly, by taking the difference between the measurements between optical detectors 2324 and 2326, a level of absorption at the exit portion is determined. These two absorption values are compared to determine the change in absorption from the entrance to the exit portions.

It is noted that in some embodiments, the incident light upon the entrance and exit portions is assumed to be approximately the same, such that the measurements from optical detectors 2322 and 2326 are not used; for example, optical detectors 2322 and 2326 are not present. Thus, the measurements from optical detectors 2320 and 2324 are taken and compared to each other to determine changes in the transmitted light, which will provide the similar benefits of analysis as embodiments using optical detectors 2322 and 2326.

It is noted that in other embodiments, it is not required that the measured and compared locations be at the entrance and exit portions of the treatment zone. Thus, incident and transmitted light is measured at a first location and a second location in order to determine absorption changes across a length of the treatment zone from the first location to the second location. Furthermore, the fluid flowed through the treatment chamber may be a buffer fluid or a fluid product to be treated as described herein.

Referring back to FIG. 22, in broad terms a method according to one embodiment of the invention includes the steps of: illuminating a treatment chamber of a treatment system with a light treatment, the treatment chamber containing a product to be treated with the light treatment (Step 2210). A portion of the treatment chamber and the product transmissive to at least 1% of light having at least one wavelength within a range of 170 to 2600 nm. The light treatment may be any light treatment such as described herein.

Next, a fluence level of a portion of the light treatment transmitting through the treatment chamber at a first location proximate to a first portion of the treatment chamber is measured (Step 2212). Also, a fluence level of a portion of the light treatment transmitting through the treatment chamber at a second location proximate to a second portion of the treatment chamber is measured (Step 2214). The second location is positionally offset from the first location and the first location and the second location within a portion of a profile of the treatment chamber or treatment zone. For example, as illustrated in FIG. 23, optical detectors 2320 and 2324 are positioned at two different locations on the through-side of the treatment chamber that corresponding to two differently located portions of the treatment chamber.

As illustrated in FIG. 23, in one embodiment, the first portion is an entrance portion of the treatment zone while the second portion is an exit portion of the treatment zone. However, it is understood that the first and second portions may be variously located about the profile of the treatment zone.

Generally, this method may be used for a variety of purposes, several of which are described in more detail in the embodiments of the FIGS. 23-25. However, in general terms, the product to be treated may be any product such as described herein, for example a solid or fluid (flowing or static fluid) that is transmissive to a portion of the light treatment. Furthermore, in the event the product is a fluid, the fluid may be a buffer fluid, test fluid, or the fluid product to be treated with the light treatment.

Furthermore, generally, an apparatus to perform the general method of FIG. 22 in the context of the embodiment of FIG. 23 should include a treatment chamber for containing a product to be treated with a light treatment. At least a portion of the treatment chamber and the product are transmissive to at least 1% of light having at least one wavelength within a range of 170 to 2600 nm. Also included are a first optical detector positioned to measure a fluence level of light transmitting through a first portion of the treatment chamber and a second optical detector positioned to measure a fluence level of light transmitting through a second portion of the treatment chamber. The second portion is positionally offset from the first location. In some embodiments, these measurements may be used, for example, to generate a dose mapping of the fluence transmitting through the treatment chamber at various portions or to analyze changes in the absorption or transmission across a length of fluid flow or treatment.

Applied to the embodiment of FIG. 23, the product is a fluid such that the fluid is fluid is flowed through the treatment chamber during the illuminating step (Step 2210). In this embodiment, the method is used to determine changes in the absorption across a portion of the length of fluid flow of a treatment chamber. In one embodiment, the device of FIG. 23 may be used to practice the steps of FIG. 22, although it is understood that other structure may be used to practice this embodiment of the invention. The fluid may be a buffer fluid or a fluid product intended to be treated with the light treatment. The light treatment may be any light treatment as described herein.

Next, after steps 2212 and 2214, the measured fluence levels are compared, for example, by the controller 2330. As described above, these measurements can provide information about changes in the absorption of the fluid along a length of fluid flow within the treatment zone between the first portion and the second portion.

In alternative embodiments in the context of FIG. 23, Steps 2212 and 2214 include measuring a fluence level of the light illuminating the first portion and the second portion of the treatment zone. In this alternative embodiment, the level of absorption is determined at the first and second portions by taking the difference between the measured fluence illuminating the respective portion and the fluence level transmitting through the respective portion of the treatment zone. These absorption levels are then compared to determine changes in the absorption from various points along the flow path.

As described above, changes in the absorption or fluence of transmitted light along the length of the treatment chamber (or treatment zone) from the first portion to the second portion can indicate changes in a property of the fluid product, such as the concentration of the fluid product. These changes can also indicate, for example, changes in the protein concentration in a blood product from the first to the second portions. Furthermore, changes in treatment chamber or zone geometry, such as thickness, can be determined. Additionally, a buildup of denatured materials, e.g., due to excessive use of the treatment chamber, within the treatment chamber may be determined.

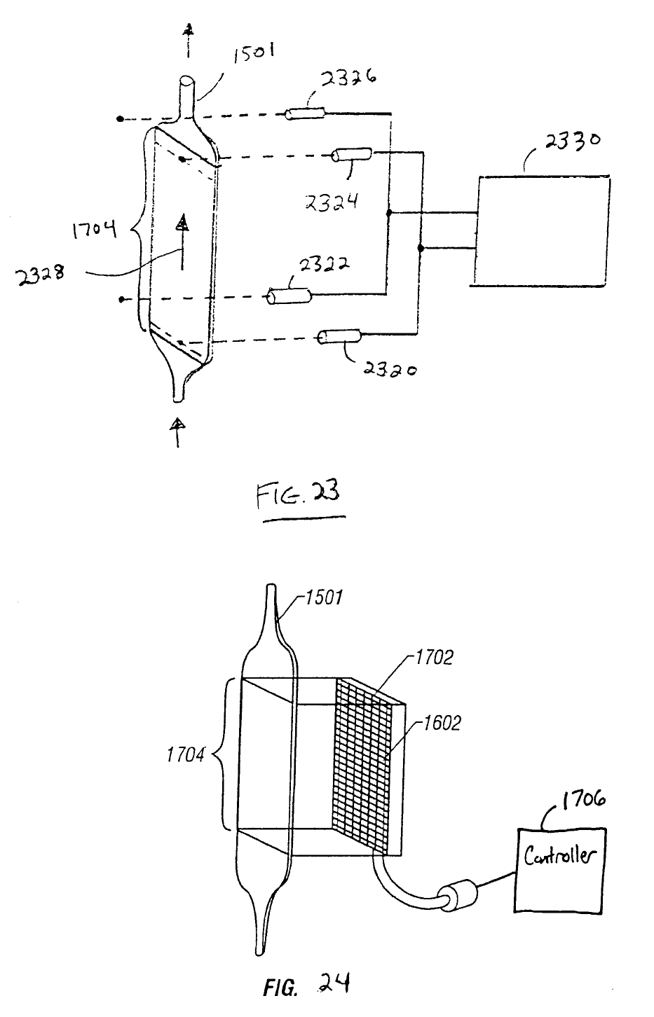

Referring next to FIG. 24, a simplified perspective view is shown of detector array that is used to obtain the spectral profile of the light treatment across the entire treatment chamber according to yet another embodiment of the invention. Illustrated is the treatment chamber 1501, which may be any of the treatment chambers described herein. Rather than two optical detectors to measure the light transmitted through the treatment chamber as described above, e.g., photodetectors or fiber optic probes, a detector array 1702 is positioned behind the entire treatment zone 1704 (which corresponds to at least a portion of the profile of the flow chamber of treatment chamber 1501 in this embodiment) of the treatment chamber 1501. For example, the detector array 1702 is an array of fiber optic probes 1602 arranged in a grid behind the treatment zone 1704 (e.g., arranged on a plate or other structure). In some embodiments, one or more of the fiber optic probes are inputs to one or more spectrometer, e.g., spectroradiometers, that allow the measurement of fluence levels at individual wavelengths within a spectrum of wavelengths at one time. In alternate embodiments, the fiber optic probes 1602 may be other optical detectors or collectors, such as discrete photosensitive devices (e.g., photodiodes) or may comprise a charged coupled device (CCD) array.

The use of the detector array 1702 provides the process controller with the measurements to create a dose mapping of at least a portion of the profile of the treatment zone 1704 (e.g., a profile of the flow chamber) of the treatment chamber 1510. Thus, light energy transmitting through treatment chamber 1501 is collected across a portion of the treatment zone 1704 (or scan area), preferably an entire portion. With no fluid flowing, this detector array 1702 is used to test the uniformity of the light treatment across the portion of the profile of the treatment zone 1704. The same can be tested by pumping a fluid having a known consistent optical density, such as water or another more absorbing fluid. In operation, with the fluid product being pumped through the treatment chamber 1501, the uniformity of the absorption of the light treatment of the fluid is tested. As described, above, particularly with blood plasma derivatives and other bioprocessing fluids, it is important to obtain uniform treatment of the fluid product so that excessive protein damage is prevented while at the same time maximizing the effective treatment of the fluid product, e.g., maximizing the effective kill rate of microorganisms, viruses, bacteria, pathogens, etc..

It is noted that in some embodiments the detector array 1702 may be positioned to measure light passing through at least a portion of the profile of the treatment zone 1704. For example, the detector array structure may be sized smaller than the profile of the treatment zone 1704 or the fiber optic probes 1602 (or other optical detectors) may only cover a portion of the detector array structure. In such embodiments, the detector array 1702 may be sized to measure the light penetrating through less than the entire portion of the flow chamber or treatment zone 1704. Thus, the detector array allows a controller 1706 (or processor or other monitoring device) to create a dose mapping of at least a portion of the profile of the treatment zone 1704 based upon the measurements of the detector array 1702.

In some embodiments, a lens system (not shown) may be positioned between the optical detectors, e.g., photodetectors 1506 and 1508 or fiber probes 1602, and the treatment chamber to focus the transmitted light into the respective process monitor. Such a lens system could comprise a single lens or multiple lenses. Thus, a lens may be positioned in between each optical detector and the treatment chamber. In other embodiments, a lens may be positioned in between the treatment chamber and a CCD array (not shown) in order to focus the energy of the emitted light into the CCD array. In these embodiments, the CCD array is another alternative type of process monitor.

Applying the embodiment of FIG. 24 to the method described thus far in FIG. 22, the structure of FIG. 24 generally follows the same steps. However, additionally, a dose mapping is created for at least a portion of the profile of the treatment chamber based upon the measuring steps (Steps 2212 and 2214). For example, the controller 1706 performs this step.

Again, the light treatment of Step 2210 may be any pulsed or continuous wave light treatment, such as described herein, for the deactivation of microorganisms. The product may be any product to be treated with the light treatment, for example, a solid, liquid or gaseous product. In some embodiments, the product is a fluid product that is flowed through the treatment zone of the treatment system while in other embodiments, the product is stationary within the treatment system and may or may not be a fluid.

However, in this embodiment, the fluence measurements of steps 2212 and 2214 are supplemented by measuring the fluence level of a portion of the light treatment transmitting through the treatment chamber at a plurality of additional locations proximate to additional portions of the treatment chamber. For example, various locations on the detector array structure. As is illustrated, each additional location is generally positionally offset from each other and the first location and the second location. Accordingly, in many embodiments, the various locations for measurements substantially cover at least the portion of the profile of the treatment chamber or treatment zone.

Additionally, as illustrated, the measuring steps, for example, steps 2212, 2214 and the additional measuring steps occur at substantially the same time. This is easily seen since the optical detectors are positioned at various locations on structure of the detector array 1702.

It is noted that when performing the additional step of creating the dose mapping of the profile of the treatment chamber, the profile of the treatment zone may be a profile of the entire treatment zone or a portion of the treatment zone. Generally, the optical detectors are arranged to face an opposite side of the treatment zone as a light source providing the light treatment. These optical detectors may be individual photodetectors or fiber optic probes, for example inputting light to a spectrometer, e.g., a spectroradiometer. It is noted that the fluence measurements may be fluence measurements in the sense that the fluence is present or not, or the fluence measurements may be fluence level measurements at a given wavelength(s).

It is also noted that in one embodiment, the apparatus of FIG. 24 may perform the method of FIG. 22 in use within a treatment system using light for treating products, e.g., deactivating microorganisms as described herein, such as fluid treatment system 100 of FIG. 1. However, it should be understood that these steps may be performed by other light treatment systems having a variety of configurations for treating a variety of products.

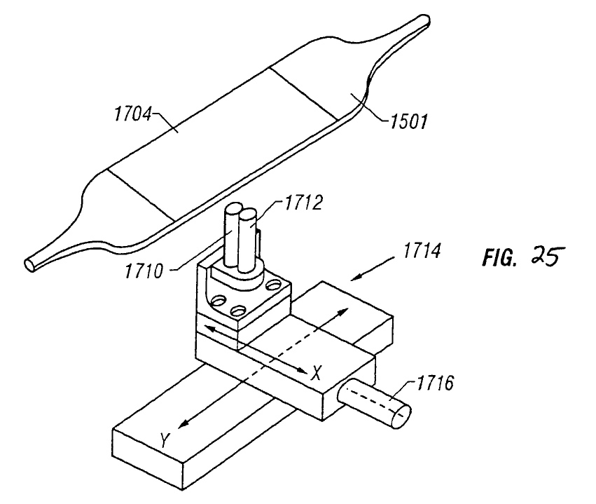

Referring next to FIG. 25, a simplified perspective view is shown of optical detectors integrated on an adjustable x-y translation table used to obtain the spectral profile of the light treatment across different portions of the treatment chamber (or treatment zone) according to yet another embodiment of the invention. In this embodiment, optical detectors 1710 and 1712 are integrated into an x-y translation table 1714, which adjusts the x-y position of the respective optical detectors 1710 and 1712 under the treatment zone 1704 of the treatment chamber 1501. Such x-y translation tables 1714 are well known in the art. The output 1716 allows the process monitor outputs to be coupled to a process controller or other monitoring system for analysis. These optical detectors 1710 and 1712 may be fiber optic probes, photodiodes (or other photodetectors), pressure transducers or thermopiles or other process monitors described herein. In one embodiment, optical detector 1712 is a fiber optic probe (or alternatively, a photodetector) that is configured to measure UV light, e.g., 225-400 nm, while optical detector 1710 is a fiber optic probe (alternatively, photodetector) that is configured to measure light from 400-950 nm. It is noted that these process monitors may be configured to measure light having any specified range of wavelengths or a single wavelength.

The optical detectors 1710 and 1712 are mounted to continuously scan at least a portion of the treatment chamber or treatment zone 1704, e.g., the entire treatment zone 1704, for calibration and/or process monitoring during a fluid run. This would provide additional information relating to the uniformity of the light treatment across the treatment area and may identify areas of fouling and identify areas of the treatment zone that are not being adequately treated.

Applying the embodiment of FIG. 25 to the method described thus far in FIG. 22, the structure of FIG. 25 generally follows the same steps. However, additionally, a dose mapping is created for at least a portion of the profile of the treatment chamber based upon the measuring steps (Steps 2212 and 2214).

Again, the light treatment of Step 2210 may be any pulsed or continuous wave light treatment, such as described herein, for treating the product, e.g., deactivating microorganisms. The product may be any product to be treated with the light treatment, for example, a solid, liquid or gaseous product; however, should transmissive to at least 1% of light having at least one wavelength within a range of 170 to 2600 nm. In some embodiments, the product is a fluid product that is flowed through the treatment zone of the treatment system while in other embodiments, the product is stationary within the treatment system and may or may not be a fluid.

However, the measuring steps 2212 and 2214 are performed differently in this embodiment. For example, an optical detector is positioned at the first location prior to the illuminating step 2210, then the treatment chamber is illuminated. Next, the optical detector is repositioned to the second location and illuminated again. For example, the x-y translation table of FIG. 25 may be used to position the process monitor; however, it is understood that other devices may be used for such positioning. The dose mapping is then generated based upon the fluence measurements of the optical detector positioned at various positions within a portion of the profile of the treatment chamber.

In some embodiments, further steps of repositioning, illuminating and measuring are performed at third, fourth, fifth, and so on locations, to create a higher definition dose mapping. A controller or processor is coupled to the output of the optical detector, and is used to create the dose mapping. As described above, the dose mapping is a plot or mapping of fluence vs. position within the profile of the treatment zone and is useful in determining if the treatment across the treatment zone is uniform. It is further noted that in some embodiments, the measurements of the optical detector may be compared to measurements of the light treatment from an optical detector positioned to measure light that illuminates the treatment zone to determine the dose mapping as an absorption profile across the profile of the treatment zone.

It is further noted that the method of FIG. 22 in the context of the apparatus of FIG. 25 may be performed while flowing a fluid product through the treatment chamber and illuminating the treatment chamber and the fluid product.

Depending on the embodiment and the type of optical detector used, the fluence measurements may be a single measurement indicating the fluence across one or more wavelengths of the light treatment, or the fluence measurement may include multiple fluence measurements across multiple wavelengths of the light treatment collected at the first location.

In one embodiment, the apparatus of FIG. 25 may perform the method of FIG. 22 in use within a treatment system using light for the deactivation of microorganisms as described herein, such as fluid treatment system 100 of FIG. 1. However, it should be understood that these steps may be performed by other light treatment systems having a variety of configurations for treating a variety of products.

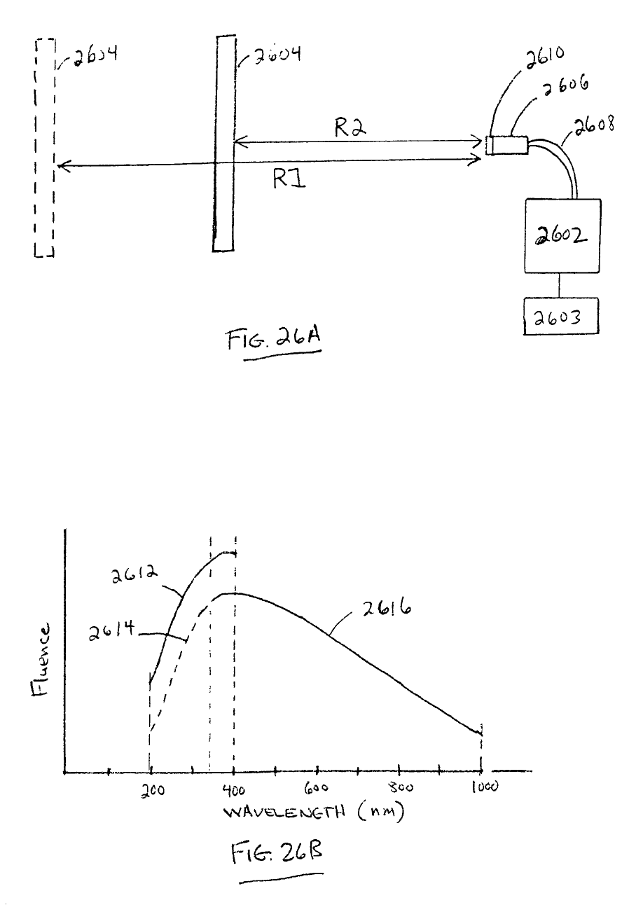

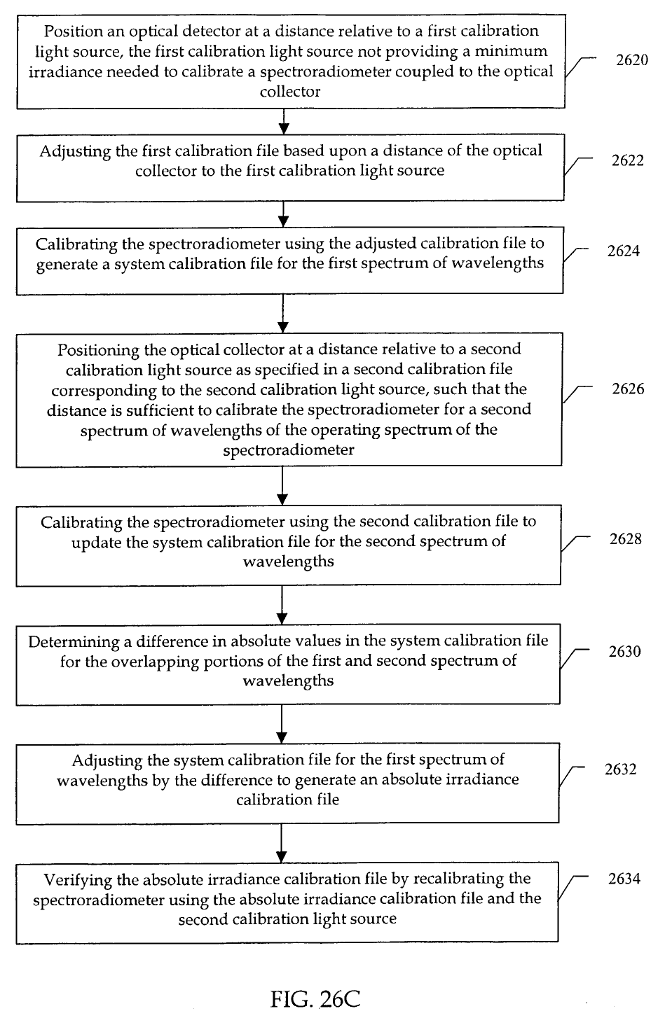

The following description relates to FIGS. 26A-26C. First, referring to FIG. 26A, a diagram is shown illustrating a light source used for the calibration of a spectrometer in accordance with one embodiment of the invention.

According to several embodiments of the invention, a spectroradiometer is used to measure the fluence levels at each of a plurality of wavelengths of a light treatment having a spectrum of wavelengths, e.g., to measure the fluence levels each of the wavelengths. As is known in the art, a spectroradiometer is a device that is commercially available, for example, Model S2000, Miniature Fiber Optic Spectrometer, of Ocean Optics, Inc., of Dunedin, Fla., USA. Typically, in order to perform an absolute irradiance calibration of such a spectrometer 2602, a calibration light source (e.g., lamp) for the range of wavelengths to be calibrated is used. The lamp manufacturer supplies a calibration file to the customer. This calibration file is a listing of fluence levels at each discrete wavelength within the spectral range of the calibration light source. The calibration file is generated by illuminating the spectroradiometer with a standardized light source, for example, a NIST (National Institute of Standards and Technology) traceable light source 2604 (also referred to generically as a “calibration light source”), at a standardized distance and operational settings. For example, a 30 watt deuterium lamp is one example of a NIST traceable UV calibration light source, e.g., the NIST traceable light source 2604 of FIG. 26A. A 30 watt deuterium UV lamp is designed to operate in the far field irradiance pattern of the lamp at a distance of about half a meter.

However, spectroradiometers with small irradiance (or fluence) collection devices, e.g., optical collector 2606, may not be sensitive enough to be calibrated at the standardized distance R1 of the NIST traceable light source 2604. Disadvantageously, the 30 watt deuterium light source is currently the highest irradiance NIST traceable UV source (200-400 nm) available to the user.

To compensate, the user may move the NIST traceable light source 2604 physically closer to the spectroradiometer 2602 than the distance R1 which is required for the calibration file in order to increase the signal strength enough to calibrate the spectroradiometer 2602. In this case, the user adjusts the absolute values of the provided calibration file according to the 1/R² law. This law provides that the irradiance from a point source varies inversely proportionally to the square of the distance from the collector 2606 to the light source 2604 (i.e., distance R2). Thus, as the distance between the collector 2606 and the light source 2604 is reduced, the values of the provided calibration file must be adjusted according to the 1/R² law, where R is the distance from the point light source to the collector.

However, in many cases, as is well understood in the art, particularly where the collector 2606 is moved close enough to the light source such that the light source 2604 does not appear a point source relative to the collector 2606, the 1/R² law adjustment can not be used with accuracy. This brings the collector 2606 (e.g., a fiber optic probe of the spectroradiometer) into the near field of the NIST traceable light source 2604. Thus, the absolute values of the adjusted measured fluence levels will not accurately correspond to the supplied calibration file. However, it is noted that the relative values (spectral shape) of the calibration file will still be correct. That is, the measured fluence vs. wavelength curve will have the same shape, but will be offset by an unknown amount in fluence (see FIG. 26B). As such, the values of the irradiance or fluence can not be accurately calibrated.

According to several embodiments of a treatment system using a light treatment, the light treatment is measured using an optical collector 2606 such as a fiber optic probe coupled to a fiber optic cable 2608 that is coupled to the spectroradiometer 2602. In many embodiments, the light source is a tubular source. Since it is known that fiber optic probes collect only a very narrow angle of incident light, many embodiments employ a diffuser 2610 over the optical collector 2606, e.g., a cosine corrected probe, that allows light incident over a wide range in incident angles to be collected. Alternatively, an integrating sphere, as known in the art, may be used instead of a diffuser as a cosine corrected probe. Furthermore, if the integrating sphere is too large, it may not match with system size constraints. Thus, a diffuser to cosine correct the incident light is preferred in many embodiments. In many of these embodiments, it is important to collect as much light as possible from multiple incident angles in order to accurately measure the light treatment. However, such diffusers attenuate the incident light, which also reduces the sensitivity of the spectroradiometer to the NIST traceable light source 2604. Thus, in some embodiments, the spectroradiometer 2602 would be sensitive enough to be accurately calibrated by the NIST traceable light source 2604 if diffusers were not employed at the optical collectors 2610. Thus, in these embodiments, a spectroradiometer having a diffuser equipped optical collector can not be accurately calibrated using the lamp manufacturer supplied calibration file within an acceptable error.

Referring also to FIG. 26B, a fluence vs. wavelength plot measured in the calibration of the spectroradiometer according to one embodiment of the invention. While referring to FIG. 26B, concurrent reference will be made to FIG. 26C, which is a flowchart illustrating the steps performed in the calibration of a spectroradiometer in accordance with one embodiment of the invention.

Accordingly, in such cases where a calibration light source, e.g., a NIST traceable light source, does not provide a minimum irradiance needed to calibrate the spectroradiometer, two calibration light sources are used for the calibration as follows.

Initially, an optical collector (e.g., optical collector 2606) is positioned at a distance relative to a first calibration light source, the calibration light source (e.g., the NIST traceable light source 2604) not providing a minimum irradiance needed to accurately calibrate a spectroradiometer coupled to the optical collector (Step 2620). Thus, the distance, e.g., distance R2 of FIG. 26A, is close enough to the first calibration light source such that the first calibration light source provides enough signal to calibrate a spectroradiometer coupled to the optical collector for a first spectrum of wavelengths of an operating spectrum of the spectroradiometer. The first calibration light source is positioned closer to the optical collector than specified in a first calibration file for the first spectrum of wavelengths such that the optical collector is positioned in a near field of the first calibration light source. In one embodiment, the first calibration light source is a 30 W deuterium UV lamp used to calibrate a spectrum of 200-400 nm. Thus, in one embodiment, the first spectrum of wavelengths is 200-400 nm and the operating spectrum of the spectroradiometer is 200-1000 nm for example. The first calibration file is the calibration file provided by the manufacturer for the 30 W deuterium lamp for 200-400 nm. In one embodiment, the optical collector is a fiber optic probe that includes a diffuser that attenuates light while, at the same allows light at a wide of incident angles to be collected. Thus, the diffuser causes the spectroradiometer to be accurately calibrated by the first calibration source using the first calibration file.

It is noted that when referring to accurate calibration versus inaccurate calibration, accurate calibration generally means that the measurements obtains are within a specified variance or error of the values specified in the provided calibration file.

Next, the first calibration file is adjusted based upon a distance of the optical collector to the first calibration light source (Step 2622). For example, the first calibration file is based upon the distance R1, while the optical collector is at a distance of R2 from the first calibration light source. Thus, using the relationship that the absolute value of the fluence from a point source is proportional to the square of the distance from the light source. However, it is noted that at the distance R2, the light source does not appear as a point source (i.e., the collector is in the near field of the calibration light source), such that the absolute value adjustment is not accurate. It is noted that Step 2622 may be performed by a controller 2603 coupled to the spectrometer.

Next, the spectroradiometer is calibrated using the adjusted calibration file to generate a system calibration file for the first spectrum of wavelengths (Step 2624). For example, the first calibration light source is illuminated and fluence level measurements are taken at each wavelength within the first spectrum of wavelengths. These values are stored as the system calibration file. Graphically, referring to FIG. 26C, the absolute values of these measurements are no longer accurate; however, the relative value (spectral shape) is accurate. Curve 2612 illustrates a fluence vs. wavelength plot for one embodiment of the first calibration file for a first spectrum of wavelengths of 200-400 nm, while curve 2614 (dashed) illustrates the actual fluence vs. wavelength plot if it could be accurately measured and calibrated. Note that the relative values of curve 2612 are the same as those in curve 2614 (i.e., curve 2612 has substantially the same shape as curve 2614); however, the absolute values between the two curves are different (i.e., curve 2612 is offset in fluence to curve 2614). In one embodiment, controller 2603 at least in part performs Step 2624.

Next, the optical collector is positioned at a distance relative to a second calibration light source as specified in a second calibration file corresponding to the second calibration light source, such that the distance is sufficient to calibrate the spectroradiometer for a second spectrum of wavelengths of the operating spectrum of the spectroradiometer (Step 2626). The second calibration source is another NIST traceable light source. In one embodiment, the second calibration light source is a Quartz Tungsten Halogen lamp (QTH lamp) that has a usable wavelength range of about 350 nm to 1000 nm and beyond. The QTH lamp is available with power ratings of up to 1000 watts. The calibration light source or lamp manufacturer also supplies the second calibration file that was prepared based upon the QTH lamp at distances of about half a meter.

As such, a portion of the second spectrum of wavelengths overlaps a portion the first spectrum of wavelengths, while the first and second spectrum of wavelengths cover the operating spectrum of the spectroradiometer. For example, in one embodiment, the first spectrum is 200-400 nm and the second spectrum is 350-1000 nm, such that there is overlap from 350-400 nm. For example, the operating spectrum of the spectroradiometer may be 200-400 nm, 200-500 nm, 200-1000 nm, 300-500 nm, 350-1000 nm, or any other range depending on the specific spectrometer and that can not be accurately calibrated using a single calibration light source.

Next, the spectroradiometer is calibrated using the second calibration file to update the system calibration file for the second spectrum of wavelengths (Step 2628). For example, the second calibration light source is illuminated and fluence level measurements are taken at each wavelength within the second spectrum of wavelengths. These values are stored in the system calibration file. Graphically, referring to FIG. 26B, the absolute value and relative values (spectral shape) of these measurements should be accurate since the second light source is correctly used as a calibration device. Thus, curve 2616 illustrates a fluence vs. wavelength plot for one embodiment of the second calibration file for a second spectrum of wavelengths of 350-1000 nm. Note that between 350-400 nm, the absolute values and relative values of curve 2616 match that of curve 2614. Note also that between 350-400 nm, the relative values of curve 2616 match that of curve 2612; however, the absolute values are offset. Thus, the absolute values of the overlapping portion of the first spectrum of wavelengths and the second spectrum of wavelengths from the two calibrating steps do not match. In one embodiment, controller 2603 at least in part performs Step 2628.

Then, a difference in absolute values in the system calibration file corresponding to the portion of the first spectrum of wavelengths and the second spectrum of wavelengths that overlap is determined (Step 2630). For example, the difference between the absolute values of curves 2616 and 2612 at one or more of the overlapping wavelengths is determined. In one embodiment, the difference is determined as a ratio between the spectroradiometer reading using the second calibration light source (curve 2616) to the spectroradiomter reading using the first calibration light source (curve 2612) for one or more of the overlapping wavelengths. In some embodiments, the difference is determined in terms of at a single discrete wavelength or as an average of the ratio of multiple discrete wavelengths.

Accordingly, the system calibration file is adjusted for the first spectrum of wavelengths by the difference to generate an absolute irradiance calibration file (Step 2632). For example, the absolute values of the system calibration file for the values obtained in the first spectrum from the first calibration light source are adjusted so that they substantially match those obtained in the overlapping spectrum from the second calibration source. As such, the entire first spectrum of wavelengths, even those portions of the spectrum outside of the overlapping spectrum will match for calibration. In other words, curve 2612 is shifted to approximate curve 2614 such that between 350-400 nm, curves 2612 and 2616 will match both in absolute and relative values. These values are stored in the absolute irradiance calibration file which provides accurate calibration of the spectroradiometer.

Finally, as a final check, the absolute irradiance calibration file is verified by recalibrating the spectroradiometer using the absolute irradiance calibration file (Step 2634). The second calibration light source energy spectrum is then read and compared to the second calibration file for that lamp. These readings must match the absolute irradiance calibration file for both sets of wavelengths within acceptable limits with no discontinuity. This check will ensure that the spectroradiometer is fully calibrated if all measurements match the original second calibration file in the second spectrum. It is noted that in one embodiment, controller 2603 may at least in part perform Steps 2630, 2632 and 2634.

It is noted that generally, different calibration light sources may be used having different spectrums depending on the operating spectrum and sensitivity of the spectroradiometer to be calibrated. However, a single calibration light source may not be available for the accurate calibration of a spectroradiometer across its entire operating spectrum. That is, the supplied calibration file can not be accurately adjusted for the distance of the collector offset from the calibration distance (e.g., R1) in the calibration file for one of the calibration light sources. Thus, a two calibration light source system is used in which one calibration source is used for a portion of the operating spectrum and another calibration light source is used for another portion of the operating spectrum. Only one of the calibration sources is used as directed to calibrate the respective portion of the spectrum, while the portions of the spectrums overlap by one or more wavelengths in order to calibrate the combination of the two spectrums.

Thus, in broad terms, this method may be described as first, calibrating a first spectrum of wavelengths of an operating spectrum of a spectroradiometer with a first calibration light source, the first calibration light source not providing an accurate calibration of the spectroradiometer in the first spectrum of wavelengths. For example, the first calibration light source does not provide an accurate absolute irradiance calibration, but is accurate for the relative values or spectral shape. Second, a second spectrum of wavelengths of the operating spectrum of the spectroradiometer is calibrated with a second calibration light source, the second calibration light source providing an accurate calibration of the spectroradiometer in the second spectrum of wavelengths, and a portion of the first spectrum of wavelengths overlapping the second spectrum of wavelengths. For example, the second calibration light source provides an accurate absolute irradiance calibration. And, third, the calibration of the first spectrum of wavelengths is adjusted based on a difference between the first calibration and the second calibration at the portion of first spectrum of wavelengths overlapping the second spectrum of wavelengths. This results in an absolute irradiance calibration file that is sufficient to calibrate the spectroradiometer across the first spectrum of wavelength and the second spectrum of wavelengths.

It is noted that this method may be implemented variously in the treatment systems using spectroradiometers described herein or other systems in which it is required to calibrate a spectroradiometer. Additionally, the steps of FIG. 26C may be performed in part by a controller 2603 or other processor configured to perform the appropriate adjustments, comparisons and calculations.

It is further noted that in some embodiments, depending on the spectrum desired to be measured in use of the treatment system, two or more spectrometers may be used to adequately cover the operating spectrum of the treatment system. For example, in one embodiment, a first spectrometer is used that has an operating spectrum of 200-500 nm and a second spectrometer is used that has an operating spectrum of 350-1000 nm. It is understood that the exact spectrometers used and their operating spectrums will vary depending on the needs of the system they are used in.

As such, an absolute irradiance calibration, such as described with reference to FIG. 26C or otherwise is performed for each spectrometer (i.e., absolute irradiance calibration files are generated for each spectrometer). An algorithm extracts each wavelength and irradiance information for the bands from each spectrometer and pieces them together into one continuous file. This file is then processed to display the overall spectrum or to calculate the energy in a selected wavelength band. For example, a controller coupled to the two or more spectrometers pieces the various measurements to generate fluence measurements over the entire operating spectrum of the treatment system, e.g., curves such as illustrated in FIGS. 17 and 48 are generated.

Furthermore, as described herein, in many embodiments, one or more spectrometers are used to enable precision light treatment measurements at each wavelength across a spectrum of interest during use of the treatment system. In many embodiments, these measurements are performed in real time, which enables real time control of the various light treatment and system parameters.

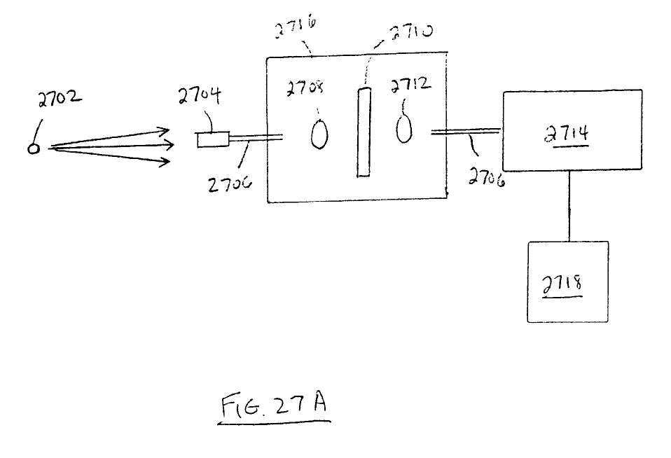

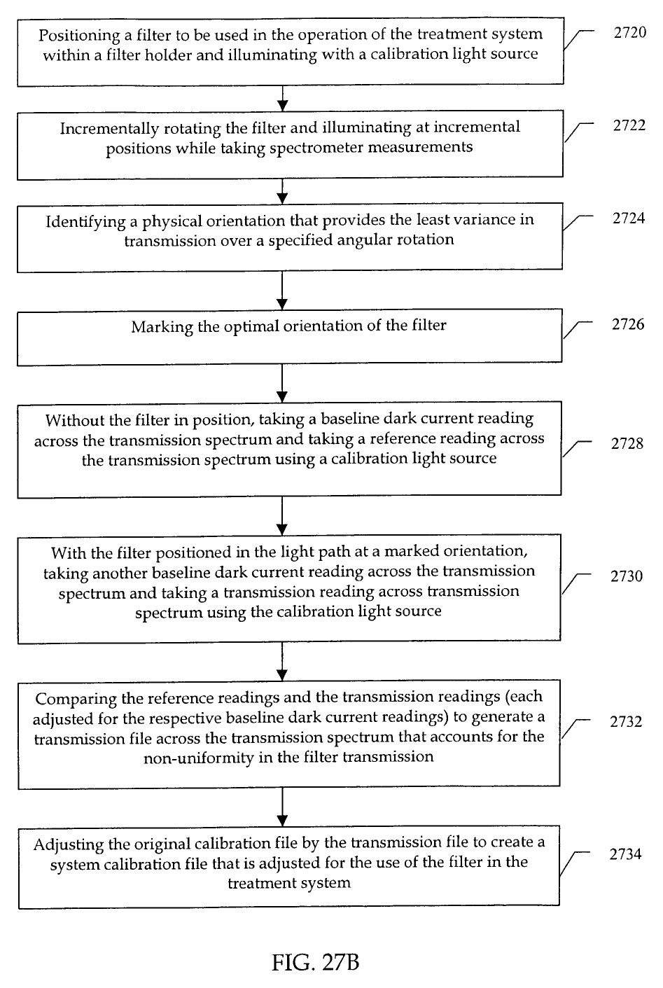

The following description relates to FIGS. 27A-27B. First, referring to FIG. 27A, a diagram is shown illustrating one method of attenuating light received at a spectrometer for use in a treatment system using light for the treatment of products according to one embodiment of the invention.

Illustrated is a light source 2702, an optical collector 2704, fiber optic cable 2706, collimating optics 2708, a neutral density (ND) filter 2710 (also referred to generically as a filter), refocusing optics 2712, a spectrometer 2714 (such as a spectroradiometer), a filter holder 2716, and a controller 2718.

The spectrometer 2714 may be any spectrometer device, such as described herein and may be used in any of the treatment systems such as those described herein.

In many embodiments of a treatment system, a light source is used that is intended to provide illumination within a range of fluences. Also, in these embodiments, a spectrometer device is to be used to measure the fluence of the light treatment, e.g., measure the provided illumination and/or the illumination transmitting through a product being treated. However, in many treatment applications, the fluences to be generated by the light source 2702 are above the fluence rated for use with a given spectrometer 2714. As is well known, this results in the saturation of the detector array (e.g., detector array 1622 of FIG. 16B, which also referred to as the CCD array) of the spectrometer 2714. For example, in one embodiment of a spectrometer, fluence levels of greater than 0.25 J/cm² will result in the saturation of the detector array. Also, in this example, the treatment system uses a light source designed to provide irradiance with a fluence of between about 0.25 to 3.0 J/cm².

As such, a method is provided to attenuate the light input to the spectrometer 2714 while at the same time maintaining the calibration of the spectrometer 2714 (such as calibrated according to FIGS. 26A-26C or otherwise). For example, the calibration should be held within ±1 to 2% of the calibration. It is noted that in many embodiments, the calibration light sources used to calibrate the spectrometer 2714 are typically several order of magnitude dimmer than the light source(s) 2702 used for light treatment and that the spectrometer is configured to measure. In these cases, the spectrometer 2714 is first calibrated, then a means of attenuating the light input to the spectrometer is put into place prior to the actual use of the spectrometer 2714 within the treatment system. However, the means to attenuate the light should be incorporated into the calibration of the spectrometer prior to actual use or the actual values of the spectrometer are adjusted by this calibration.

As illustrated in FIG. 27A, the light source(s) 2702 produces a light treatment, a portion of which is collector by the optical collector 2704, e.g., a fiber optic probe. The light source 2702 may be any light source described herein, such as a pulsed light source or a continuous wave light source producing polychromatic light having fluence levels that would saturate the detector array of the spectrometer 2714 if not attenuated. Fiber optic cable 2706 couples the collected light to the spectrometer 2714.

In order to attenuate the light input to the spectrometer 2714 and prevent saturation of the detector array, a break is made in the fiber optic cable 2706. Collimating optics 2708 are inserted into the light path to collimate the light extending from the fiber optic cable 2706 into a beam (e.g., a beam of about 1-2 mm) which is then passed through the neutral density filter 2710. The ND filter 2710 is held in a desired position or marked orientation within the filter holder 2716. The filtered beam of light then passes through refocusing optics 2712, which refocuses the light beam into the fiber optic cable 2706. The light is then directed to the spectrometer 2714.

As known in the art, an ND filter 2710 passes a portion of incident light within a given transmission spectrum; thus, attenuating the light. For example, a given neutral density filter 2710 may pass 10% of the incident light having a spectrum of 200-400 nm. However, the transmission of standard ND filters 2710 may vary up to ±3% or 4% across the face of the filter. As such, the non-uniformity of the ND filter 2710 transmission leads to non-uniformity in the transmitted light, such that the spectrometer 2714 readings may not be within calibrations. Thus, spectrometer readings that do not account for this non-uniformity may have considerable error. This problem is exacerbated as the width of the spectrum of light to be passed through the ND filter 2710 increases, i.e., the larger the transmission spectrum. For example, in some embodiments, the ND filter 2710 may pass light having wavelengths from 200-300 nm, 200-500 nm, 200-1000 nm, or any other range of wavelengths depending on the exact system. The wider the spectrum to be passed through the ND filter 2710, the wider the area of the ND filter 2710 the light passes through, making non-uniformity of transmission worse.

The problem is also worsened depending on the variation of fluence of the light treatment passing through the ND filter 2710. For example, an ND filter passing light having wavelengths of 240-290 nm may transmit non-uniformly since the fluence levels of the light treatment may be rapidly changing within the transmission spectrum. Thus, a means is needed to account for the non-uniformity of transmission of the ND filter 2710 across the transmission spectrum for accurate spectrometer readings.

Referring next to FIG. 27B, a flowchart is shown illustrating the steps performed to calibrate a neutral density filter 2710 to be used the attenuation of light input to a spectrometer 2714 while maintaining the spectrometer calibration.

The following method is preferably used when a non-uniformly transmitting neutral density filter to be used to attenuate light input to a spectrometer will cause the spectrometer calibrations to vary greater than a predetermined error threshold. For example, in one embodiment, more than ±1 to 2% of the calibration. It is noted that other systems may tolerate larger error, depending on the implementation. Generally, the wider the transmission bandwidth of the ND filter 2710, or the larger the variation of fluences of the light treatment within the transmission spectrum, the more likely this error threshold will be exceeded.

Initially, an ND filter 2710 to be used in the operation of the treatment system is positioned within a filter holder device 2716 and illuminated with a calibration light source (Step 2720). For example, a NIST traceable calibration lamp such as those described with reference to FIGS. 26A-26C may be used. In any event, in many embodiments, the calibration light source provides considerably less fluence that the light source 2702 to be used in the treatment of products. Readings of the illumination are taken from the spectrometer 2714 and input to the controller 2718, for example. In the event the calibration light source is a continuous wave light source, the illumination is for a prescribed period of time to provide proper readings.

Next, the filter 2710 is slowly rotated (incrementally rotated) and illuminated at incremental positions taking spectrometer measurements (Step 2722). The measurements of the spectrometer 2714 are each taken over a prescribed period of time. These measurements are input to the controller 2718 and analyzed to find a “flat spot” in the transmission. That is, a physical orientation that provides the least variance in transmission over a specified angular rotation is identified (Step 2724). At this point, the system is not attempting to find the portion of the ND filter 2710 that most uniformly transmits light, but is attempting to find a portion or area of the ND filter 2710 whose transmission across the transmission spectrum changes very little over a distance of rotation (to ensure repeatability). In preferred embodiments, it is desired to find an optimal orientation of the ND filter 2710 that has a stable transmission over an angular rotation of ±5 degrees.

This optimal orientation of the filter is marked (Step 2726) so that the filter 2710 may be oriented at the optimal location in use. For example, a corresponding mark is made on the filter holder 2716 for alignment. The advantage of finding the optimal orientation is that when the filter 2710 is in use, the filter 2710 may be moved in and out of the light path with little likelihood that if the filter 2710 is inserted into the filter holder 2716 offset slightly from the optimal orientation, that the filter 2710 will still provide substantially the same transmission characteristics.

It is noted that in some embodiments, Steps 2720, 2722 and 2724 are not performed. Alternatively, an arbitrary location if the ND filter 2710 is marked as the filter orientation (alternate Step 2726), such that every time the filter 2710 is used, it is positioned within the filter holder 2716 at that orientation. In order to ensure that the transmission characteristics will remain substantially as determined (see below), care should be taken to make sure that the ND filter 2710 is positioned as close as possible to the marked filter orientation. As a worst case, if the transmission characteristics change rapidly at an angular rotation of the filter 2710 from the marked filter orientation, a slight offset in orientation of the filter 2710 in the filter holder 2716 may result in the transmission characteristics changing enough to introduce error into the spectrometer readings calibrated for the specific ND filter 2710. However, if the ND filter 2710 is carefully aligned at the marked filter orientation, then the spectrometer readings should remain accurate.

It is noted that the method applies whether there is one or more filters 2710. However, in the event there are multiple filters 2710, the orientation of each filter 2710 should be marked, so that it can be calibrated for the orientation of all of the filters 2710.

Next, the ND filter 2710 is removed from the filter holder 2716 (i.e., without the filter in position in the light path) and a baseline dark current reading is taken across the transmission spectrum and a calibration light source is activated to take a reference reading from the spectrometer across the wavelengths of the transmission spectrum (Step 2728). To calibrate the ND filter 2710 as accurately as possible, the baseline dark current response of the spectrometer is taken, i.e., no light is input to the spectrometer and readings are taken from the spectrometer 2714. In one embodiment, all light is blocked to the spectrometer. Even with no light, there are typically baseline dark current readings. The reference reading is the spectrometer reading when the illumination is provided into the spectrometer from the calibration light source without the ND filter 2710 present. Thus, the controller 2718 adjusts the reference reading by the baseline dark current reading.

Then, the filter 2710 is repositioned in the light path (e.g., in the filter holder 2716) at the marked orientation (optimal orientation or other marked orientation) and another baseline dark current reading is taken across the transmission spectrum and the calibration light source is illuminated to take transmission readings from the spectrometer across the spectrum (Step 2730). Thus, the controller 2718 adjusts the transmission reading by the baseline dark current reading. A separate baseline dark current reading is taken since this reading varies with time. Again, these readings in Steps 2728 and 2730 are taken for a specified period of time.

It is noted in some embodiments, the baseline dark current reading is not taken and the controller 2718 simply uses the straight reference readings (without the filter 2710) and the transmission readings (with the filter 2710).

Next, the reference readings and the transmission readings (each adjusted for the respective baseline dark current readings) are compared to generate a transmission file across the transmission spectrum that accounts for the non-uniformity in the ND filter transmission (Step 2732). As such, the transmission file is a file indicating the transmission level of light through the ND filter 2710 for each wavelength of the transmission spectrum. For example, if the light has wavelengths from 200-500 nm, the transmission file indicates how much light is transmitted at each wavelength from 200-500 nm, which also varies from wavelength to wavelength. In one embodiment, transmission readings are divided by the reference readings for each wavelength of the transmission spectrum to generate the transmission file.

This method thus far produces an accurate ND filter transmission file at the resolution level of the spectrometer 2714. Advantageously, once the transmission file is known, the readings of the spectrometer 2714 in use can be compensated variously on a per wavelength basis depending on the non-uniformity of the transmission across the transmission spectrum. Thus, the readings from the spectrometer 2714 in use will be accurate and within an acceptable calibration error.

This compensation may be take place in the spectrometer calibration process, for example, the original calibration file is adjusted by the transmission file to create a system calibration file that is adjusted for the use of the ND filter into the system (Step 2734). For example, the original system calibration file is multiplied by the inverse of the transmission file to create the system calibration file that accounts for the non-uniform transmission of the specific ND filter(s) 2710. Advantageously, in this embodiment, the readings that are taken from the spectrometer in use with the light source 2702 are already calibrated and adjusted for the non-uniform filter 2710 transmission. This enables real time spectrometer measurements of the light treatment. Thus, various controllers may be used to analyze and react to these real time measurements.

Alternatively, the non-uniform transmission of the ND filter(s) may be accounted for as the spectrometer 2714 measures the light treatment, by adjusting the spectrometer measurements on a per wavelength basis based on the transmission file generated in Step 2732. However, this requires extra processing at the controller 2718 or processor coupled to the spectrometer 2714 while these measurements are taken, possibly introducing delay. Thus, preferably, the spectrometer is precalibrated for the use of the ND filter 2710, so that adjustments during measurements are not required in use.

In broad terms, this method may be described as first, generating a transmission file corresponding to a filter (e.g., an ND filter) used to attenuate light input to a spectrometer, the filter non-uniformly transmitting light within a transmission spectrum through the filter, the transmission file generated on a per wavelength basis. For example, in one embodiment, Steps 2720-2732 or Steps 2726-2732 may be performed. Second, the calibration of the spectrometer is compensated based on the transmission file, such that readings of the spectrometer account for non-uniform transmission of the filter on a per wavelength basis. For example, in one embodiment, Step 2734 is performed.

It is noted that this method may be implemented variously in the treatment systems using spectrometers described herein or other systems in which it is required to calibrate a spectrometer. Additionally, the steps of FIG. 27B may be performed in part by the controller 2718 or other processor configured to perform the appropriate adjustments, comparisons and calculations.

### System Operation, Control and Feedback

This section describes many of the operational and control features of several embodiments of the invention, as well as the use of measurements for automated feedback. In many embodiments, the operational and control features utilize the measurements and data collected using one or more of the methods described with reference to FIGS. 15A-27. Furthermore, one or more of the control and feedback features may be incorporated into a controller, such as illustrated in FIGS. 28-44. In many embodiments, the control features described herein are intended to be dynamically used during the treatment process to provide automatic feedback in real time. For example, while a given product is being treated (e.g., a flowing product is illuminated with a light treatment), measurements are taken and an appropriate controller automatically makes adjustments to the light treatment and other system parameters in real time.

Referring next to FIG. 28, a simplified side view is shown of a treatment chamber including a spectral filter positioned between the treatment chamber and the flashlamp according to another embodiment of the invention. Illustrated is the treatment chamber 1802 held in between a first window plate 1804 and a second window plate 1806 defining a thickness of a flow chamber of the treatment chamber 1808 (i.e., defining two dimensional boundaries of the flow chamber). The thickness of the flow chamber is adjustable by adjusting a screw 1810 of a cartridge 1812 (or digital precision spacers or other spacing structure, e.g., spacers 814 of FIG. 8). In order to filter portions of the emitted light from the flashlamp 154, a filter 1814 is positioned in between the flashlamp 154 and the treatment chamber 1802. The filter may be positioned in a variety of ways. For example, referring to FIGS. 1-3, the filter 1814 may be positioned on either side of window 128, or may be positioned within the cartridge 134. For example, referring to FIG. 8, the filter 1814 may be positioned in between the cartridge top 802 and the first window 806. Advantageously, this filter 1814 allows for the selectable spectral filtering of the light from the light source 154. It is noted that the structure that defines the distance between the two windows or plates is positioned outside of the two plates in some embodiments (as shown); however, may be positioned in between the two plates in other embodiments (e.g., a spacer held in position in between first window plate 1804 and a second window plate 1806). Furthermore, in some embodiments, the thickness of the treatment chamber or treatment zone is adjusted automatically in response to light treatment measurements and/or other system measurements and parameters. In these embodiments, for example, screw 1810 is coupled to a drive motor 1811 controlled by a process controller 1813. In response to fluence measurements of transmitted light, the controller 1813 may determine that the treatment thickness should be decreased in order to more uniformly treat the fluid flowing therethrough. A control signal is transmitted to the drive motor 1811, which rotates the screw 1810 a predetermined amount to cause the change in treatment thickness. Such changes in treatment zone thickness may be based upon measurements of flow rate through the treatment chamber, the type of fluid product, the concentration of fluid product to buffer fluid, measured fluence, and changes in the absorption of the product, for example. Furthermore, in some embodiments, the user can input the desired starting treatment chamber thickness, which will then be set by the controller. Thus, in these embodiments, a dimension of the treatment zone is automatically set and can be automatically adjusted during use depending on light treatment, flow and/or system measurements/calibrations.

Referring next to FIG. 29, a simplified side view is shown of a treatment chamber including a device to cool the treatment chamber due to the heat energy of the light illuminating the treatment chamber according to another embodiment of the invention. Although the usage of Xenon flashlamps generates a considerable amount of heat, in many embodiments, means to cool the treatment chamber and the light source 154 is not provided. This is due to relatively short period of time of operation in the completion of a single fluid run. Generally, the treatment chamber does not heat up enough to affect the fluid product.

However, a production scaled version of the fluid treatment system may operate for several hours continuously. Thus, in such systems, a means to cool the treatment chamber is provided. Additionally, the light source 154 itself may be cooled, for example, by pumping water or another liquid through a sheath 1902 surrounding the light source 154. Thus, it is noted that any of the pulsed light sources in the various treatment systems described herein may include an appropriate sheath for flowing a cooling medium therethrough. Likewise, the treatment chamber may be cooled by flowing a cooling medium 1904, such as water or air, through a conduit 1906 or sheet positioned against the transmissive windows holding the treatment chamber 1802. In other embodiments, the cooling is provided by a chill plate, heat exchanger or vortex coolers, fans, or even immersing treatment chamber into a bath of cooling material. Alternatively, in one embodiment, cooling tubes are adhered to the exterior of the windows holding the treatment chamber 1802.

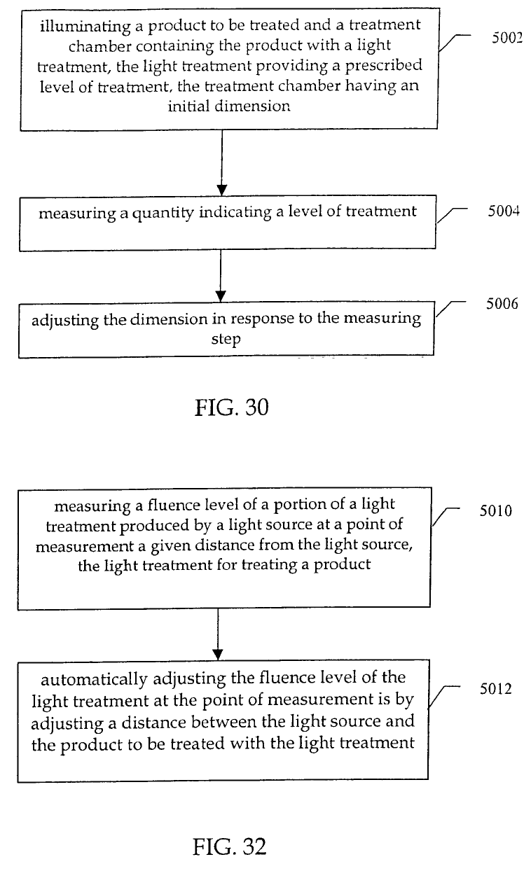

Referring next to FIG. 30, a flowchart is shown illustrating the steps performed according to another embodiment of the invention in which a dimensional boundary of a treatment zone of a treatment system using a light treatment for the deactivation of pathogens (e.g., viruses, microorganisms, etc.) is automatically adjusted. In one embodiment, the steps of FIG. 30 may be performed by the apparatus of FIG. 28 in use within a treatment system using light for the treatment of products as described herein, such as fluid treatment system 100 of FIG. 1. However, it should be understood that these steps may be performed by other light treatment systems having a variety of configurations for treating a variety of products for a variety of purposes.

Initially, a product to be treated and a treatment chamber (or treatment zone) containing the product are illuminated with a light treatment, the light treatment providing a prescribed level of treatment for treating the product, the treatment chamber having an initial dimension, such as a predetermined thickness (Step 5002). The treatment chamber is transmissive to at least 1% of light having at least one wavelength within a range of 170 to 2600 nm. The light treatment may be any pulsed or continuous wave light treatment, such as described herein. Furthermore, the light treatment may be for the deactivation of microorganisms including viruses, bacteria, fungus, etc. of the product, or the light treatment may be otherwise for the modification of the product. The product may be any product to be treated with the light treatment, for example, a solid, liquid or gaseous product. In some embodiments, the product is a fluid product that is flowed through the treatment chamber of the treatment system while in other embodiments, the product is stationary within the treatment system and may or may not be a fluid.

Next, a quantity indicating a level of treatment is measured (Step 5004). Thus, according to one embodiment, the quantity corresponds to a measure of the fluence of the light treatment as measured by an optical detector. Such an optical detector may be any optical detector described herein or known in the art. Thus, the optical detector may output a single fluence measurement at one wavelength, a single fluence measurement for a spectrum of wavelengths, or multiple fluence measurements for each wavelength within a spectrum of wavelengths. In other embodiments, the quantity is based on an absorption profile which is generated by using fluence measurements of a portion of the light treatment illuminating the product and the treatment chamber and a portion of the light treatment penetrating through the product and the treatment chamber.

In other embodiments, the quantity corresponds to measurements of the flow rate of a fluid product through the treatment zone. In further embodiments, the quantity corresponds to a measurement of the concentration of the product within a buffer, e.g., the concentration of a fluid product within a buffer fluid. Such quantities may be determined, for example, by using any of the process detectors, flow rate detectors, etc. that are described herein or any other known detectors or means for determining a quantity relating to the level of the light treatment. It is also noted that the quantity may also be referred to as a system measurement, which may include the types of measurements described above and also includes other system measurements, such as flow pressure, treatment chamber temperature and density of the product.

Next, the dimension of the treatment chamber is automatically adjusted in response to the quantity in order to maintain a prescribed level of treatment (Step 5006). In one embodiment, the thickness of the treatment chamber is automatically adjusted. Such adjustment is accomplished by a controller coupled to the treatment chamber that generates the appropriate control signal to cause the dimension of the treatment chamber to be adjusted. In the embodiment of FIG. 28, for example, in response to fluence measurements of transmitted light, a controller may determine that the treatment thickness should be decreased in order to more uniformly treat the fluid flowing therethrough. This controller generates a control signal that is transmitted to a drive motor, which rotates the screw a predetermined amount to cause the change in treatment thickness. In other embodiments, depending on the configuration, other dimensions of the treatment chamber may be automatically adjusted, such as, the width, length and/or volume of the treatment zone. It is noted that the adjusting step may be performed during the light treatment process, e.g., while continuously performing the illuminating and measuring steps.

It is particularly important in several embodiments to control the thickness of the treatment chamber for flowing products having a laminar flow profile. For example, if the thickness is too large, then particles farthest from the light source across the treatment thickness will not be treated to the same degree as fluid particles closer to the light source across the treatment thickness, resulting in non-uniform treatment of the product. As such, indicators, such as measured fluence transmitting through the product, flow rate, and concentration help to indicate how uniform the light treatment may be across the dimension of the treatment chamber.

Referring next to FIG. 31, a simplified view of an adjustable fluence light treatment system is illustrated according to one embodiment of the invention. Shown are a treatment chamber 3002 of the light treatment system, light source 3004 positioned to illuminate the treatment chamber 3002, a positioner 3006 coupled to the light source 3004 and for moving the light source 3004 to change the distance of the light source to a product to be treated. For example, in the embodiment shown, the positioner 3006 is a linear positioner that positions the light source 3004 at a selectable position along a linear axis 3008 extending toward the treatment chamber 3002 that is adpated to contain the product. Also illustrated are optical detectors 3010 and 3012, and a controller 3014 coupled to the positioner 3006 and the optical detectors 3010, 3012.

The adjustable fluence system of FIG. 31 provides for the automatic adjustment of the fluence level of light that illuminates the treatment chamber 3002 and products contained therein or flowed therethrough. In operation, the light source 3004 illuminates the treatment chamber 3002 with a light treatment that is for the purpose of treating products within the treatment chamber 3002. However, it is important that the fluence be carefully controlled to ensure that too much fluence does not illuminate the treatment chamber 3002 and the product. This is particularly true for sensitive biological compounds, such as blood plasma derivatives. A careful balance between the kill rate (log reduction of the pathogen) vs. damage to the product itself due to overtreatment is to be obtained.

Initially, if the operator wishes to treat the target at a given fluence level, the controller 3014 generates the appropriate control signal to the positioner 3006, which moves the light source 3002 to the appropriate distance from the treatment chamber 3002. Typically, the fluence levels of the light treatment at each position (or step) of the positioner 3006 are measured and stored prior to use. Thus, the controller 3014 can determine what position the positioner 3006 should place the light source relative to the treatment chamber 3002 in order to provide the desired fluence or treatment level. As described above, advantageously, an adjustment of the linear distance from the light source 3004 to the treatment chamber 3002 or product provides for a uniform fluence adjustment without affecting the spectrum of the light treatment. In several embodiments, this adjustment occurs during the treatment of the product, e.g., while the product is being illuminated.

According to one embodiment, once the position of the light source 3004 relative to the treatment chamber 3002 is set, the optical detectors 3010, 3012 continue to measure the fluence level of the light treatment that illuminates the treatment chamber 3002 and transmits through the treatment chamber 3002 and the product within. These measurements are stored over time and if it determined that the measured fluence levels of the light treatment change, e.g., decline, then the controller 3014 generates the appropriate control signal to cause the positioner 3006 to reposition the light source 3004 along the linear axis 3008 until the measured fluence levels are at the desired level. Advantageously, this method compensates for aging in the light source 3004 and reflector assembly 3016 (if used).

In other embodiments, the fluence level of the light treatment is adjusted in response to other system determinations (for example, made at the controller 3014 or other feedback and control system). In one embodiment, the control system may determine that the fluence level of the light treatment is too high or that the desired fluence level should be otherwise reduced to meet other desired treatment parameters. Accordingly, the controller 3014 generates the appropriate control signal to reposition the positioner 3006, e.g., repositioning the light source 3004 at another position along the linear axis 3008.

The system of FIG. 31 may be implemented in a treatment system such as that illustrated in FIG. 11, such that the positioner 3006 is embodied as the linear slide servo drive 112, which may be referred to generically as an automated linear slide. In other embodiments, for example, in any other embodiments described herein, the positioner 3006 is a stepper motor or a magnetic drive. However, the adjustable fluence system may be implemented in other treatment systems having other geometric configurations, as long as the system provide for an automatic adjustment of the linear distance between the light source 3004 and the treatment chamber 3002 or product. For example, several light sources may be positioned radially about a cylindrical treatment chamber, such that the position of each light source is radially adjustable closer and further from the treatment chamber (i.e., the linear distance of each lamp to the product is adjusted). Furthermore, in some embodiments, the positioner 3006 moves the light source 3004 about an axis that does not extend toward the treatment chamber 3002 or the product; however, movement along this axis nevertheless alters the linear distnace between the lgiht source and the product. One example includes moving the light source along an axis perpendicular to axis 3008 as illustrated.

It is noted that the controller may be integrated with the functionality of one or more of the controllers and control systems described herein. It is also noted that the light source may be one or more light sources such as described herein, for example, continuous wave or pulsed light sources. It is further noted that depending on the embodiment, the fluence level of the light treatment illuminating the treatment chamber may be adjusted for many reasons, some of which are explored further below.

It is also noted that in some embodiments, the product to be treated is not required to be within a treatment chamber. In these embodiments, the distance between the light source and the product is controlled by the controller 3014. Furthermore, it is not required that both optical detectors 3010 and 3012 be present. For example, the distance adjustments may be made based on the measurements of either detector or another detector located at any other known position. In some embodiments, the detector is positioned within the product to be treated, e.g., within a fluid product.

Referring next to FIG. 32, a flowchart is shown illustrating the steps performed according to another embodiment of the invention in which the fluence level of a light treatment for treating a product is adjustable by an automatic adjustment of the distance of the light source to a product to be treated. In one embodiment, the steps of FIG. 32 may be performed by the apparatus of FIG. 31 in use within a treatment system using light as described herein, such as fluid treatment system 100 of FIG. 1. However, it should be understood that these steps may be performed by other light treatment systems having a variety of configurations for treating a variety of products.

Initially, a fluence level of a portion of a light treatment produced by a light source is measured at a point of measurement a given distance from the light source, the light treatment for treating a product (Step 5010). In one embodiment, the product is contained within a treatment chamber, although it is not required that the product be contained within a treatment chamber. In one embodiment, an optical detector is positioned at the point of measurement and the point of measurement is located to collect light at a position at or proximate to the treatment chamber or product. Thus, in some embodiments, the fluence level of a portion of the light treatment produced by the light source that illuminates the product is measured. In another embodiment, the product is a fluid product contained within a treatment chamber, and preferably flowed through the treatment chamber. When the product is within the treatment chamber, the treatment chamber is transmissive to at least 1% of light having at least one wavelength within a range of 170 to 2600 nm.

The light treatment may be any pulsed or continuous wave light treatment, such as described herein, for treating the product. For example, the light treatment is for the deactivation of microorganisms. The product may be any product to be treated with the light treatment, for example, a solid, liquid or gaseous product.

Next, the fluence level of the light treatment at the point of measurement is automatically adjusted by adjusting a distance between the light source and the product to be treated with the light treatment (Step 5012). For example, an optical detector positioned to view light at the point of measurement outputs a signal to a controller, the signal corresponding to the fluence level of the light treatment at the point of measurement. In response, the controller determines that an adjustment of the fluence of the light treatment is needed and generates a control signal. The control signal causes a positioner to reposition the light source either closer or farther from the product being treated. For example, the light source is moved along an axis that alters the distance of the light source to the product. In some embodiments, the automatic adjustment occurs during the treatment of the product.

In one embodiment, the automatic adjustment is for the purpose of compensating for the aging of the light source and/or reflector assembly of the light source. For example, as the light source and reflector age, the fluence levels output will gradually decline. The automatic adjustment compensates by positioning the light source slightly closer to the product in order to maintain a uniform treatment level. In other embodiments, the automatic adjustment is to simply maintain a fluence level of the light treatment at a constant level regardless of the causes of increases or decreases in measured fluence levels, again, providing a uniform treatment level of the product.

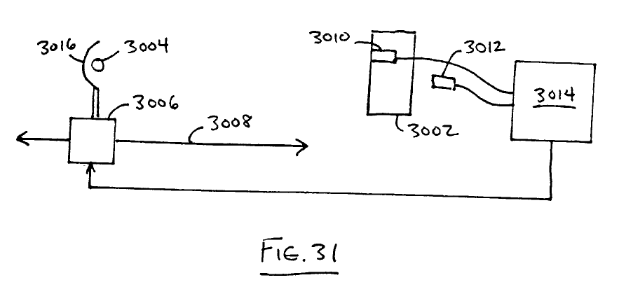

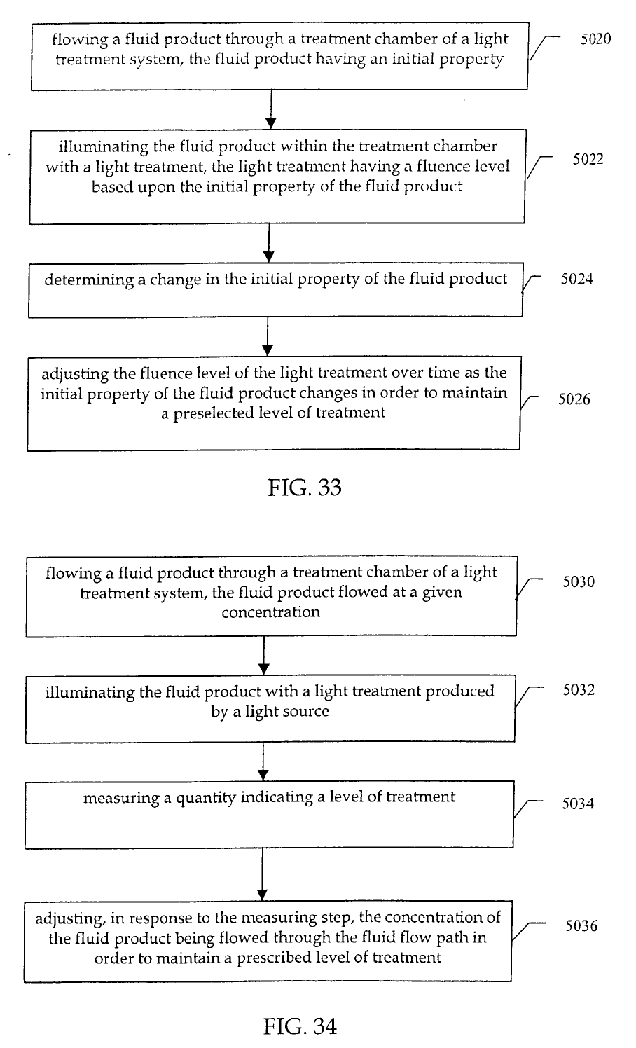

Referring next to FIG. 33, a flowchart is shown illustrating the steps performed according to another embodiment of the invention in which the fluence level of a light treatment for treating a product is adjustable according to property changes in the product being treated. In one embodiment, the steps of FIG. 33 may be performed by any of the apparatii in use within a treatment system using light as described herein, such as fluid treatment system 100 of FIG. 1. However, it should be understood that these steps may be performed by other light treatment systems having a variety of configurations for treating a variety of products.

Initially, a fluid product is flowed through a treatment chamber (or a fluid flow path) of a light treatment system, the fluid product having an initial property (Step 5020). Preferably, the treatment chamber is transmissive to at least 1% of light having at least one wavelength within a range of 170 to 2600 nm. The initial property may be any property or characteristic of the fluid product, for example, the concentration of the fluid product within a buffer fluid, the density of the fluid product and the opacity of the fluid product.

Next, the fluid product is illuminated with a light treatment, the light treatment having a fluence level based upon the initial property of the fluid product (Step 5022). One or more light sources produce the light treatment. The light treatment may be any pulsed or continuous wave light treatment, such as described herein, for treating the fluid product. For example, the light treatment is for the deactivation of microorganisms within the fluid product. It is noted that the one or more light sources may be located within the treatment chamber or external to the treatment chamber where the treatment chamber is transmissive to at least a portion of the light treatment.

Next, it is determined if the initial property has changed (Step 5024). This determination may be made as a result of system measurements and/or light treatment measurements. For example, in some embodiments, a portion of the light treatment illuminating the product and/or a portion of the light treatment transmitting through fluid product are measured, for example, using optical detectors appropriately positioned. Based upon changes in such measurements, it can be determined whether the initial property has changed. In further embodiments, an absorption profile of the fluid product is determined based upon the measuring the incident and transmitted light, the absorption profile indicating a quantity of the light treatment absorbed by the fluid product. Changes in the absorption profile indicate that the property has changed. For example, if the concentration of the fluid product within a buffer fluid is reduced, less light should be absorbed.

In other embodiments, changes in the property may be determined by system measurements. For example, the amount of a buffer fluid metered into the flow path with the fluid product may be changed, which is then fed to the controller. The controller then knows that the property has changed.

Next, the fluence level of the light treatment is adjusted over time (during the flowing) as the initial property of the fluid product changes in order to maintain a preselected level of treatment (Step 5026). In preferred embodiments, the adjustment is an automatic adjustment is response to system measurements and/or light treatment measurements. In many embodiments, the fluence level is adjusted by automatically adjusting a distance from the one or more light sources providing the light treatment to the product, such as variously described herein. Advantageously, by adjusting the distance of the light source(s) to the product, the fluence is uniformly adjusted across the spectrum of wavelengths of the light treatment.

The determining and adjusting steps are typically performed by a controller that receives various system and light treatment measurements. The controller then generates the appropriate control signals that are transmitted to the components of the treatment system to cause the appropriate adjustments. This functionality may be embodied in any of the controller devices described herein.

Referring next to FIG. 34, a flowchart is shown illustrating the steps performed according to another embodiment of the invention in which the concentration of a fluid product within a buffer fluid to be treated with a light treatment is automatically adjustable according to light treatment measurements. In one embodiment, the steps of FIG. 34 may be performed by any of the apparatii in use within a treatment system using light as described herein, such as fluid treatment system 100 of FIG. 1. However, it should be understood that these steps may be performed by other light treatment systems having a variety of configurations for treating a variety of products.

Initially, a fluid product is flowed through a treatment chamber (or a fluid flow path) of a light treatment system, the fluid product flowed at a given concentration within a buffer fluid (Step 5030). In some embodiments, the treatment chamber is transmissive to at least 1% of light having at least one wavelength within a range of 170 to 2600 nm.

Next, the fluid product is illuminated with a light treatment, the light treatment produced by one or more light sources (Step 5032). The light treatment may be any pulsed or continuous wave light treatment, such as described herein, for treating the fluid product. For example, the light treatment is for the deactivation of microorganisms within the fluid product. It is noted that the one or more light sources may be located within the treatment chamber or external to the treatment chamber where the treatment chamber is transmissive to at least a portion of the light treatment.

Next, a quantity indicating a level of treatment is measured (Step 5034). For example, one or more optical detectors positioned to view one or more portions of the light treatment are used to collect measurements of the light treatment. In one embodiment, a portion of the light treatment illuminating the product and/or a portion of the light treatment transmitting through fluid product are measured in order to determine the quantity. In further embodiments, an absorption profile of the fluid product based upon the difference between measurements of the incident and transmitted light measurements is determined, the absorption profile indicating the level of the light treatment or the quantity of the light treatment absorbed by the fluid product.

Next, in response to the measuring step (Step 5034), the concentration of the fluid product being flowed through the treatment chamber is automatically adjusted in order to maintain a prescribed level of treatment (Step 5036). For example, the amount of a buffer fluid metered into the flow path with the fluid product may be changed, which will adjust the concentration of the fluid product. In one embodiment, a controller coupled to the one or more optical detectors determines that an adjustment to the concentration of the fluid product is required (e.g., the fluid product is too concentrated, and thus, the portions of the fluid product farther from the light source(s) are not being treated to the same degree as portions closer to the light source(s)). The controller generates and transmits the appropriate control signals to cause the concentration of the fluid product to be adjusted. For example, in the treatment system of FIG. 1, a control signal is sent to the actuator assemblies 106 and 108 to control the flow of fluid product and buffer fluid appropriately. Any of the controller devices described herein may be configured to analyze light treatment measurements and automatically cause changes in the concentration of the fluid product. A system implementing such a controller should have one or more light sources, a treatment chamber for flowing the fluid product, one or more detectors to measure a portion of the light treatment, a controller, and means to adjust the concentration of the fluid product within the fluid flow.

The following description relates to FIGS. 35A-35C and FIGS. 36-37. As was previously described and illustrated, in many embodiments, the linear position of a light source relative to the product to be treated is adjustable, i.e., the light source(s) may be located at various distances from the product being treated. Furthermore, in many embodiments, it is desirable to know the exact fluence delivered to a product, particularly sensitive biological products in which it is desired not to excessively damage the product due to the light treatment. One problem is that in many embodiments, due to physical limitations, it is difficult to position an optical detector (e.g., a photodetector or fiber optic collector) at a location where it will measure the exact fluence delivered to the product. The following description provides methods to calculate the fluence delivered to the product based upon fluence measured at one or more reference locations or points.

Generally, the method involves a calibration and an implementation. Referring to FIG. 35A, a light source 3502, a location 3504 where a product to be treated is to be located, a collector 3506 at reference point A, a collector 3508 at reference point B, and a controller 3509 are illustrated. The distance between collector 3506 (reference point A) and the location 3504 is x1, the distance between collector 3506 (reference point A) and the light source 3502 is x2, and the distance between collector 3508 (reference point B) and the light source 3502 is x3. The collector 3506 at point A is preferably located along an axis perpendicular to the light source 3502 and extending through the location 3504 on a through side of the location 3504.

As illustrated, when locating the product at location 3504, it is difficult to position an optical detector or collector at that location in order to determine the exact fluence delivered to the location 3504. Generally, collector 3506 collects a portion of the light treatment transmitting through a light transmissive product, while collector 3508 collects the portion of the light treatment incident upon the product. However, while the measurements at collector 3508 approximate the fluence delivered to the product, these measurements do not represent the exact fluence delivered to the product since the collector 3508 (point B) is not positioned at the exact location of location 3504. For example, the collector 3508 is offset from location 3504 in both x and y directions. Furthermore, collector 3506 measures the light treatment transmitting through the product, not the light treatment incident on the product. It is noted that collector 3506 is not used for measurements in the event the product is opaque to the light treatment.

Thus, prior to positioning the product at location 3504 (i.e., there is no product in between the light source 3502 and collector 3506), a fluence vs distance curve is generated. That is, the light source 3502 is positioned at incremental distances from reference point A and activated. In other words, the light source 3502 is positioned at various distances x2 so that measurements of the fluence at each distance x2 can be recorded. It is noted that the light source 3502 may be a pulsed light source or a continuous wave light source. In one embodiment, a pulsed light source is flashed at each incremental distance x2, while in other embodiments, a continuous light source is activated for a prescribed period of time at each distance x2.

FIG. 35B illustrates fluence vs distance curves generated for collectors 3506 at various distances x2 and 3508 at corresponding various distances x3. In one embodiment, curve 3510 is the curve generated from measurements taken at collector 3506 (point A) for various distances x2, while curve 3512 is the generated from measurements taken at collector 3508 (point B) at various distances x3 corresponding to the distances x2. Note that at the largest value of distance x2 (of curve 3510), the largest value of distance x3 (of curve 3512 is smaller; however, this assumes that reference point B is closer to the light source 3502 than reference point A. It is noted that generally, at a given distance x2, the fluence measured at collector 3506 is lower than the fluence measured at collector 3508. However, such is not always the case, especially if collector 3508 is offset far enough in the y direction from collector 3506, such that although collector 3508 is closer to the light source 3502, collector 3508 collects less fluence along the entire length of the light source 3502. It should be understood that this further depends on the range of collection angles that the collectors can input. Thus, it is noted that depending on the configuration, the collectors used, and the various distances involved, the degree to which curves 3510 and 3512 differ will vary.

Next, curve fits are programmatically generated to create equations for both fluence as a function of distance and distance as a function of fluence for both collectors 3506 and 3508. The coefficients for these equations are stored in a file as calibration data for later use. It is noted that the controller 3509 coupled to the collectors 3506 and 3508 is used to make the above determinations and trigger the appropriate repositioning and testing of the light source 3502. It is further noted that it is not necessary to actually plot the fluence vs distance curves, but only that the calibration data (i.e., fluence at various distances) be obtained to model the various equations.

Optionally, a fluence as a function of fluence relationship may be derived by comparing the fluence as a function of distance and distance as a function of fluence equations for the respective curves 3510 and 3512. As such, e given fluence level of curve 3510 corresponds to a given fluence level of curve 3512. For example, given the fluence measured at reference point B, the fluence present at location of the product may be determined, without having to know the specific distance.

In the implementation stage, this calibration data (e.g., from the equations modeled after the fluence vs distance curves) is used to select the starting position of the light source 3502 relative to the product at location 3504. As such, the starting fluence is input by the operator or otherwise known for the treatment. The distance as a function of fluence equation based upon curve 3510 is used to set the distance of the light source 3502 to the product at location 3504. For example, if the fluence desired is F1, then the F1 is input into this equation to yield the appropriate distance x2. However, the distance x2 has been previously defined as the distance from the light source 3502 to the reference point A. Thus, the system sets the distance from the light source 3502 to the location 3504 (and the product 3514) as x2, which is illustrated in FIG. 35C. This accomplished by positioning the light source 3502 a distance of x2+x1 from reference point A (collector 3506), such that the distance from location 3504 to the light source 3502 is now x2. Thus, the starting position of the light source 3502 is a distance of x2+x1, x2 determined from the input fluence in the distance as a function of fluence equation generated from curve 3510.

Next, in use, a method is provided to verify that the exact fluence delivered to the location 3504 is in fact the fluence as was measured prior to the treatment in the generation of curve 3510. As illustrated in FIG. 35C, collector 3506 at reference point A will not provide accurate measurements and verification of the fluence delivered since it now measures the fluence of light transmitting through the product 3514. Likewise, if the product is not transmissive to the light treatment, the collector at reference point A is not used at all.

In order to verify this fluence, the equation modeled from curve 3512 for reference point B is utilized. For example, once the distance from the light source to location 3504 is known, the distance x3 is also known. Thus, in use, the distance x3 is used in a fluence as a function of distance equation modeled from curve 3512 to generate what the expected fluence should be at reference point B (collector 3508). If the measured fluence at reference point B matches the expected fluence, this indicates that the fluence delivered to location 3504 should be substantially equal to the fluence as originally measured at reference point A for the distance x2 (in FIG. 35A). If the measured fluence is not at the expected fluence, then the fluence delivered to the product 3514 will not be equal to that originally measured at collector 3506.

If the measured fluence is not at the expected level, then the system controller may make adjustments to the light treatment, e.g., power to the light source, distance from the product, etc. to bring the measurements to the expected values. In one embodiment, the ratio of the measured to expected fluence at reference point B is used to determine a scaled adjustment to the distance x2 (in FIG. 35C), for example, accounting for aging of the light source (and reflector) or other degradations in the light treatment.

The verification process is performed at various points in time throughout the use of the system. For example, the verification is continually performed or performed at discrete intervals.

It is noted that the fluence measured may be a discrete fluence measurement taken at a single wavelength or a discrete measurement covering light within a spectrum of wavelengths, e.g., a UV measurement. Alternatively, the fluence measurements may be at a plurality of wavelengths across a spectrum of wavelengths. In such cases, the fluence at a selected one of the plurality of wavelengths is used to generate the fluence vs distance curves. In other cases, the fluence measurement or level is an integration of the fluences across a spectrum of wavelengths.

As such, in some embodiments, the collectors 3506 and 3508 are fiber optic probes, each coupled to an appropriate spectrometer, e.g., a spectroradiometer. Preferably, the fiber optic probes are cosine corrected to receive light at a wide input angle. In additional embodiments, two collectors are used at each reference point A and B, each collector collecting light at different wavelengths or within different spectrums. In one embodiment, one collector at each reference point collects UV light (e.g., 200-400 nm) while the other collector at each reference point collects light from 400-1000 nm. Furthermore, each collector at each reference point is input to a respective channel of a two-channel spectroradiometer.

Additionally, the product 3514 to be treated at location 3504 may be a solid or fluid (liquid or gas) product. Furthermore, the product may be transmissive to at least a portion of the light treatment or may be opaque to the light treatment.

Additionally, the location of the reference points may be varied with respect to each other. For example, the collector at reference point B may be further from the light source than the collector at reference point A, although the collector at reference point B should be positioned to receive light from the light source during the treatment of the product. Furthermore, the collector at reference point A may alternatively be positioned at a location closer to the light source than the location 3504. In such alternative embodiments, the collector is used primarily for the generation of the fluence vs distance calibration curves and is subsequently removed for treatment of the product.

It is also noted that the product may be positioned within a treatment chamber having a defined volume. In some embodiments, the product is a fluid product flowed through the treatment chamber. Thus, the treatment chamber may be similar to any of the treatment chambers as described herein or other types of treatment chambers known in the art.

Advantageously, by employing the above calibration and implementation, it is possible to estimate the fluence level of a light treatment at a specified location without requiring an optical detector at that location. This provides the advantage that a collector is not required to be located at the desired location, since it could interfere with the light treatment of the product.

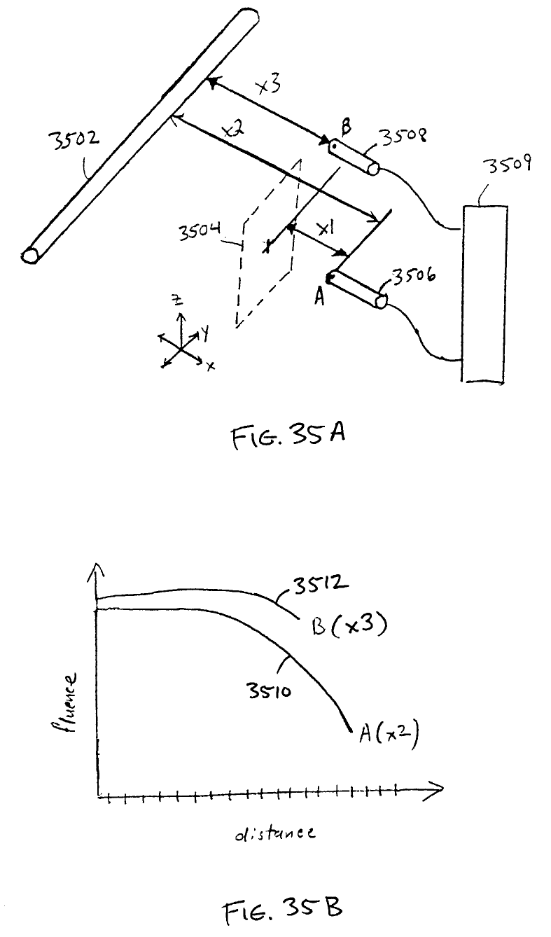

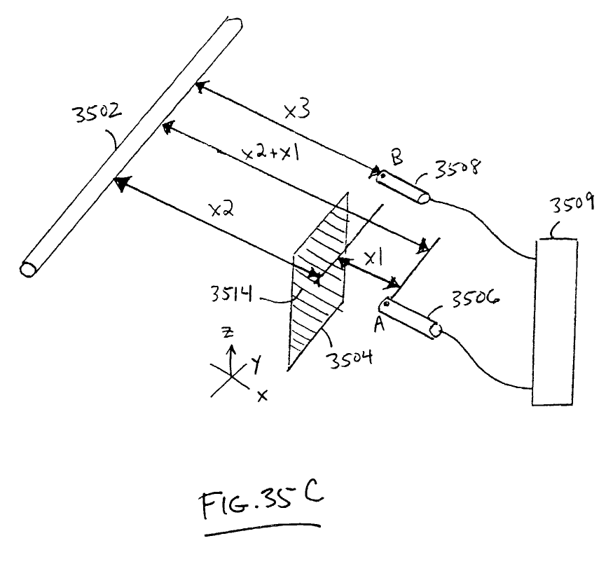

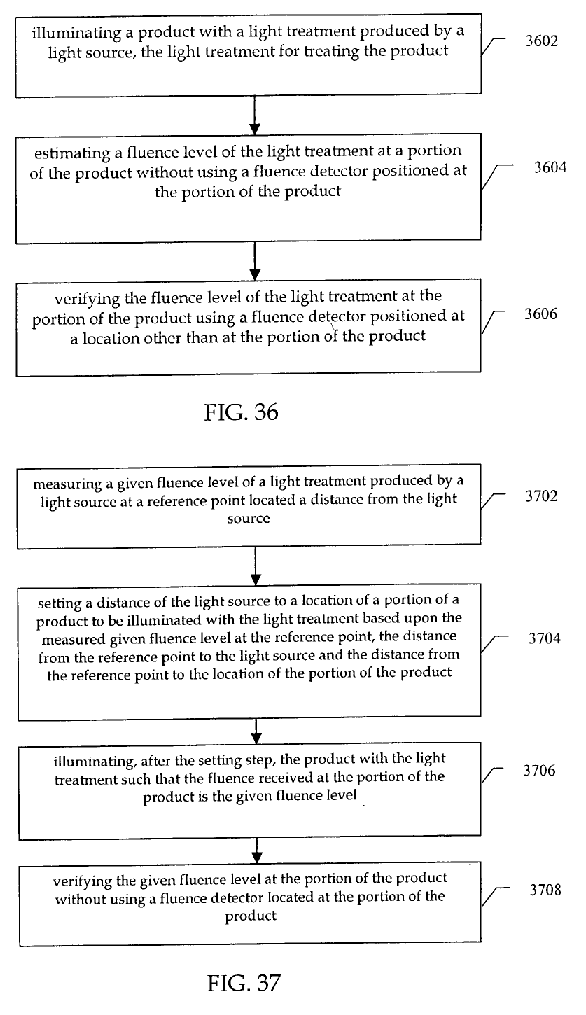

Referring next to FIG. 36, a flowchart is shown illustrating the steps performed according to another embodiment of the invention. In one embodiment, the steps of FIG. 36 may be performed by any of the apparatii, for example, as illustrated in FIGS. 35A and 35C, in use within a treatment system using light as described herein, such as fluid treatment system 100 of FIG. 1. However, it should be understood that these steps may be performed by other light treatment systems having a variety of configurations for treating a variety of products.

Initially, a product is illuminated with a light treatment produced by a light source, the light treatment for treating the product (Step 3602). The light treatment may be produced by one or more light sources, such as described herein. The light treatment comprises light having at least one wavelength within a range of 170 to 2600 nm. The product may be any product, such as described with reference to FIGS. 35A-35C or otherwise herein. The light treatment may be for any purpose such as described herein, for example, for the deactivation of microorganisms. Furthermore, the product may be located within a treatment chamber. Depending on the embodiment, the one or more light sources may be located within the treatment chamber or external to the treatment chamber where the treatment chamber is transmissive to at least a portion of the light treatment.

Next, a fluence level of the light treatment is estimated at a portion of the product without using a fluence detector positioned at the portion of the product (Step 3604). As described above, in several embodiments, this is accomplished by essentially premeasuring the fluence that should be expected at a given distance from a light source, positioning the portion of the product at the location, and then verifying that the premeasured fluence remains accurate. It is noted that the fluence may be measured by the appropriate optical detectors as described with reference to FIGS. 35A-35C or otherwise herein. It is also noted that the fluence level may be a discrete fluence measurement taken at a single wavelength or a discrete measurement covering light within a spectrum of wavelengths. Furthermore, the fluence level may be the fluence at a selected one of the plurality of wavelengths is used to generate the fluence vs distance curves. In other cases, the fluence level is an integration of the fluences across a spectrum of wavelengths.

Furthermore, in one embodiment, the fluence level estimate is based upon fluence as a function of distance from the light source. For example, knowing the distance from the portion of the product to the light source, the fluence seen at the portion of the product is estimated. In another embodiment, the fluence level estimate is based upon fluence as a function of fluence measured at a reference point. For example, knowing the fluence measured at a reference point (e.g., reference point B), the fluence seen at the portion of the product is estimated.

Thus, next, the fluence level of the light treatment is verified at the portion of the product using a fluence detector positioned at a location other than at the portion of the product (Step 3606). In one embodiment, as described above, the verification is based upon premeasurements of fluence at a second reference point which are compared to actual fluence measurements in use at the second reference point.

It is noted that a controller coupled to one or more optical detectors is configured to perform Steps 3604 and 3606.

In one embodiment, similar to that described with reference FIGS. 35A-35C, Step 3604 may be performed by positioning the light source at a first position a first distance (e.g., x2) from an optical detector positioned at a reference point (e.g., collector 3506 at point A). In one embodiment, the first distance is along an axis between the light source and a position (e.g., location 3504) where the portion of the product would be located during treatment. The reference point is located a second distance (e.g., x1) along the axis from the position where the portion of the product would be located during treatment. The light source is located a third distance (e.g., x2−x1) along the axis from the position where the portion of the product would be located during treatment.

Next, the optical detector positioned at the reference point is illuminated with the light treatment having a prescribed fluence level. This prescribed fluence level is verified at the given distance x2.

Next, the light source is repositioned to a second position at the first distance (e.g., x2 in FIG. 35C) from the position where the portion of the product would be located during treatment (e.g., location 3504). It is noted that this distance is now the first distance plus the second distance from the light source to the reference point (e.g., x2+x1).

Once, the light source is repositioned, the product to be treated is positioned, such that the portion of the product is at the position for treatment.

Once the product is positioned, the product is illuminated with the light treatment having prescribed fluence level, such that the fluence level at the portion of the product is substantially equal to the prescribed fluence level.

Referring next to FIG. 37, a flowchart is shown illustrating the steps performed according to another embodiment of the invention for determining the starting position of a light source relative to a product to be treated. In one embodiment, the steps of FIG. 37 may be performed by any of the apparatii, for example, as illustrated in FIGS. 35A and 35C, in use within a treatment system using light as described herein, such as fluid treatment system 100 of FIG. 1. However, it should be understood that these steps may be performed by other light treatment systems having a variety of configurations for treating a variety of products.

Initially, a given fluence level of a light treatment produced by a light source is measured at a reference point located a distance from the light source (Step 3702). The light source and light treatment may be any light source or treatment as described herein. In one embodiment for example, a collector (e.g., collector 3506 of FIG. 35A) positioned at reference point (e.g., point A) that is a distance (e.g., x2) measures the given fluence level. In preferred embodiments, it is noted that a product to be treated with the light treatment is not present at the time of the measuring step.

Next, a distance of the light source to a location of a portion of a product to be illuminated with the light treatment (e.g., x2 in FIG. 35C) is set based upon the measured given fluence level at the reference point, the distance from the reference point to the light source, and the distance from the reference point to the location of the portion of the product (Step 3704). As such, if the given fluence is desired to provided to the portion of the product, the light source is positioned at a distance x2 (in FIG. 35C) from the portion of the product, such that the light source is at a distance x2+x1 from the reference point. Thus, the fluence at the portion of the product will equal the given fluence.

It is noted that the product may be any product such as described with reference to FIGS. 35A-35C or otherwise herein. For example, the product may be a fluid product that is flowed through a treatment chamber. The light source may be located within the treatment chamber or may be external to the treatment chamber, such that the treatment chamber is transmissive to at least a portion of the light treatment.

Next, after the setting step (Step 3704), the product is illuminated with the light treatment such that the fluence received at the portion of the product is the given fluence level (Step 3706). Depending on the embodiment, the product may be illuminated with at least one pulse of light or with a continuous exposure to light. Then, the given fluence level is verified at the portion of the product without using a fluence detector located at the portion of the product (Step 3708).

According to one embodiment, the reference point is located is at a position along an axis extending through the location of the portion of the product and the light source. Furthermore, in this case, the setting step (3704) includes moving (or repositioning) the light source a distance (e.g., x1) substantially equal to the distance from the reference point (e.g., point A) to the location of the portion of the product (e.g., location 3504), such that the distance from the light source to the location of the portion of the product is substantially equal to the distance between the reference point and the light source prior to the moving the light source.

The methods of FIGS. 36 and 37 may be performed by the structure and in the context of that illustrated and described with reference to FIGS. 35A-35C; however, such methods may be performed relative to the structure and context of other light treatment systems in accordance with this embodiment of the invention.

Referring next to FIG. 38, a simple diagram is shown illustrating various particle velocities (velocity vectors) across a thickness of a fluid flow path of a treatment chamber according to one embodiment of the invention.

As described throughout this specification, certain types of fluid products, either static or flowing, are sensitive to the pulsed light treatments. For example, it is often desired to treat the product, such as to deactivate microorganisms without excessively damaging the product itself. This is particularly important in the case of sensitive biological products, such as blood products. The light treatment should be such that a minimum kill rate is obtained without exceeding a maximum level of protein damage, while treating the product as quickly as possible.

In treatment systems that treat flowing fluid products, the flash rate of the pulsed light source is important to meeting this objective. For example, the rate of fluid flow through a treatment chamber affects the rate at which the pulsed light source discharges in order to meet a desired level of treatment. Furthermore, the geometry of the treatment chamber 3802 or fluid flow path effects the velocity of the fluids therethrough. By way of example, across the thickness 3804 (or gap) of a treatment chamber 3802 relative to the light source, particles of the fluid flow at different velocities depending on the location across the thickness of the treatment chamber. Generally, the velocity of particles across the thickness has a parabolic profile illustrated as particle velocity profile 3808. For example, as illustrated in FIG. 38, fluid particles flowing along the boundary of the treatment chamber 3802 flow slightly slower than particles flowing through the central portion of the treatment chamber volume. Note that relative velocity is illustrated in FIG. 38 according to the magnitude of the arrow for a given fluid particle. Each arrow representing a particular velocity vector. Line 3806 indicates the centerline velocity, which is typically the peak particle velocity within the fluid flow. Additionally, it is noted that the particles near the boundary flow slower than, and the particles in the central portion flow faster than, the mass flow rate 3810 (or average flow rate) of the fluid entering the treatment chamber.

Disadvantageously, if the flash rate of the pulsed light source is set based upon a mass flow rate 3810 (average flow rate) of the fluid entering the treatment chamber, some fluid particles have the potential to be undertreated since they will be flowing faster than the mass flow rate.

The variance in treatment at the particle level will be different depending on the geometry of the treatment chamber. It is noted that some systems have an automatic or manual treatment chamber geometry adjustment, or simply provide for the replacement of a treatment chamber with another chamber having a different geometry. In systems that provide for adjustable treatment chamber geometries, such as thickness, as the geometry changes, the degree to which some particles may be undertreated varies.

Additionally, the viscosity of the fluid product also affects the variances in particle flows across the thickness of the treatment chamber. The mass flow rate of the fluid is another factor. For example, altering the velocity at which a fluid is flowed through the treatment chamber may affects the variances in particle flow velocities within the fluid flow.

Accordingly, a method is provided to estimate a particular particle flow velocity within a fluid flow through a treatment chamber, and then set the flash rate based on the particular particle flow velocity, rather than based on the mass flow rate. In some embodiments, and depending on the degree to which the operator is concerned with undertreatment, the particular flow velocity may be that of the fastest particles within the fluid flow, i.e., the peak velocity. Alternatively, the flash rate may be set based upon a particular particle flow rate that is a percentage of the peak particle flow velocity. For example, the flash rate may be based upon 80% of the peak particle flow velocity.

As such, the particular velocity that the flash rate is based upon may be at locations along the particle velocity profile 3808 other than at the peak, i.e., other than at the centerline 3806. In one embodiment, the optical characteristics of the fluid product (e.g., absorption) and the depth of the thickness are considered to select an appropriate particle velocity along the particle velocity profile 3808.

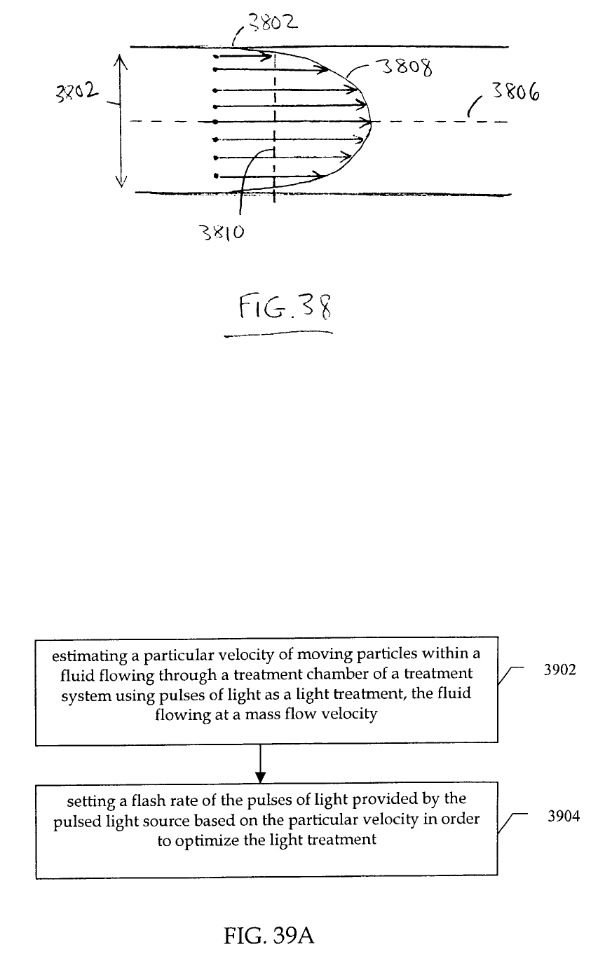

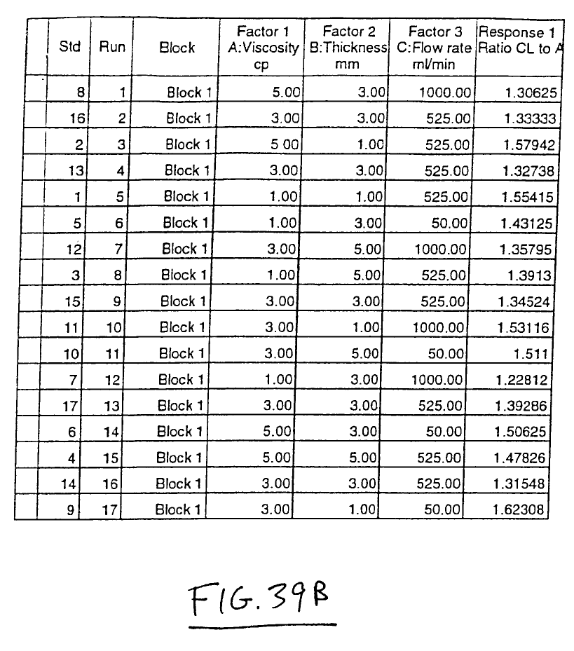

Referring next to FIG. 39A, a flowchart is shown illustrating the steps performed in another embodiment of the invention, which is used to set the flash rate of a pulsed light source treatment system. As such, according to one embodiment of the invention, a particular velocity of moving particles within a fluid flowing through a treatment chamber of a treatment system using pulses of light as a light treatment is estimated, the fluid flowing at a mass flow velocity (Step 3902). In some embodiments, the treatment chamber and the fluid are transmissive to at least 1% of light having at least one wavelength within a range of 170 to 2600 nm. The light treatment may be any monochromatic or polychromatic pulsed light treatment, such as those described herein. The particular velocity may be the fastest particle velocity (peak particle velocity), the slowest particle velocity, or other particular particle flow velocity. In some embodiments, the particular particle flow velocity is a ratio of a centerline velocity (typically the peak particle velocity) to the mass flow rate (average flow rate).

Once the particular particle velocity is estimated, a flash rate of the pulses of light provided by the pulsed light source is set based on the particular velocity in order to optimize the light treatment (Step 3904). In some embodiments, the flash rate is set based a ratio centerline to mass flow rate.

In embodiments in which the undertreatment is a concern, the flash rate is set to treat the fastest moving particles in order to ensure a minimum level of treatment, set based on the peak particle velocity. However, it is noted that the flash rate may be set based upon particular particle flow velocities other than the peak velocity vector depending on the implementation. It is noted that in one embodiment, the optical characteristics of the fluid product and the depth of the thickness are considered to select an appropriate particle velocity along a particle velocity profile in order to meet a minimum level of treatment.

In order to determine the particular particle flow velocity (Step 3902), in one embodiment, a design of experiment (DOE) tool is used to develop an equation for the particular particle flow velocity as a function of several input variables. In one embodiment, the design of experiment tool generates a single equation to model the particle velocity profile given three input variables: fluid viscosity (i.e., a fluid characteristic), treatment chamber thickness (i.e., a flow geometry), and a mass flow rate of the fluid through the treatment chamber. It is noted that other fluid characteristics and flow geometries may be used to model different equations. In one variation, the single equation generated for the particle velocity profile is expressed in terms of a ratio of the centerline (i.e., peak velocity) to average velocity. Thus, the velocity vectors are normalized to the average velocity.

Initially, a battery of tests is run in which the fluid viscosity, treatment chamber thickness and mass flow rate are altered between three selectable values. In each test, the ratio of the centerline to average velocity (CL to A) is determined, either calculated or measured. Alternatively, a particular particle velocity is calculated or measured. In one embodiment, such velocities may be measured by inserting dyes into the flow and using optical sensors or doppler sensors to determine the desired ratio or velocity.

In one example, FIG. 39B illustrates a table of the run conditions of the tests. Note that the fluid viscosity was varied between 1, 3 and 5 cP, the treatment chamber thickness was varied between 1, 3, and 5 mm, and the mass flow rate was varied between 50, 525 and 1000 ml/min. The resulting ratio of the centerline to average velocity varied from about 1.3 to 1.6. Thus, initial finite element analysis, presented in FIG. 39B illustrates that variances in viscosity, thickness and mass flow rate affect the centerline to average velocity.

It is important in some embodiments that the flash rate be set according to the actual ratio of the centerline to average velocity (CL to A) in use. For example, if an operator were to set the flash rate based on the highest CL to A (about 1.6) out of a range of operating CL to As, then when the system actually operates at a CL to A of 1.3, portions of the fluid product would be overtreated. On the other hand, if the flash rate was set based on the lowest CL to A (about 1.3) of the range of operating CL to As, then when the system actually operates at a CL to A of 1.6, portions of the fluid product will be undertreated. Thus, it is important in some embodiments to determine which actual particular velocity or which actual CL to A the fluid is flowing at, and dynamically adjust the flash rate accordingly.

In order to avoid having to run the tests for every conceivable set of operating conditions, design of experiment (DOE) software is utilized to develop a single equation from the data of FIG. 39B. Such DOE software is well known in the art, and one example is commercially available from Stat-Ease Corp. of Minneapolis, Minn., USA. The single equation has as inputs the fluid viscosity, treatment chamber thickness, and mass flow rate and outputs the ratio of the centerline to average velocity. Thus, depending on the specific inputs, the resulting particular velocity, e.g., particular ratio of the centerline to average velocity is estimated. Such software could also have been configured to determine a particular particle velocity rather than the ratio depending on the data entered into the DOE software.

As such, in use, depending on the fluid viscosity, the treatment chamber thickness and the mass flow rate, any one or more of which may be adjusted during according to other aspects of the system control, the ratio of the centerline to average velocity may be determined in real time. Thus, the flash rate of the pulsed light source may be adjusted as other system parameters change to ensure the appropriate level of treatment.

In alternative embodiments, the flash rate may be set based on one or more of the fluid viscosity, the treatment chamber thickness or the mass flow rate may be adjusted. For example, rather than adjust the flash rate, the mass flow rate is adjusted such that the particular velocity is increased or decreased to better match the flash rate. In another example, the viscosity of the fluid product is adjusted, e.g., by metering in more or less of a buffer fluid. Furthermore, the flash rate and one or more of the fluid viscosity, the treatment chamber thickness or the mass flow rate may be adjusted together.

It is noted that the system controller may be adapted to solve the equation from the DOE software to determine what adjustments are needed and then make the appropriate adjustments. For example, the controller will send the appropriate control signals to adjust the flash rate, etc.

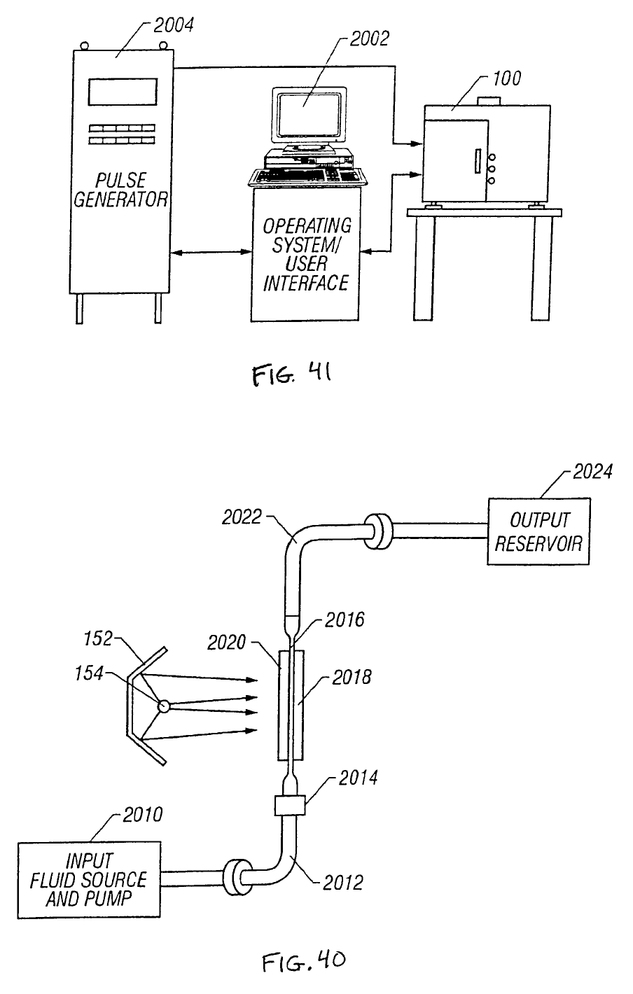

Referring next to FIG. 40, a simplified schematic drawing is shown of a production fluid treatment system scaled to continuously treat fluids. A constant fluid source 2010 is coupled to an input tube 2012 (supply conduit). The constant fluid source 2010 may be a large fluid reservoir or any container having a pump or pumping mechanism to provide the fluid flow at a specified rate. A flow rate detector 2014 may be incorporated into tube 2012 to detect the rate of the fluid flow. The detected flow rate may be used to set the flash rate of the light source 154 (in pulsed light embodiments). It is noted that a flow rate detector may also be coupled to output tube 2022 to measure the flow rate of fluid exiting the treatment chamber 2016. In some embodiments, it is important to maintain a constant flow rate, for example, since the flash rate is set based on the flow rate. Thus, the flow rate detector 2014 serves a function to constantly measure and verify that the flow rate remains at the expected constant level, otherwise the flash rate will not provide the proper treatment levels. Thus, flow rate detectors may be used in any of the embodiments described herein to measure and verify a constant flow rate.

It is also noted that various detectors may be positioned in the fluid flow path at the entrance and exit of the treatment chamber 2016, such as the thermodetectors and pressure transducers described with reference to FIG. 6. The fluid flows through the treatment chamber 2016, which may be similar to those described throughout this specification. The treatment chamber is positioned between two light transmissive support structures (plates or windows 2018 and 2018). These structures define at least one dimensional boundary of the treatment chamber 2016. These structures may be integrated into a cartridge as described above or be separate structures adapted to hold the treatment chamber in position. Additionally, the distance between the plates may be made variable using spacers or spacing structures, screws, for example, under manual or electronic control, such as described herein. Furthermore, in some embodiments, these plates may be used to conform the flow chamber of the treatment chamber to a substantially uniform flow geometry. The treated fluid continues to flow out of the treatment chamber through output tube 2022 and into the output reservoir 2024. This embodiment may require cooling of the treatment chamber depending on the fluence of the flashlamp 154 and the number of flashes, for example. As such, cooling mediums, such as water or air could be circulated over the light transmissive plates 2018 and 2020.

It is noted that in alternative embodiments, the treatment chamber 2016 is not positioned between structures or plates, it is simply positioned to receive light from the light source 154. The treatment chamber 2016 may be flexible or rigid and is preferably removable and disposable. The flow through the treatment chamber 2016 may be any geometry and may provide a flat flow, a laminar flow, a tubular flow, a uniform flow, or a turbulent flow, for example, or any flow as dictated by the dimensions of the treatment chamber (or as dictated by the structures 2018 and 2020 restraining the treatment chamber 2016 and defining at least one dimensional boundary of the treatment chamber 2016). Advantageously, since the treatment chamber is disposable, it may be replaced periodically, rather than having to clean or sterilize it. It is noted that one or more of the data monitoring methods and structure described herein, e.g., with reference to FIGS. 15A-27, as well as one or more of the control related methods and structure described herein, e.g., with reference to FIGS. 28-39B, may be implemented in the system of FIG. 40.

Referring next to FIG. 41, a system level diagram is shown for a fluid treatment system using a light treatment according to one embodiment of the invention. Illustrated are the fluid treatment system 100, the computer operating system/user interface 2002 and the pulse generator 2004. The computer operating system/user interface 2002 includes the main processing and control system/control software to control and operate the fluid treatment system 100 and the pulse generator 2004. It is noted that the system of FIG. 41 may be used to treat any products, fluid or otherwise with any light treatment described herein, e.g., a pulsed light treatment.

The user is able to set the specific parameters for the fluid treatment device for its operation, e.g., one or more of the pump rate (fluid flow rate), the spectral distribution of the light, the fluence or intensity of the light treatment, exposure (e.g., number of flashes or exposure time for a particular portion of the fluid), thickness of treatment zone, concentration of fluid product to buffer fluid, etc. The operating system/user interface 2002 also receives feedback and monitoring signaling from the fluid treatment system 100 as well as controls the pulse generator 2004. The pulse generator 2004 generates the pulses to be delivered to the fluid treatment system 100 and is produced as PUREBRIGHT Model No. PBS-1 available from PurePulse Technologies, Inc. of San Diego, Calif., USA. The pulse generator 2004 includes a pulsing device that includes a DC power supply that charges energy storage capacitors; a switch used to discharge the capacitors; a trigger circuit used to fire the switch at pre-programmed time intervals; and a set of high voltage coaxial cables carrying the discharge pulses from a capacitor-switch assembly to the flashlamp within the housing fluid treatment system 100.

It is noted that one or more of the data monitoring methods and structure described herein, e.g., with reference to FIGS. 15A-27, as well as one or more of the control related methods and structure described herein, e.g., with reference to FIGS. 28-39B, may be implemented in the system of FIG. 41.

According to one embodiment, the control system of the computer operating system/user interface 2002 is a computer-based system that inputs settings as desired by the user and automatically implements these settings for the appropriate test or treatment.

The control system uses feedback from use to implement the settings, or to fine tune the settings during use, or to maintain the settings throughout the test or treatment. In some embodiments, the control system operates fully automatically in response to initial user input. Additionally, the control system stores all test data and performs analysis of the results.

Depending upon the embodiment and level of control offered by the control system, the user may enter as initial test variables one or more of the following: flow rate of the fluid product, the number of flashes a particular portion of the product should be exposed to (generally, the exposure time), the viscosity of the fluid product, the density of the fluid product, the media to be treated, the concentration of the media or product, the microorganism to be deactivated, the inoculation level and the fluence/flash (generally, the dosage).

In response to the user's inputs, the control system translates the input parameters into values usable by the hardware of the light treatment system. For example, the system determines the charge voltage of the light source, the flow rate, the exposure (or flash rate), the distance from the light source to the product or treatment zone, and the thickness of the treatment zone that will implement the user settings. These values are automatically generated and transmitted to the various system components.

In some embodiments, the control system receives calibration data, e.g., measurements taken during use in order to implement or maintain the system settings. For example, in one embodiment, before a product is flowed through the treatment zone and illuminated, the treatment zone is illuminated with a flash of light. In preferred embodiments, a spectrometer, e.g., a spectroradiometer, is used to determine measurements of the fluence and spectrum of the emitted light, which are fed back to the control system of the computer operating system/user interface 2002 to verify the proper parameters and to make the necessary changes to implement the proper settings. It has been found that minute changes in the cleanliness of the system components in the treatment zone as well as aging of components, such as the light source and reflector, etc., may slightly affect the actual measured parameters. In some applications, particularly in applications treating sensitive biological products, precise treatment is required in order to achieve the proper inoculation level without overtreating or damaging the fluid product itself.

In many embodiments, the process controllers described herein, for example, with reference to FIGS. 15A-27 are implemented in the functionality of the control system. For example, many of the analysis and processing steps are performed in software by the control system of the computer operating system/user interface 2002, e.g., generating absorption profiles, determining treatment changes during use, determining system cleanliness, and determining if operational conditions have been met to list a few.

In some embodiments, the treatment system and integrated control system is part of a test system that allows for the automatic adjustment of many input variables in order to eventually determine the optimal set of parameters that will effectively treat a given product. It has been found that different fluid products, particularly, sensitive biological products react differently with different fluences, wavelengths, treatment chamber thickness, number of exposures, etc. and such a system is used to determine what is the optimal fluence, the optimal thickness, the optimal flow rate, the optimal concentration, etc. for a given product. Once these optimal treatment parameters are determined for a given product, then a production scale system may be provided that is specifically designed for the exact product of interest.

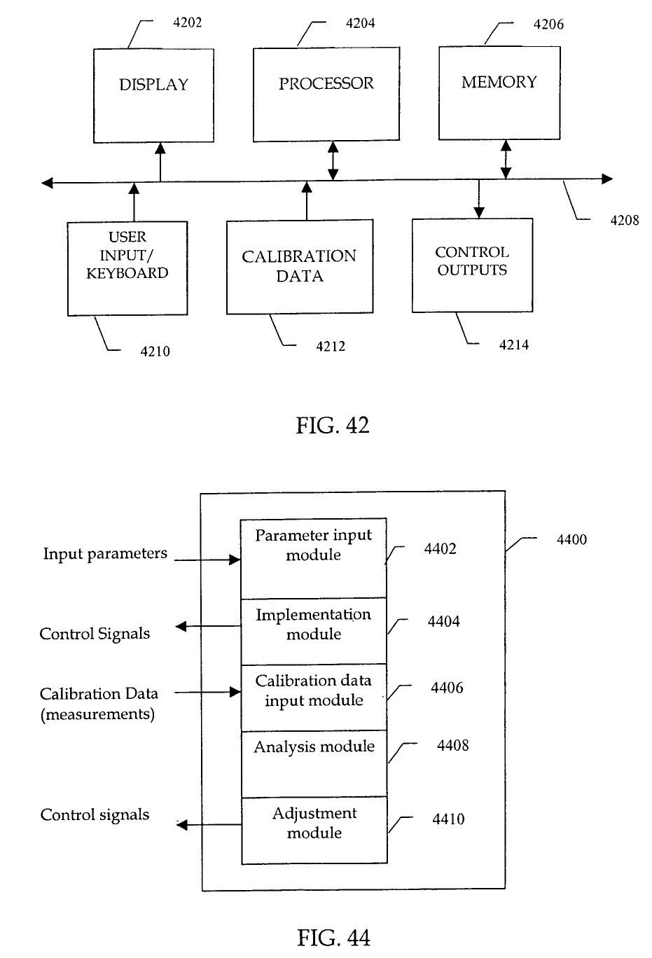

Referring next to FIG. 42, a diagram is shown illustrating the hardware components of a computer based control system implemented for example, within the operating system/user interface 2002 of FIG. 41, in accordance with one embodiment of the invention. The computer-based control system includes a display 4202, a processor 4204, a user input 4210 (for example, keyboard), a memory 4206, calibration data input 4212, control outputs 4214, and a bus 4208. The display 4202, the processor 4204, the user input 4210, the memory 4206, the calibration data input 4212, and the control outputs 4214 are coupled together via the bus 4208.

According to one embodiment, the control system is a computer-based system that inputs settings as desired by the user and automatically implements these settings for the appropriate test or treatment. The system uses feedback from use to initialize and implement the settings, or to fine tune the settings during use, or to maintain the settings throughout the test or treatment. The system operates fully automatically in response to initial user input. Additionally, the system stores all test data and performs analysis of the results.

The software that controls and operates the system is stored in memory 4206 and run or executed by processor 4204. It is understood that processor 4204 may be a single processor, a dual processor or other multiprocessor as is known in the art; however, preferably, the processor is dual processor such that multiple instructions may be executed at the same time by the system. Users input data into the system in response to system prompts via the display 4202 and the user input 4210. Depending upon the embodiment and level of control of the system, the user may enter as initial test variables one or more of the following: flow rate of the fluid product, the number of flashes a particular portion of the product should be exposed to (generally, the exposure time), the viscosity of the fluid product, the density of the fluid product, the media to be treated, the concentration of the media or product, the organism to be deactivated, the inoculation level and the fluence/flash (generally, the dosage). System prompts, as well as user inputs may be displayed for the users review via the display 4202.

In response to the user's inputs, the system translates the input parameters into values usable by the hardware of the light treatment system. For example, the system determines the charge voltage of the light source, the distance from the light source to the product or treatment zone, and the thickness of the treatment zone that will implement the user settings. These values are transmitted to the various system components via the control outputs 4214.

The calibration data input 4212 represents feedback values from the system that are used by the system in order to implement or maintain the system settings or prescribed treatment levels. This data may be any of the data collected or measured in the treatment process as described herein. In some embodiments, this calibration data includes measurements taken during the calibration of the system, e.g., the calibration of a spectrometer device and/or a filter used to attenuate light such as described with reference to FIGS. 26A-27B.

In contrast to known control systems for light treatment devices, all implementation of user inputs is automated, i.e., the user does not make any manual adjustments to implement the settings. Further in contrast to known control systems for light treatment devices, feedback measurements are used to verify and fine tune such settings automatically.

It is noted that the control software may implement the functionality of any of the methods described herein, such as described with reference to FIGS. 15A-39B.

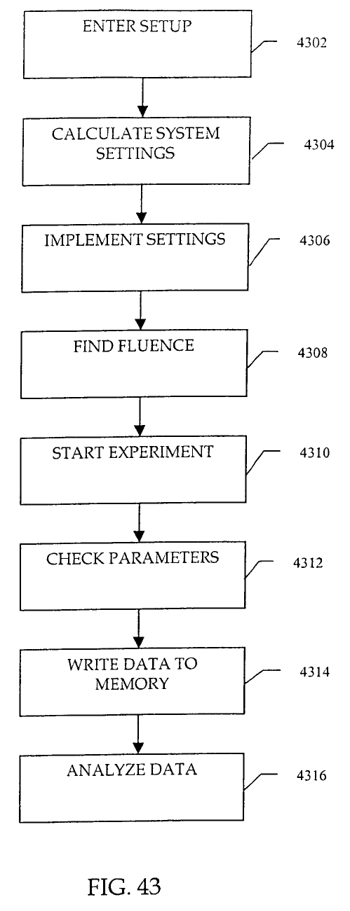

Referring next to FIG. 43, a flowchart is shown of the steps performed by the control software in accordance with one embodiment of the invention. In one embodiment, the functionality of the control software is performed by the control system of FIG. 42, for example, the software is stored in memory 4206 and executed by the processor 4204.

While referring to FIG. 43, concurrent reference will be made to FIG. 44, which illustrates one embodiment of the control software for a computer based control system in terms of functional modules. Illustrated is the control software 4400, a parameter input module 4402, an implementation module 4404, a calibration data input module 4406, an analysis module 4408 and an adjustment module 4410.

Initially, setup is entered (Step 4302). In this step, the software initializes the user interface. For example, the appropriate user interface displays are created to prompt the user for information, such as a password to operate the system. Furthermore, the user is prompted to enter the operating settings desired for the test. These input parameters are input to the parameter input module 4402 of the control software 4400. It is noted that in some embodiments, the input parameters are not from the user, but from another source coupled to the control system or from the control system itself. In one embodiment, the user enters one or more of the following: flow rate, the number of flashes a particular portion of the product should be exposed to, the viscosity of the fluid product, the density of the fluid product, the media to be treated, the concentration of the media or product, the organism to be deactivated, the inoculation level and the fluence/flash. It should be understood that numerous other operating parameters may be input by the user, depending on the configuration and operation of the treatment system.

Next, the system calculates the system settings (Step 4304). According to this step, the system calculates or determines the proper settings for the system to be able to implement the input treatment parameters. For example, if the user input a given fluence level, the system determines voltage to place across the light source and determines the distance from the light source to the treatment zone. Furthermore, the system will determine what value is needed at an actuator or pump device to implement a particular flow rate. For example, the control system calculates the starting position of the light source relative to the product being treated, such as described with reference to FIGS. 35A-37.

Next, these settings are implemented (Step 4306). For example, the system generates control signals which are transmitted to the appropriate components of the light treatment system, such as the pulse generator, light source, actuators, etc. It is noted that Steps 4304 and 4306 may be performed by the implementation module 4404 of the control software 4400 that outputs the appropriate control signals to implement the settings.

Next, the system finds the fluence at the implemented settings (Step 4308). Thus, in one embodiment, the light source is activated, e.g., pulsed, and measurements of the fluence illuminating the treatment zone and transmitting through the treatment zone are taken. In one embodiment, the fluence over multiple wavelengths is measured by a spectrometer. These measurements are used to verify the light treatment parameters. In several embodiments, calibration data is input to the calibration data input module 4406 and analyzed by the analysis module 4408 of the control software 4400. Additionally, in some embodiments, the calibration data input to the calibration data module 4406 includes measurements taken during the calibration of a spectrometer or other calibration, such as the calibration of a filter for attenuation.

Next, an experiment is started (Step 4310). At this point, according to one embodiment, a buffer solution or other solution having a known optical transmission properties is flowed through the treatment chamber or treatment zone and illuminated by the light treatment. For example, WFI from syringe 120 is flowed through the treatment chamber. Again, measurements of the fluence across the multiple wavelengths of the light treatment illuminating the buffer and transmitting through the buffer are measured for each flash of the light treatment and fed back to the control system. These measurements may be used make further adjustments to the light treatment parameters. After the buffer is flowed and the parameters have been verified, the product to be treated is then flowed through the treatment zone and illuminated with at the light treatment, e.g., with at least one pulse of light or with a continuous emission of light energy. Again, in several embodiments, this calibration data or measurements are input to the calibration data input module 4406 and analyzed by the analysis module 4408 of the control software 4400.

Once the product to be treated is flowing, the parameters of the light treatment are again checked (Step 4312). Thus, measurements of the fluence across multiple wavelengths of the light treatment illuminating the product and transmitting through the product are measured. Again, this calibration data is input to the calibration data input module 4406 and analyzed by the analysis module 4408 of the control software 4400. If need be, additional adjustments may be made in response to the measurements to ensure uniform treatment due to changes in the system or changes in properties of the fluid product, such as concentration may be changing. It is also noted that in some embodiments, the preset parameters may dictate changes in system parameters at given points in time.

Any adjustments to the system operating parameter or light treatment parameters are made by the adjustment module 4410 of the control software 4400, which outputs the appropriate control signals.

Next, the operating parameters and settings, as well as all fluence measurements are stored to memory (Step 4314). Then, the stored data set is analyzed (Step 4316), for example, to determine absorption, absorption patterns over time in comparison with other data files. In one embodiment, the analysis is performed by the analysis module 4408 of the control software 4400. It is noted that at each point where fluence measurements are taken by the optical detectors, such as in a spectroradiometer, a curve of the fluence over wavelength is generated. In preferred embodiments, separate curves are generated for the light illuminating the treatment/product and for the light transmitting through the treatment zone/product such that an absorption curve may be generated as the different between the two curves. Such absorption curves are helpful to illustrate how much light at the various wavelengths is absorbed into the fluid product.

Since in some embodiments, the treatment system is an experimental system that is used to determine the optimal operating parameters for a given product, many experiments are performed and a data file is stored for each experiment. This data is then analyzed to determine an optimal set of operating and treatment parameters for a given product.

It is noted that when referring to the functional software modules of FIG. 44, the functionality of different modules may overlap that in other modules. It should be understood in the art that this functionality may be variously placed within different functional modules and submodules and still function equivalently.

Advantageously, as described above, the control software and computer based control system of many embodiments of the invention is fully automated such that an operator does not have to make any manual adjustments to implement treatment runs. Such adjustments are automatically determined and implemented. The control system essentially functions as a technician who will runs tests and make adjustments until the system is ready to operate for a desired treatment or to maintain the system at a desired treatment. Furthermore, the control software 4200 may implement the functionality necessary to carry out one or more of the feedback and control methods as described herein, for example, those described with reference to FIGS. 28-39B.

### EXAMPLES

Next, the following examples are experimental results using a device similar to the fluid treatment system of FIG. 11 to illustrate the response of proteins in blood plasma derivatives to pulsed light, e.g., BSPL treatment emitted from the light source, as well as the deactivation of microorganisms, such as E. coli.

### Example 1

### Protein Damage

Various proteins, such as Alkaline Phosphatase, Lactate Dehydrogenase, acid Phosphatase and Beta Galactoidase were tested for their susceptibility to BSPL. Each protein was contained within fluid at a total protein concentration of 5 mg/ml and treated in a static chamber. Treatments were formed in 1 ml samples, in replicates of three. Each sample was subjected to N 0.25 J flashes of BSPL, where N=1, 2, 4, 6, 8, and 12. This corresponds to a total energy of 0.25 J, 0.5 J, 1.0 J, 1.5 J, 2.0 J and 3.0 J, respectively for N=1, 2, 4, 6, 8 and 12. Following the treatment, each protein or enzyme was assayed to determine the percent of enzyme activity remaining. The result is plotted in FIG. 45 as a % of protein activity remaining vs. the number of flashes.

As seen in FIG. 45, different proteins (enzymes) are susceptible to BSPL to differing extents. Line 2102 corresponds to Alkaline phosphate, line 2104 corresponds to Lactate Dehydrogenate, line 2106 corresponds to Acid Phosphatase, and line 2108 corresponds to Beta galactosidase. Alkaline phosphatase is very resistant to BSPL showing no loss of protein activity even with 3 joules of total energy, whereas beta-galactosidase is far less resistant showing activity loss with as little as 0.25 joules of total BSPL energy. Thus, since it is desired to deactivate microorganisms (such as viruses, bacteria, etc.) within the bioprocessing fluids with minimal protein damage, the fluence of the light treatment and the number flashes that portions of the fluid are subjected to will vary greatly depending on the specific proteins present in the bioprocessing fluid.

### Example 2

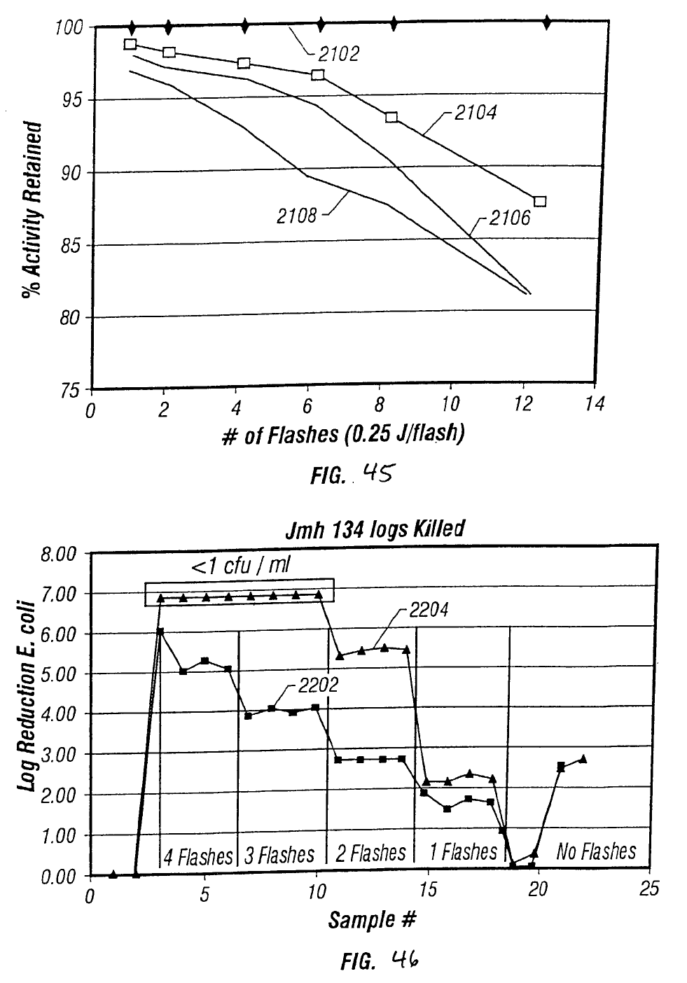

Example 2 involves the use of the “Staircase” test to determine treatment kinetics and system response in-flow of the fluid treatment system of FIG. 10 with fluid containing 5 mg/ml bovine serum albumin (BSA). BSA is a form of serum albumin that is a known protein that is effective in protecting other molecules from degradation due to BSPL. BSA is a readily available source of serum albumin, which is commonly used in in vitro biological studies, as a replacement for human albumin. The samples were pumped at a flow rate of 250 ml/min. The experiment provides a high initial treatment level, gradually decreasing to no treatment over the course of a 20-minute test run. The flash rate of the pulse generator coupled to the flashlamp is synchronized with the fluid flow rate to provide an effective treatment of 4, 3, 2, 1 and 0 pulses. The sample rate is 1 sample per minute. The fluid sample was also inoculated with E. coli. The results are plotted in FIG. 46 for two different treatment levels, a “low” fluence of 0.1 J/cm² per flash and a “high” fluence of 0.2 J/cm² per flash. As seen in FIG. 46, line 2202 represents the low fluence while line 2204 represents the high fluence. This data shows how parameters such as flash rate, flow rate, number of flashes and fluence per flash can be tuned to provide the desired level of microbial kill and/or product activity recovery.

### Example 3

Based on optimization tests performed, such as in EXAMPLE 2, an optimum operating point was selected to provide a desired kill level of E. coli and operated for approximately two hours. In this example, the protein concentration (BSA) was 5 mg/ml BSA and the flow rate was 300 ml/min. The fluid also contained E. coli and Beta galactosidase. The treatment level was 0.5 J/cm² per flash and the total energy was between 1.5 and 3 J/cm². Samples were taken every 4 minutes in the extended run and the results of the log reduction of E. coli vs. the time the sample are plotted in FIG. 47. As can bee seen in FIG. 47, over the two-hour period, the level of kill was between 6 and 7 logs reduction, i.e., a most desirable range for microorganism inactivation and a commonly accepted level of sterilization for many applications. It is noted that an alternate pump assembly and fluid container was used to allow the test fluid to be pumped continuously for two hours (as opposed to the syringes described above). Thus, as can be seen, BSPL is very effective in deactivating microorganisms, even while operated under parameters to minimize protein damage in bioprocessing fluids, such as blood plasma derivatives.

### Example 4

### Treatment Depth 3 mm

In EXAMPLES 4-6, tests were performed to test both kill (in these experiments E. coli) and protein activity degradation (in this case Beta galactosidase or Beta gal.) as experimental outputs. In many embodiments, it is a goal to achieve a high level of kill to a low level of protein activity degradation or protein damage. Thus, a useful metric for an indication of treatment efficacy and as a tool for treatment optimization is the ratio of protein damage (in % activity reduction) to the kill level (in logs reduction). A lower damage/kill ratio is better. For example, 5 logs of kill with 30% Beta-gal. damage provides a damage/kill ratio=6. Five logs kill with 25% damage provides a better damage/kill=5.

In EXAMPLE 4, Bovine serum albumin (BSA) (Sigma 40K0898) was reconstituted to concentrations of (5, 10, 15, 25 and 50) mg/ml, mixed with 3 mg/ml Beta-galactosidase (ICN 7026B) at a 1 to 1000 dilution (Beta-galactosidase activity is used to monitor protein damage) and inoculated with E. coli (ATCC 11775) to 10⁶ cfu/ml. Each inoculated concentration was pumped through a 1/11th-laboratory scale treatment chamber (e.g., treatment chamber 702) at a flow rate of 200 ml/min with a treatment depth of 3 mm (as adjusted by altering the distance between the respective window plates of a cartridge). As the concentrations of the fluid passed through the treatment chamber each was exposed to broad spectrum pulsed light from a single flashlamp positioned to deliver energy levels between 0.1 J/cm² and 0.68 J/cm² per flash. The flash frequency was varied based on the center line velocity such that the fluid passing through the center of the treatment zone received between 1 and 5 exposures. Samples of treated concentrations were collected and assayed for Beta-galactosidase activity and E. coli kill.

The results of these tests are shown in TABLE 1. The number in TABLE 1 is the protein damage to kill ratio and the number in parenthesis is the number of flashes needed. E. coli kill was found to be BSA concentration dependent through all energy levels tested. Greater than 6 logs of kill was achieved at fluence or energy levels of (0.2, 0.3, 0.4, and 0.68) J/cm2 per flash for differing concentrations of BSA. The respective concentration and number of flashes at each of these flowing conditions was (4 flashes at 0.2 J/flash for 5 mg/ml BSA), (5 flashes at 0.3 J/flash for 10 mg/ml BSA), (3 flashes at 0.4 J/flash for 10 mg/ml BSA) and (4 flashes at 0.68 J/flash for 15 mg/ml BSA). Protein damage measured as a function of Beta-galactosidase activity for each of the above flowing conditions was less than 30% in all cases. This corresponds to damage/kill ratios of 6 or less. For example, in some cases, the damage to kill ratio is less than 5, less than 4, less than 3, and less than 2.

TABLE 1
Protein Damage/Kill ratio (Treatment Depth 3 mm)

| [BSA] | | | | | |
|---|---|---|---|---|---|
| (mg/ml) | 0.1 J/flash | 0.2 J/flash | 0.3 J/flash | 0.4 J/flash | 0.68 J/flash |
| 0 | 1.0 (2)* | 5.0 (1) | 6.0 (1) | 9.0 (1) | 15.8 (1) |
| 5 | | 4.2 (4) | 2.9 (4)* | 3.8 (3) | 6.1 (2) |
| 10 | | | 5.0 (9)** | 3.3 (3-4)* | 4.4 (3) |
| 15 | | | | 5.0 (8)** | 4.3 (4-5)* |
| 25 | | | | | 9.0 (15)** |
| 50 | | | | | >12 (25)** |

*optimum test condition for a given concentration of BSA
**extrapolated value

### Example 5

### Treatment Depth 1 mm

In this example, Bovine serum albumin (BSA) (Sigma 40K0898) was reconstituted to concentrations of (5, 10, 15, 25 and 50) mg/ml, mixed with 3 mg/ml Beta-galactosidase (ICN 7026B) at a 1 to 1000 dilution (Beta-galactosidase activity is used to monitor protein damage) and inoculated with E. coli (ATCC 11775) to 10⁶ cfu/ml. Each inoculated concentration was pumped through a 1/11th-laboratory scale treatment chamber (e.g., treatment chamber 702) at a flow rate of 61 ml/min with a treatment depth of 1 mm. As the concentrations passed through the treatment chamber each was exposed to board spectrum pulsed light from a single lamp positioned to deliver fluence or energy levels between 0.1 J/cm² and 0.3 J/cm² per flash. The flash frequency was varied based on the center line velocity such that the fluid passing through the center of the treatment zone received between 1 and 5 exposures. Samples of treated concentrations were collected and assayed for Beta-galactosidase activity and E. coli kill.

The results of these tests are shown in TABLE 2. Again, E. coli kill was found to be BSA concentration dependent through all energy levels tested. Greater than 5 logs of kill was achieved at different fluence or energy levels (of 0.1, 0.2 and 0.3 J/cm² per flash) at differing concentrations of BSA. Respective concentration and number of flashes at each of these flowing conditions was (4 flashes at 0.1 J/flash for 25 mg/ml BSA), (3 flashes at 0.2 J/flash for 25 mg/ml BSA) and (5 flashes at 0.3 J/flash for 50 mg/ml BSA). Protein damage measured as a function of Beta-galactosidase activity for each of the above flowing conditions was less than 25% in all cases. Thus, as can be seen damage/kill ratios of less than 5, less than 6, less than 4, and less than 3 are achievable, respectively.

TABLE 2
Protein Damage/Kill ratio (Treatment Depth 1 mm)

| [BSA] (mg/ml) | 0.1 J/flash | 0.2 J/flash | 0.3 J/flash |
|---|---|---|---|
| 0 | 12.8 (1) | 13.6 (1) | |
| 5 | 6.0 (2-3) | 6.7 (2) | |
| 10 | 2.5 (3-4)* | 5.2 (2-3) | |
| 15 | 3.1 (4-5)* | | |
| 25 | 3.6 (5-6)* | 4.0 (4) | 3.9 (4) |
| 50 | | | 4.4 (5-6)* |

*optimum test condition for a given concentration of BSA

### Example 6

### Treatment Depth 0.2 mm

In this example, Bovine serum albumin (BSA) (Sigma 40K0898) was reconstituted to concentration of 100 mg/ml, mixed with 3 mg/ml Beta-galactosidase (ICN 7026B) at a 1 to 1000 dilution (Beta-galactosidase activity is used to monitor protein damage) and inoculated with E. coli (ATCC 11775) to 10⁶ cfu/ml. The inoculated concentration was pumped through a 1/11th-laboratory scale treatment chamber at a flow rate of 20 ml/min with a treatment depth of 0.2 mm. As the solution passed through the treatment chamber it was exposed to board spectrum pulsed light from a single lamp positioned to deliver a fluence or energy level of 0.1 J/cm² per flash. The flash frequency was varied based on the center line velocity such that the fluid passing through the center of the treatment zone received between 1 and 5 exposures. Samples of treated concentrations were collected and assayed for Beta-galactosidase activity and E. coli kill.

A protein damage/kill ratio of 5.2 was obtained with a 100 mg/ml concentration of BSA treated with 0.1 J/flash, and yielding greater than 1.5 logs of kill (e.g., 1.7). The respective number of flashes at this flowing condition was 4 flashes. Protein damage measured as a function of Beta-galactosidase activity for the above flowing condition was less than 10%.

### Example 7

### Spectral Profile

In this example, and referring to FIG. 48, an illustration is shown of the output of spectral irradiance monitoring instrument (SIMI), e.g., one embodiment of the process controllers described herein (e.g., process controller 1512, 1628, 1706, 2620) in monitoring light transmitted through the treatment chamber during a product run. Water (one example of a suitable buffer fluid) is initially pumped through the treatment chamber to establish flow within the system and provide baseline diagnostic data. The curve 2402 shows a typical spectral radiant energy measurement when water is flowing through the treatment chamber, compared to the spectral radiant energy measured through a protein solution product as shown in curve 2404. Note that the measurements are nearly identical for wavelengths above 400 nm. This sample protein solution absorbs significantly below 400 nm, causing significantly lower UV energy measurement compared to the water. The ratio of the two measurements as well as the spectral signature of the protein solution can be very useful in analyzing the characteristics of the protein solution and the parameters of the treatment. It is noted that the difference between the two curves 2402 and 2404 at a given wavelength represents the amount of radiant energy absorbed by the protein solution at the given wavelength. Thus, an absorption profile across a spectrum of wavelengths may be generated by taking the difference between curves 2402 and 2404 for each wavelength within the spectrum of wavelengths of interest. For example, such an absorption profile is illustrated in FIG. 17.

### Example 8

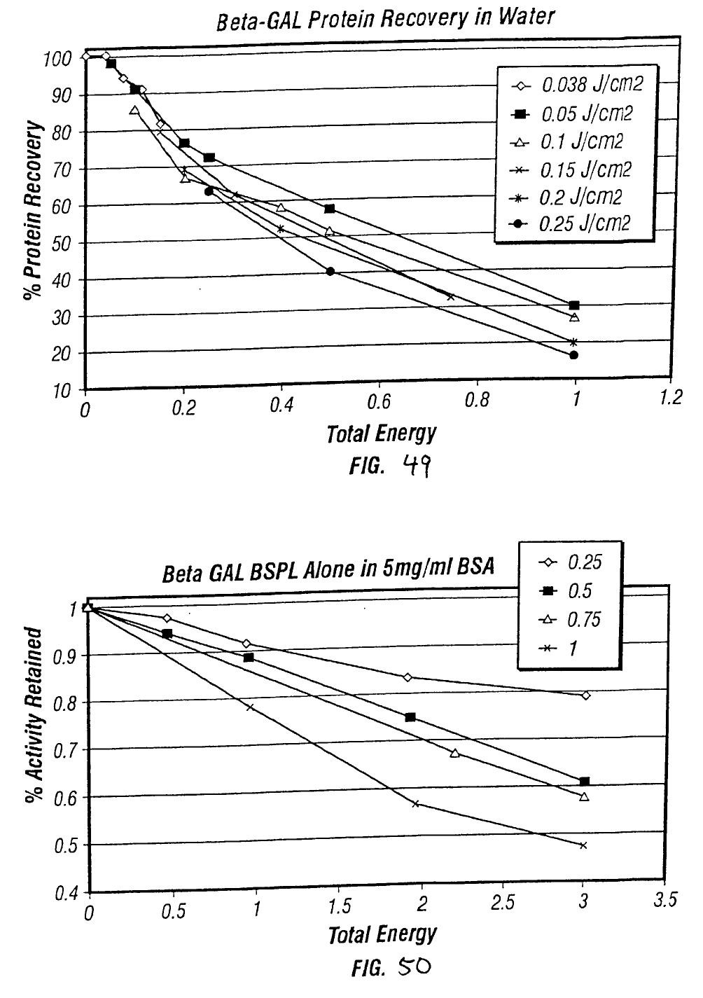

As illustrated in FIG. 49, a graph is shown of percentage of protein recovery vs. the total energy of BSPL for various fluence levels/flash. In this case, the protein tested was Beta-galactosidase within water at flashes of 0.038, 0.05, 0.1, 0.15, 0.2, and 0.25 J/flash. It can be seen that generally at lower fluence levels, such as 0.038 J/cm² and 0.05 J/cm², more protein activity of the Beta-gal. remains after light treatment.

### Example 9

As illustrated in FIG. 50, the percentage of protein activity remaining of Beta-gal within 5 mg/ml BSA vs the total energy of light illuminating the solution is illustrated. The solution was tested with flash fluence or intensities of 0.25, 0.5, 0.75 and 1 J/cm². Again, as seen, at lower fluence levels, such as 0.25 and 0.5 J/cm², the percentage of remaining protein activity is highest.

Other examples and test results involving the illumination of biological fluids, such as blood plasma derivatives with pulsed polychromatic light, such as BSPL, are provided variously in the following co-pending patent applications, each of which is incorporated herein in its entirety by reference: U.S. application Ser. No. 09/329,018, to Cover et al., filed Jun. 9, 1999, entitled METHODS OF INACTIVATING VIRUSES, BACTERIA AND OTHER PATHOGENS, IN BIOLOGICALLY DERIVED COMPOSITIONS, USING BROAD-SPECTRUM PULSED LIGHT; U.S. application Ser. No. 09/502,190, to Cover et al., filed Feb. 11, 2000, entitled PROTECTING MOLECULES IN BIOLOGICALLY DERIVED COMPOSITIONS WHILE TREATING WITH BROAD-SPECTRUM PULSED LIGHT; and U.S. application Ser. No. 09/596,987, to Holloway et al., filed Jun. 20, 2000, entitled THE INACTIVATION OF NUCLEIC ACIDS USING BROAD-SPECTRUM PULSED LIGHT, all of which are incorporated herein by reference.

While the invention herein disclosed has been described by means of specific embodiments and applications thereof, numerous modifications and variations could be made thereto by those skilled in the art without departing from the scope of the invention set forth in the claims.

What is claimed is:

1. A method for use with a treatment system using light comprising:
illuminating a product with a light treatment comprising light having a spectrum of wavelengths within a range of 170 to 2600 nm, the light treatment for treating the product; and
measuring a fluence of a portion of the light treatment for each of a plurality of wavelengths of the spectrum of wavelengths simultaneously.

2. The method of claim 1 wherein the measuring comprises:
measuring the fluence of a portion of the light treatment illuminating the product for each of the plurality of wavelengths of the spectrum of wavelengths simultaneously.

3. The method of claim 1 wherein the product is transmissive to at least 1% of the light having the plurality of wavelengths, wherein the measuring comprises:
measuring the fluence of a portion of the light treatment transmitting through the product for each of the plurality of wavelengths of the spectrum of wavelengths simultaneously.

4. The method of claim 1 further comprising:
collecting the portion of the light treatment at a single optical collector, the measuring step comprising measuring the fluence of the portion of the light treatment having been collected.

5. The method of claim 1 wherein the illuminating comprises:
illuminating the product with the light treatment, the light treatment comprising at least one pulse of light.

6. The method of claim 3 wherein the measuring step comprises:
measuring the fluence of a portion of each pulse of light for each of the plurality of wavelengths of the spectrum of wavelengths simultaneously for each pulse of light.

7. The method of claim 1 wherein the measuring step comprises measuring the fluence using a spectroradiometer.

8. The method of claim 7 wherein the spectrometer comprises a spectroradiometer.

9. The method of claim 1 wherein the measuring comprises measuring a fluence level of the portion of the light treatment for each of the plurality of wavelengths of the spectrum of wavelengths simultaneously.

10. The method of claim 1 wherein the light treatment is for the deactivation of microorganisms.

11. The method of claim 1 wherein the light treatment is for the modification of the product.

12. The method of claim 1 wherein the product comprises a fluid product, the method further comprising:
flowing the fluid product through a treatment chamber of a fluid flow path positioned to receive the light treatment;
the illuminating step comprising illuminating the fluid product as it flows through the treatment chamber with the light treatment.

13. A treatment system using light comprising:
a light source for providing a light treatment, the light treatment having a spectrum of wavelengths within a range of 170 to 2600 nm;
a treatment chamber containing a product to be treated with the light treatment, the light treatment for treating the product; and
a spectrometer having an input collector positioned to receive a portion of the light treatment, the spectrometer for measuring a fluence of the portion of the light treatment for each of a plurality of wavelengths of the spectrum of wavelengths simultaneously.

14. The system of claim 9 wherein the spectrometer measures the fluence of a portion of the light treatment illuminating the product for each of the plurality of wavelengths of the spectrum of wavelengths simultaneously.

15. The system of claim 13 wherein the product is transmissive to at least 1% of the light having the plurality of wavelengths, wherein the spectrometer measures the fluence of a portion of the light treatment transmitting through the product for each of the plurality of wavelengths of the spectrum of wavelengths simultaneously.

16. The system of claim 13 wherein the light source comprises a pulsed light source for providing at least one pulse of light; and
wherein the spectrometer measures the fluence of a portion of each pulse of light for each of the plurality of wavelengths of the spectrum of wavelengths simultaneously for each pulse of light.

17. The system of claim 13 wherein the spectrometer comprises a spectroradiometer for measuring a fluence level of the portion of the light treatment for each of the plurality of wavelengths of the spectrum of wavelengths simultaneously.

18. The system of claim 13 wherein the light treatment is for the deactivation of microorganisms.

19. A method for use with a system for the deactivation of microorganisms using light comprising:
illuminating a product with a light treatment having a spectrum of wavelengths, the product being transmissive to at least 1% of light having at least one wavelength within a range of 170 to 2600 nm, the light treatment intended to treat the product;
measuring a fluence level for a portion of the light treatment illuminating the product for each of a plurality of wavelengths of the spectrum of wavelengths;
measuring a fluence level for a portion of the light treatment transmitting through the product for each of the plurality of wavelengths of the spectrum of wavelengths; and
generating an absorption profile across each of the plurality of wavelengths for the product based upon a comparison of the results of the measuring steps.

20. The method of claim 19 further comprising:
identifying an absorption peak at a respective one of the plurality of wavelengths of the spectrum of wavelengths for the product.

21. The method of claim 19 further comprising:
comparing the absorption profile to a known valid absorption profile for the product illuminated with the light treatment;
verifying that the absorption profile correlates to the known valid absorption profile.

22. The method of claim 21 further comprising:
identifying a deviation of the absorption profile in comparison to the known valid absorption profile.

23. The method of claim 22 wherein the identifying comprises:
identifying a deviation of the absorption profile in comparison to the known valid absorption profile at at least one wavelength within the plurality of wavelengths.

24. The method of claim 19 further comprising determining an amount of energy absorbed into the product.

25. The method of claim 19 wherein the light treatment comprises a pulse of light, the determining the amount of energy absorbed step comprising:
determining the amount of energy absorbed into the product for the pulse of light.

26. The method of claim 19 further comprising:
measuring, at a subsequent point in time, the fluence level for the portion of the light treatment illuminating the product for each of the plurality of wavelengths of the spectrum of wavelengths;
measuring, at the subsequent point in time, the fluence level for the portion of the light treatment transmitting through the product for each of the plurality of wavelengths of the spectrum of wavelengths;
generating another absorption profile across each of the plurality of wavelengths for the product based upon a comparison of the results of the measuring at the subsequent point in time steps, the other absorption profile corresponding to the subsequent point in time; and
comparing the absorption profile and the other absorption profile to determine if a change in the absorption has occurred.

27. The method of claim 26 further comprising:
determining if a change in absorption has occurred at one or more selected wavelengths of the plurality of wavelengths; and
setting an operating condition of the system based upon a degree of change in absorption at the one or more selected wavelengths.

28. The method of claim 27 wherein the operating condition is selected from a group of operating condition comprises a pass condition or a fail condition.

29. The method of claim 19 wherein the illuminating comprises illuminating the product with the light treatment, the light treatment comprising at least one pulse of light.

30. The method of claim 29 wherein the generating the absorption profile comprises determining the absorption profile on a per pulse basis.

31. The method of claim 30 wherein the measuring steps comprise:
measuring the respective fluence levels for each of the plurality of wavelengths of the spectrum of wavelengths for each pulse of light.

32. The method of claim 19 wherein the measuring steps comprise measuring the respective fluence levels using a spectroradiometer.

33. The method of claim 19 wherein the product comprises a fluid product, the method further comprising:
flowing the fluid product through a treatment chamber positioned to receive the light treatment;
the illuminating step comprising illuminating the fluid product with the light treatment during the flowing step.

34. A monitoring system for use with a treatment system for treating products using light comprising:
a light source for illuminating a product with a light treatment having a spectrum of wavelengths, the product being transmissive to at least 1% of light having at least one wavelength within a range of 170 to 2600 nm, the light treatment intended to treat the product;
a first optical detector positioned to measure a fluence level for a portion of the light treatment illuminating the product for each of a plurality of wavelengths of the spectrum of wavelengths;
a second optical detector positioned to measure a fluence level for a portion of the light treatment transmitting through the product for each of the plurality of wavelengths of the spectrum of wavelengths; and
a controller coupled to the first optical detector and the second optical detector for generating an absorption profile across the plurality of wavelengths for the product based upon a comparison of the results of the measuring steps.

35. The system of claim 34 wherein the controller is adapted to identify an absorption peak at a respective one of the plurality of wavelengths of the spectrum of wavelengths for the product.

36. The system of claim 34 wherein the controller is adapted to perform the following steps:
comparing the absorption profile to a known valid absorption profile for the product illuminated with the light treatment; and
verifying that the absorption profile correlates to the known valid absorption profile.

37. The system of claim 36 wherein the controller is adapted to identify a deviation of the absorption profile in comparison to the known valid absorption profile.

38. The system of claim 34, the controller adapted to perform the following additional steps:
receiving measurements, at a subsequent point in time, of the fluence level for the portion of the light treatment illuminating the product for each of the plurality of wavelengths of the spectrum of wavelengths;
receiving measurements, at the subsequent point in time, of the fluence level for the portion of the light treatment transmitting through the product for each of the plurality of wavelengths of the spectrum of wavelengths;
generating another absorption profile across the plurality of wavelengths for the product based upon a comparison of the received measurements at the subsequent point in time steps, the other absorption profile corresponding to the subsequent point in time; and
comparing the absorption profile and the other absorption profile to determine if a change in the absorption has occurred.

39. The system of claim 34 further comprising a spectroradiometer coupling the first and second optical detectors to the controller, the first and second optical detectors comprising optical collectors for the spectroradiometer.

40. A method for use with a treatment system using light comprising:
illuminating a treatment chamber with a light treatment having a spectrum of wavelengths, the treatment chamber transmissive to at least 1% of light having at least one wavelength within a range of 170 to 2600 nm, the treatment chamber being empty but adapted to flow a product therethrough that is to be treated with the light treatment;
measuring a fluence level for a portion of the light treatment illuminating the treatment chamber for each of a plurality of wavelengths of the spectrum of wavelengths;
measuring a fluence level for a portion of the light treatment transmitting through the treatment chamber for each of the plurality of wavelengths of the spectrum of wavelengths;
comparing the respective fluence levels measured for each of the plurality of wavelengths; and
determining, based upon the comparing step, whether the treatment chamber is ready for the product to be flowed through the treatment chamber for operation.

41. The method of claim 40 wherein the determining step comprises:
determining, based upon the comparing step, whether optical absorption of the treatment chamber at the plurality of wavelengths is within an acceptable operating range.

42. The method of claim 40 wherein the illuminating step comprises illuminating the treatment chamber with the light treatment, the light treatment comprising at least one pulse of light.

43. The method of claim 42 wherein the measuring steps comprise:
measuring the respective fluence levels for each of the plurality of wavelengths for each pulse of light.

44. The method of claim 40 wherein the measuring steps comprise measuring the respective fluence levels using a spectroradiometer.

45. A monitoring system for use with a treatment system using light comprising:
a light source for illuminating a treatment chamber with a light treatment having a spectrum of wavelengths, the light treatment having a known fluence level at each of a plurality of wavelengths of the spectrum of wavelengths;
the treatment chamber transmissive to at least 1% of light having at least one wavelength within a range of 170 to 2600 nm, the treatment chamber being empty but adapted to flow a product therethrough that is to be treated with the light treatment;
a first optical detector for measuring a fluence level for a portion of the light treatment illuminating the treatment chamber for each of the plurality of wavelengths of the spectrum of wavelengths;
a second optical detector for measuring a fluence level for a portion of the light treatment transmitting through the treatment chamber for each of the plurality of wavelengths of the spectrum of wavelengths; and
a controller coupled to the first optical detector and the second optical detector, the controller adapted to perform the following steps:
comparing the respective fluence levels measured for each of the plurality of wavelengths; and
determining, based upon the comparing step, whether the treatment chamber is ready for the product to be flowed through the treatment chamber for operation.

46. The system of claim 45 wherein the light source comprises a pulsed light source.

47. The system of claim 45 further comprising a spectroradiometer coupling the first and second optical detectors to the controller, the first and second optical detectors comprising optical collectors for the spectroradiometer.

48. A method for use with a treatment system using light comprising:
flowing a buffer fluid through a fluid flow path of the treatment system, the buffer fluid having known physical and optical absorption properties across a plurality of wavelengths of a spectrum of wavelengths;
illuminating the buffer fluid with a light treatment having a known fluence level at each of the plurality of wavelengths of the spectrum of wavelengths, a portion of the fluid flow path and the product are transmissive to at least 1% of light having at least one wavelength within a range of 170 to 2600 nm;
measuring a fluence level at one or more of the plurality of wavelengths for a portion of the light treatment transmitting through the buffer fluid;
verifying, based on the measuring step, the optical absorption properties of the buffer fluid;
determining, based upon the verifying step, whether the optical properties of the fluid flow path are within an acceptable range for operation.

49. The method of claim 48 further comprising:
flowing, after the determining step, a fluid product through the fluid flow path, the fluid product to be treated with the light treatment; and
illuminating the fluid product with the light treatment.

50. The method of claim 48 further comprising:
measuring a fluence level at one or more of the plurality of wavelengths for a portion of the light treatment illuminating the buffer fluid;
comparing the fluence level having been measured for the portion of the light treatment illuminating the buffer fluid with a known fluence level for each of the one or more wavelengths of the light treatment.

51. The method of claim 50 further comprising:
verifying, based on the comparing step, the preset fluence level for each of the one or more wavelengths of the light treatment.

52. The method of claim 48 wherein the illuminating comprises illuminating the buffer fluid with the light treatment, the light treatment comprising at least one pulse of light.

53. The method of claim 52 wherein the measuring step comprises:
measuring the fluence level at one or more of the plurality of wavelengths for the portion of the light treatment transmitting through the buffer fluid for each pulse of light illuminating the buffer fluid.

54. The method of claim 48 wherein the measuring step comprises measuring the fluence level using a spectroradiometer.

55. A monitoring system for use with a treatment system using light comprising:
a fluid flow path of the treatment system for flowing a buffer fluid therethrough, the buffer fluid having known physical and optical absorption properties across a plurality of wavelengths of a spectrum of wavelengths;
a light source for illuminating the buffer fluid with a light treatment having a known fluence level at each of the plurality of wavelengths of the spectrum of wavelengths, wherein a portion of the fluid flow path and the product are transmissive to at least 1% of light having at least one wavelength within a range of 170 to 2600 nm;
an optical detector positioned to measure a fluence level at one or more of the plurality of wavelengths for a portion of the light treatment transmitting through the buffer fluid; and
a controller coupled to the optical detector, the controller adapted to perform the following steps:
verifying, based on the measuring step, the optical absorption properties of the buffer fluid; and
determining, based upon the verifying step, whether the optical properties of the fluid flow path are within an acceptable range for operation.

56. A method for use with a treatment system using light comprising:
flowing a buffer fluid through a fluid flow path of the treatment system, the buffer fluid having known physical and optical absorption properties, the flowing establishing an operational condition of the treatment system;
determining whether the operational condition has been established;
flowing a fluid product through the fluid flow path, the fluid product to be treated with a light treatment; and
illuminating the fluid product with the light treatment.

57. The method of claim 56 wherein the flowing the buffer fluid establishes a flow geometry of a portion of the fluid flow path.

58. The method of claim 57 wherein the determining step comprises:
measuring a flow pressure of the fluid flow path; and
verifying that the flow pressure having been measured is within an acceptable range.

59. The method of claim 56 wherein the flowing the buffer fluid establishes a flow rate of the buffer fluid through a portion of the fluid flow path.

60. The method of claim 59 wherein the determining step comprises:
measuring a flow rate of the buffer fluid through the portion of the fluid flow path; and
verifying that the flow rate having been measured is substantially equal to a preset flow rate.

61. The method of claim 56 wherein the illuminating comprises illuminating the buffer fluid with the light treatment, the light treatment comprising at least one pulse of light.

62. A treatment system using light comprising:
a fluid flow path of the treatment system for flowing a buffer fluid therethrough to establish an operational condition of the treatment system, the buffer fluid having known physical and optical absorption properties;
means for determining whether the operational condition has been established;
means for flowing a fluid product through the fluid flow path, the fluid product to be treated with the light treatment; and
a light source for illuminating the fluid product with a light treatment.

63. A method for use with a fluid treatment system using light comprising:
illuminating a treatment chamber of a treatment system with a light treatment, the treatment chamber containing a product to be treated with the light treatment, a portion of the treatment chamber and the product transmissive to at least 1% of light having at least one wavelength within a range of 170 to 2600 nm;
measuring a fluence level of a portion of the light treatment transmitting through the treatment chamber at a first location proximate to a first portion of the treatment chamber; and
measuring a fluence level of a portion of the light treatment transmitting through the treatment chamber at a second location proximate to a second portion of the treatment chamber, the second location positionally offset from the first location, the first location and the second location within a portion of a profile of the treatment chamber.

64. The method of claim 63 wherein the illuminating step comprises:
illuminating the treatment chamber with the light treatment, the light treatment comprising at least one pulse of light.

65. The method of claim 63 further comprising:
flowing a fluid through the treatment chamber; and
comparing the measured fluence measurements.

66. The method of claim 65 further comprising:
measuring a fluence level of a portion of the light treatment illuminating the treatment chamber proximate to the first location of the treatment chamber;
measuring a fluence level of a portion of the light treatment illuminating the treatment chamber proximate to the second location of the treatment chamber.

67. The method of claim 66 wherein the comparing step comprises:
determining a first absorption level at the first portion of the treatment chamber as a difference between the measured fluence level of the portion of the light treatment illuminating the treatment chamber proximate to the first location and the measured fluence level of the portion of the light treatment transmitting through the treatment chamber at the first location;
determining a second absorption level at the second portion of the treatment chamber as a difference between the measured fluence level of the portion of the light treatment illuminating the treatment chamber proximate to the second location and the measured fluence level of the portion of the light treatment transmitting through the treatment chamber at the second location; and
comparing the first absorption level and the second absorption level.

68. The method of claim 65 wherein the first portion comprises an entrance portion of the treatment chamber and the second portion comprises an exit portion of the treatment chamber.

69. The method of claim 65 further comprising:
determining, based on the comparing step, a change in a property of the fluid from the first portion across a length of fluid flow to the second portion of the treatment chamber.

70. The method of claim 69 wherein the property comprises a change in concentration of a contaminant within the fluid.

71. The method of claim 69 wherein the fluid comprises a protein solution, wherein the property comprises a change in concentration of protein within the fluid.

72. The method of claim 65 further comprising determining, based on the comparing step, a change in a geometry of the treatment chamber.

73. The method of claim 65 further comprising determining, based on the comparing step, a buildup of denatured material within the treatment chamber.

74. The method of claim 65 wherein the fluid comprises a fluid product to be treated with the light treatment.

75. The method of claim 63 creating a dose mapping of at least a portion of the profile of the treatment chamber based upon the measuring steps.

76. The method of claim 75 wherein the product comprises a fluid product, the method further comprising:
flowing the fluid product through the treatment chamber while illuminating the treatment chamber and the fluid product.

77. The method of claim 75 further comprising:
measuring the fluence level of a portion of the light treatment transmitting through the treatment chamber at a plurality of additional locations proximate to additional portions of the treatment chamber, each additional location positionally offset from each other and the first location and the second location.

78. The method of claim 77 wherein the first location, the second location and the additional locations substantially cover at least the portion of the profile of the treatment chamber.

79. The method of claim 77 wherein the measuring steps occur at substantially the same time.

80. The method of claim 77 wherein measuring steps comprise:
measuring the fluence levels using a plurality of optical detectors, the plurality of optical detectors arranged at separate locations across the dimensions of the at least the portion of profile of the treatment chamber.

81. The method of claim 80 wherein the plurality of optical detectors are arranged on a detector array.

82. The method of claim 75 further comprising:
positioning an optical detector at the first location prior to the illuminating;
the illuminating step comprising:
illuminating the treatment chamber and the product with a first light treatment;
repositioning the optical detector to the second location after the illuminating with the first light treatment; and
the illuminating step further comprising:
illuminating the treatment chamber and the product with a second light treatment.

83. The method of claim 82 wherein the measuring the fluence level at the first location comprises:
measuring the fluence level of the portion of the first light treatment transmitting through the treatment chamber at the first location; and
wherein the measuring the fluence level at the second location comprises:
measuring the fluence level of the portion of the second light treatment transmitting through the treatment chamber at the second location.

84. The method of claim 82 wherein the product comprises a fluid product, the method further comprising:
flowing the fluid product through the treatment chamber while illuminating the treatment chamber and the fluid product.

85. A light treatment monitoring system comprising:
a treatment chamber for containing a product to be treated with a light treatment, at least a portion of the treatment chamber and the product transmissive to at least 1% of light having at least one wavelength within a range of 170 to 2600 nm;
a first optical detector positioned to measure a fluence level of light transmitting through a first portion of the treatment chamber; and
a second optical detector positioned to measure a fluence level of light transmitting through a second portion of the treatment chamber, the second portion positionally offset from the first location.

86. The system of claim 85 further comprising a light source for providing the light treatment.

87. The system of claim 86 wherein the light source comprises a pulsed light source.

88. The system of claim 85 wherein the treatment chamber is adapted to flow a fluid therethrough, the second portion located at a postion further along a length of fluid flow within the treatment chamber.

89. The system of claim 88 further comprising a controller coupled to the first optical detector and the second optical detector, the controller for comparing the measured fluence levels to determine changes along the length of the fluid flow from the first location to the second location.

90. The system of claim 89 further comprising:
a third optical detector coupled to the controller and positioned to measure a fluence level of light illuminating the first portion of the treatment chamber; and
a fourth optical detector coupled to the controller and positioned to measure a fluence level of light illuminating the second portion of the treatment chamber.

91. The system of claim 89 wherein the first portion comprises an entrance portion of the treatment chamber and the second portion comprises an exit portion of the treatment chamber.

92. The system of claim 88 wherein the fluid comprises a fluid product to be treated with the light treatment.

93. The system of claim 85 further comprising a controller coupled to the first optical detector and the second optical detector for creating a dose mapping of at least a portion of the profile of the treatment chamber based upon the measured fluence levels.

94. The system of claim 93 further comprising:
a detector array structure positioned on a transmission side of the treatment chamber;
the first optical detector and the second optical detector positioned on the detector array structure within the portion of the profile of the treatment chamber.

95. The system of claim 94 further comprising a plurality of additional optical detectors postioned on the detector array structure, each additional collector positionally offset from each other and the first optical detector and the second optical detector to substantially cover at least the portion of the profile of the treatment chamber.

96. The system of claim 93 wherein the treatment chamber comprises a treatment chamber of a fluid flow path, wherein the product is flowed through the treatment chamber.

97. A light treatment monitoring system comprising:
a treatment chamber for containing a product to be treated with a light treatment, a portion of the treatment chamber and the product transmissive to at least 1% of light having at least one wavelength within a range of 170 to 2600 nm;
an optical detector positioned to measure a fluence level of light transmitting through a first portion of the treatment chamber; and
a position adjustment structure coupled to the optical detector, the position adjustment structure moveable in one or more directions to reposition the optical detector at different locations within a portion of a profile of treatment chamber.

98. The system of claim 97 further comprising a light source for providing the light treatment.

99. The system of claim 98 wherein the light source comprises a pulsed light source.

100. The system of claim 97 further comprising a controller coupled to the position adjustment structure for controlling the position of the optical detector relative to the treatment chamber.

101. The system of claim 100 wherein the position adjustment structure comprises an x-y translation table that moves the optical detector in an x direction and in a y direction to reposition the optical detector.

102. A method of fluid decontamination comprising:
flowing a fluid product through a treatment chamber, the fluid product and the treatment chamber transmissive to at least 1% of light having at least one wavelength within a range of 170 to 2600 nm;
illuminating the fluid product and the treatment chamber with at least one pulse of light;
measuring an amount of the light illuminating the fluid product and the treatment chamber; and
measuring an amount of the light transmitting through the fluid product and the treatment chamber.

103. A monitoring system for a fluid treatment system comprising:
a light source for providing pulses of light;
a treatment chamber positioned to receive the pulses of light, wherein a fluid product to be treated flows therethrough, wherein at least a portion of the treatment chamber and the fluid product are transmissive to at least 1% of light having at least one wavelength within a range of 170 to 2600 nm;
a first process monitor for measuring a fluence level of the pulses of light provided by the light source that illuminate the treatment chamber and the fluid product; and
a second process monitor for measuring a fluence level of portions of the pulses of light transmitting through the treatment chamber and through the fluid product.

104. A method of calibrating a spectroradiometer comprising:
calibrating a first spectrum of wavelengths of an operating spectrum of the spectroradiometer with a first calibration light source, the first calibration light source not providing an accurate calibration of the spectroradiometer in the first spectrum of wavelengths;
calibrating a second spectrum of wavelengths of the operating spectrum of the spectroradiometer with a second calibration light source, the second calibration light source providing an accurate calibration of the spectroradiometer in the second spectrum of wavelengths, a portion of the first spectrum of wavelengths overlapping the second spectrum of wavelengths; and
adjusting the calibration of the first spectrum of wavelengths based on a difference between the first calibration and the second calibration at the portion of first spectrum of wavelengths overlapping the second spectrum of wavelengths to generate an absolute irradiance calibration file that is sufficient to calibrate the spectroradiometer across the first spectrum of wavelength and the second spectrum of wavelengths.

105. The method of claim 104 wherein the calibrating the first spectrum of wavelengths with the first calibration light source comprises:
positioning an optical collector at a distance relative to the first calibration light source, the distance close enough to the first calibration light source such that the first calibration light source provides enough signal to calibrate the spectroradiometer coupled to the optical collector for the first spectrum of wavelengths, the first calibration light source positioned closer to the optical collector than specified in a first calibration file for the first spectrum of wavelengths, the optical collector positioned in a near field of the first calibration light source;
adjusting the first calibration file based upon the distance of the optical collector to the first calibration light source; and
calibrating the spectroradiometer using the adjusted calibration file to generate a system calibration file for the first spectrum of wavelengths.

106. The method of claim 105 wherein the calibrating the second spectrum of wavelengths with the second calibration light source comprises:
positioning the optical collector at a distance relative to the second calibration light source as specified in a second calibration file corresponding to the second calibration light source, such that the distance is sufficient to calibrate the spectroradiometer for the second spectrum of wavelengths; and
calibrating the spectroradiometer using the second calibration file to update the system calibration file for the second spectrum of wavelengths, the absolute values of the overlapping portion of the first spectrum of wavelengths and the second spectrum of wavelengths from the calibrating steps not matching.

107. The method of claim 106 wherein the adjusting the calibration of the first spectrum of wavelengths comprises:
determining a difference in absolute values in the system calibration file corresponding to the portion of the first spectrum of wavelengths and the second spectrum of wavelengths that overlap; and
adjusting the system calibration file for the first spectrum of wavelengths by the difference to generate the absolute irradiance calibration file.

108. The method of claim 107 further comprising:
verifying the absolute irradiance calibration file by recalibrating the spectroradiometer using the absolute irradiance calibration file and the second calibration light source.

109. The method of claim 104 wherein the first calibration light source does not provide an accurate absolute irradiance calibration of the spectroradiometer in the first spectrum of wavelengths.

110. A method for use with a spectrometer in a treatment system using light comprising:
generating a transmission file corresponding to a filter used to attenuate light input to the spectrometer, the filter non-uniformly transmitting light within a transmission spectrum through the filter, the transmission file generated on a per wavelength basis; and
compensating the calibration of the spectrometer based on the transmission file, such that readings of the spectrometer account for non-uniform transmission of the filter on a per wavelength basis.

111. The method of claim 110 wherein the generating comprises:
taking a reference reading across the transmission spectrum using a calibration light source without the filter in the light path;
taking a transmission reading across transmission spectrum using the calibration light source with the filter in the light path;
generating the transmission file based on a per wavelength comparison of the reference reading and the transmission reading.

112. The method of claim 111 further comprising:
adjusting the reference reading and the transmission reading by a respective baseline dark current reading of the spectrometer with no input light.

113. The method of claim 111 wherein the filter is positioned in the light path at a marked orientation.

114. The method of claim 113 further comprising:
determining the marked orientation by:
positioning the filter in the light path at an initial orientation;
taking spectrometer readings using the calibration light source;
incrementally rotating the filter to another location angularly offset from a previous orientation;
repeating the taking the spectrometer readings and incrementally rotating steps;
comparing all of the spectrometer readings to determine an optimal orientation of the filter in which the spectrometer readings vary the least in comparison to spectrometer readings at orientations angularly offset from the optimal orientation by a specified angle.

115. The method of claim 110 wherein the compensating comprises:
adjusting a system calibration file based on the transmission file on a per wavelength basis.

116. The method of claim 110 wherein the compensating comprises:
adjusting spectrometer readings in use with a treatment light source based on the transmission file on a per wavelength basis.
